(19) United States
(12) Patent Application Publication
(10) Pub. No.: US 2012/0149718 A1
(43) Pub. Date: Jun. 14, 2012

(54) Amido Compounds

(76) Inventors: Christine E. Brotherton-Pleiss, Sunnyvale, CA (US); Keith A. M. Walker, Los Altos Hills, CA (US)

(21) Appl. No.: 13/324,250

(22) Filed: Dec. 13, 2011

Related U.S. Application Data
(60) Provisional application No. 61/422,930, filed on Dec. 14, 2010.

(51) Int. Cl. A61K 31/4965; C07D 263/58; C07D 241/28; C07D 498/04; C07D 413/12; C07D 263/48; A61K 31/423; A61K 31/437; A61K 31/421; A61K 31/4439; A61P 19/02; A61P 29/00; A61P 25/06; A61P 11/00; A61P 11/06; A61P 3/10; C07D 241/20

(52) U.S. Cl. 514/255.06; 544/409; 548/222; 544/408; 544/407; 544/336; 546/115; 548/233; 514/375; 514/302; 514/377; 514/338; 546/271.7

(57) ABSTRACT

Compounds of the formula I:

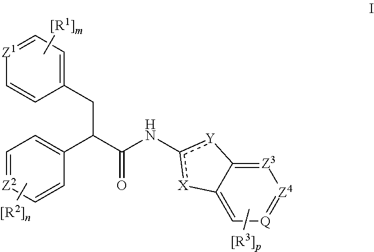

or pharmaceutically acceptable salts thereof, wherein the variables are as defined herein. Also disclosed are methods of making the compounds and using the compounds for treatment of diseases associated with the P2X7 purinergic receptor.

### PRIORITY TO RELATED APPLICATIONS

This application is entitled to the benefit of U.S. provisional patent application Ser. No. 61/422,930 filed on Dec. 14, 2010, the disclosure of which is incorporated herein by reference.

### FIELD OF THE INVENTION

P2X purinergic receptors are ATP-activated ionotropic receptors having seven subtypes. The P2X7 receptor subtype (also known as the P2Z receptor) is a ligand-gated ion channel found on mast cells, peripheral macrophages, lymphocytes, erythrocytes, fibroblasts and epidermal langerhans cells. Activation of P2X7 receptor on such immune system cells results in release of interleukin-1beta. (Solle et al., J. Biol. Chemistry 276, 125-132, (2001)). The P2X7 receptor is also found on microglia, Schwann cells and astrocytes within the central nervous system (Donnelly-Roberts et al., Br. J. Pharmacol. 151, 571-579 (2007)).

Antagonists of P2X7 have been shown to block P2X7-mediated IL-1beta release and P2X7-mediated cation flux (Stokes et al., Br. J. Pharmacol. 149, 880-887 (2006)). Mice lacking the P2X7 receptor show a lack of inflammatory and neuropathic hypersensitivity to mechanical and thermal stimuli (Chessell et al., Pain 114, 386-396 (2005)). P2X7 is thus believed to have a role in inflammatory responses (Ferrari et al., J. Immunol. 176, 3877-3883 (2006)) and in the onset and persistence of chronic pain (Honore et al., J. Pharmacol. Ex. Ther. 319, 1376-1385 (2006b)).

Modulators of the P2X7 receptor thus may have utility in the treatment of disease states such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, diabetes and Crohn's disease. P2X7 modulators may also be useful for treatment of pain, including chronic pain, neuropathic pain, and pain associated inflammatory processes and degenerative conditions.

There is accordingly a need for compounds that act as modulators of P2X receptors, including antagonists of P2X7 receptor, as well as a need for methods of treating diseases, conditions and disorders mediated by P2X7 The present invention satisfies these needs as well as others.

### SUMMARY OF THE INVENTION

The application provides a compound of formula I:

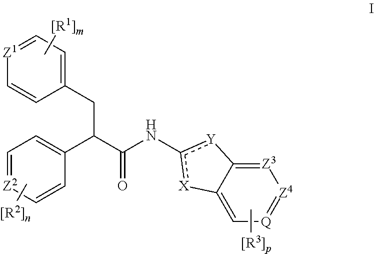

wherein:

### Z¹ is CH, CR¹, or N;

### Z² is CH, CR², or N;

### Z³ is CH, CR³, or N;

### Z⁴ is CH, CR³, or N;

### Q is CH, CR³, or N;

wherein only one of Z³, Z⁴, and Q can be N;

### X is N or O;

### Y is N or O;

wherein both X and Y are not both N or O;

each — is a single or double bond, wherein both are not single or double;
each R¹ is independently halo, hydroxy, lower alkyl, lower alkoxy, lower hydroxyalkyl, —SO₂R, or lower haloalkyl
m is 0, 1, 2, or 3;
each R² is independently halo, lower alkyl, lower hydroxyalkyl, lower alkoxy, C(═O)OR^{2′}, C(═O)N(R^{2′})₂, or S(═O)₂R^{2′};

R^{2′} is H, lower alkyl, lower alkoxy, amino, or lower hydroxyalkyl;

n is 0, 1, 2, or 3;
each R³ is halo, hydroxy, lower alkyl, lower alkoxy, lower hydroxyalkyl, lower haloalkyl, CO₂R³′, or S(═O)₂R^{3′};

each R^{3′} is independently H, lower alkyl, cycloalkyl, alkylamine, or heterocycloalkyl;

p is 0, 1, 2, or 3;
or a pharmaceutically acceptable salt thereof.

The application provides a compound of Formula II

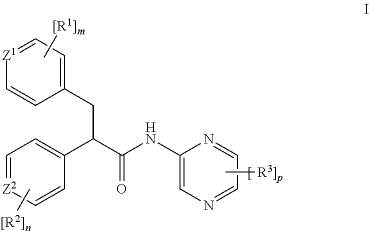

wherein

### Z¹ is CH, CR¹, or N;

### Z² is CH, CR², or N;

each R¹ is independently halo, hydroxy, lower alkyl, lower alkoxy, lower hydroxyalkyl, or lower haloalkyl;
m is 0, 1, 2, or 3;
each R² is independently halo, lower alkyl, C(═O)R^{2′}, C(═)ON(R^{2′})₂, or S(═O)₂R^{2′};

each R^{2′} is independently H, lower alkyl, lower alkoxy, amino, or lower hydroxyalkyl;

n is 0, 1, 2, or 3;
each R³ is independently halo, hydroxy, lower alkyl, lower alkoxy, lower hydroxyalkyl, alkoxyalkyl, lower haloalkyl, alkylamino, thioalkyl, CH₂(O)C(═O)R^{3′}, C(═O)OR^{3′}, CH₂(O)R^{3′}, or CH₂N(R^{3′})₂;

each R^{3′} is independently H, lower alkyl, lower alkoxy, or phenyl lower alkyl,

p is 0, 1, 2, or 3;
or a pharmaceutically acceptable salt thereof

The application provides a pharmaceutical composition comprising:
(a) a pharmaceutically acceptable carrier; and
(b) a compound of Formula I or II.

The application provides a method for treating arthritis, said method comprising administering to a subject in need thereof a therapeutically effective amount of a compound of Formula I or II.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of Formula I or II.

The application provides a method for treating a respiratory disorder selected from chronic obstructive pulmonary disorder (COPD), asthma, and bronchospasm, said method comprising administering to a subject in need thereof a therapeutically effective amount of a compound of Formula I or II.

The application provides a method for treating diabetes, said method comprising administering to a subject in need thereof a therapeutically effective amount of a compound of Formula I or II.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means a linear monovalent hydrocarbon radical of two to six carbon atoms or a branched monovalent hydrocarbon radical of three to six carbon atoms, containing at least one triple bond, e.g., ethynyl, propynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkoxyalkoxy” means a group of the formula —O—R—R′ wherein R is alkylene and R′ is alkoxy as defined herein.

“Alkylcarbonyl” means a moiety of the formula —C(O)—R, wherein R is alkyl as defined herein.

“Alkoxycarbonyl” means a group of the formula —C(O)—R wherein R is alkoxy as defined herein.

“Alkylcarbonylalkyl” means a group of the formula —R—C(O)—R wherein R is alkylene and R′ is alkyl as defined herein.

“Alkoxycarbonylalkyl” means a group of the formula —R—C(O)—R wherein R is alkylene and R′ is alkoxy as defined herein.

“Alkoxycarbonylalkoxy” means a group of the formula —O—R—C(O)—R′ wherein R is alkylene and R′ is alkoxy as defined herein.

“Hydroxycarbonylalkoxy” means a group of the formula —O—R—C(O)—OH wherein R is alkylene as defined herein.

“Alkylaminocarbonylalkoxy” means a group of the formula —O—R—C(O)—NHR′ wherein R is alkylene and R′ is alkyl as defined herein.

“Dialkylaminocarbonylalkoxy” means a group of the formula —O—R—C(O)—NR′R″ wherein R is alkylene and R′ and R″ are alkyl as defined herein.

“Alkylaminoalkoxy” means a group of the formula —O—R—NHR′ wherein R is alkylene and R′ is alkyl as defined herein.

“Dialkylaminoalkoxy” means a group of the formula —O—R—NR′R′ wherein R is alkylene and R′ and R″ are alkyl as defined herein.

“Alkylsulfonyl” means a moiety of the formula —SO₂—R, wherein R is alkyl as defined herein.

“Alkylsulfonylalkyl means a moiety of the formula —R′—SO₂—R″ where R′ is alkylene and R″ is alkyl as defined herein.

“Alkylsulfonylalkoxy” means a group of the formula —O—R—SO₂—R′ wherein R is alkylene and R′ is alkyl as defined herein.

“Amino means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Aminocarbonyl” means a group of the formula —C(O)—R wherein R is amino as defined herein.

“Alkoxyamino” means a moiety of the formula —NR—OR′ wherein R is hydrogen or alkyl and R′ is alkyl as defined herein.

“Alkylsulfanyl” means a moiety of the formula —SR wherein R is alkyl as defined herein.

“Aminoalkyl” means a group —R—R′ wherein R′ is amino and R is alkylene as defined herein. “Aminoalkyl” includes aminomethyl, aminoethyl, 1-aminopropyl, 2-aminopropyl, and the like. The amino moiety of “aminoalkyl” may be substituted once or twice with alkyl to provide “alkylaminoalkyl” and “dialkylaminoalkyl” respectively. “Alkylaminoalkyl” includes methylaminomethyl, methylaminoethyl, methylaminopropyl, ethylaminoethyl and the like. “Dialkylaminoalkyl” includes dimethylaminomethyl, dimethylaminoethyl, dimethylaminopropyl, N-methyl-N-ethylaminoethyl, and the like.

“Aminoalkoxy” means a group —OR—R′ wherein R′ is amino and R is alkylene as defined herein.

“Alkylsulfonylamido” means a moiety of the formula —NR′SO₂—R wherein R is alkyl and R′ is hydrogen or alkyl.

“Aminocarbonyloxyalkyl” or “carbamylalkyl” means a group of the formula —R—O—C(O)—NR′R″ wherein R is alkylene and R′, R″ each independently is hydrogen or alkyl as defined herein.

“Alkynylalkoxy” means a group of the formula —O—R—R′ wherein R is alkylene and R′ is alkynyl as defined herein.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Arylsulfonyl means a group of the formula —SO₂—R wherein R is aryl as defined herein.

“Aryloxy” means a group of the formula —O—R wherein R is aryl as defined herein.

“Aralkyloxy” means a group of the formula —O—R—R″ wherein R is alkylene and R′ is aryl as defined herein.

“Carboxy” or “hydroxycarbonyl”, which may be used interchangeably, means a group of the formula —C(O)—OH.

“Cyanoalkyl” means a moiety of the formula —R′—R″, where R′ is alkylene as defined herein and R″ is cyano or nitrile.

“Cycloalkyl” means a monovalent saturated carbocyclic moiety consisting of mono- or bicyclic rings. Preferred cycloalkyl are unsubstituted or substituted with alkyl. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

“Cycloalkylalkyl” means a moiety of the formula —R′—R″, where R′ is alkylene and R″ is cycloalkyl as defined herein.

“Cycloalkylalkoxy” means a group of the formula —O—R—R′ wherein R is alkylene and R′ is cycloalkyl as defined herein.

“Heteroalkyl” means an alkyl radical as defined herein wherein one, two or three hydrogen atoms have been replaced with a substituent independently selected from the group consisting of —OR^{a}, —NR^{b}R^{c} and —S(O)—R^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heteroaryl” means a monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

Heteroarylalkyl” or “heteroaralkyl” means a group of the formula —R—R′ wherein R is alkylene and R′ is heteroaryl as defined herein.

“Heteroarylsulfonyl means a group of the formula —SO₂—R wherein R is heteroaryl as defined herein.

“Heteroaryloxy” means a group of the formula —O—R wherein R is heteroaryl as defined herein.

“Heteroaralkyloxy” means a group of the formula —O—R—R″ wherein R is alkylene and R′ is heteroaryl as defined herein.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. An exemplary haloalkoxy is difluoromethoxy.

“Heterocycloamino” means a saturated ring wherein at least one ring atom is N, NH or N-alkyl and the remaining ring atoms form an alkylene group.

“Heterocyclyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

“Heterocyclylalkyl” means a moiety of the formula —R—R′ wherein R is alkylene and R′ is heterocyclyl as defined herein.

“Heterocyclyloxy” means a moiety of the formula —OR wherein R is heterocyclyl as defined herein.

“Heterocyclylalkoxy” means a moiety of the formula —OR—R′ wherein R is alkylene and R′ is heterocyclyl as defined herein.

“Hydroxyalkoxy” means a moiety of the formula —OR wherein R is hydroxyalkyl as defined herein. Exemplary hydroxyalkoxy include, for example, 3-methoxy-2-hydroxy-propyl and 3-hydroxy-2-methoxy-propyl.

“Hydroxyalkoxyalkyl” means a moiety of the formula —ROR′ wherein R is alkylene and R′ is hydroxyalkyl as defined herein, and R or R′ or both are substituted with hydroxy.

“Hydroxyalkylamino” means a moiety of the formula —NR—R′ wherein R is hydrogen or alkyl and R′ is hydroxyalkyl as defined herein.

“Hydroxyalkylaminoalkyl” means a moiety of the formula —R—NR′—R″ wherein R is alkylene, R′ is hydrogen or alkyl, and R″ is hydroxyalkyl as defined herein.

“Hydroxycarbonylalkyl” or “carboxyalkyl” means a group of the formula —R—(CO)—OH where R is alkylene as defined herein.

“Hydroxycarbonylalkoxy” means a group of the formula —O—R—C(O)—OH wherein R is alkylene as defined herein.

“Hydroxyalkyloxycarbonylalkyl” or “hydroxyalkoxycarbonylalkyl” means a group of the formula —R—C(O)—O—R—OH wherein each R is alkylene and may be the same or different.

“Hydroxyalkyl” means an alkyl moiety as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl

“Hydroxycycloalkyl” means a cycloalkyl moiety as defined herein wherein one, two or three hydrogen atoms in the cycloalkyl radical have been replaced with a hydroxy substituent. Representative examples include, but are not limited to, 2-, 3-, or 4-hydroxycyclohexyl, and the like.

“Alkoxy hydroxyalkyl” and “hydroxy alkoxyalkyl”, which may be used interchangeably, means an alkyl as defined herein that is substituted at least once with hydroxy and at least once with alkoxy. “Alkoxy hydroxyalkyl” and “hydroxy alkoxyalkyl” thus encompass, for example, 2-hydroxy-3-methoxy-propan-1-yl and the like.

“Urea” or “ureido” means a group of the formula —NR′—C(O)—NR″R′″ wherein R′, R″ and R′″ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Sulfonamido” means a group of the formula —SO₂—NR′R″ wherein R′, R″ and R′″ each independently is hydrogen or alkyl.

“Optionally substituted”, when used in association with “aryl”, phenyl”, “heteroaryl” “cycloalkyl” or “heterocyclyl”, means an aryl, phenyl, heteroaryl, cycloalkyl or heterocyclyl which is optionally substituted independently with one to four substituents, preferably one or two substituents selected from alkyl, cycloalkyl, cycloalkylalkyl, heteroalkyl, hydroxyalkyl, halo, nitro, cyano, hydroxy, alkoxy, amino, acylamino, mono-alkylamino, di-alkylamino, haloalkyl, haloalkoxy, heteroalkyl, —COR, —SO₂R (where R is hydrogen, alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{a}R^{b} (where n is an integer from 0 to 5, R′ and R″ are independently hydrogen or alkyl, and R^{a} and R^{b} are, independently of each other, hydrogen, alkyl, cycloalkyl, cycloalkylalkyl, phenyl or phenylalkyl). Certain preferred optional substituents for “aryl”, phenyl”, “heteroaryl” “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or

salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. The artisan in the art will know how to chose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:
(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure.

All patents and publications identified herein are incorporated herein by reference in their entirety.

### P2X7 Inhibitors

The application provides a compound of formula I:

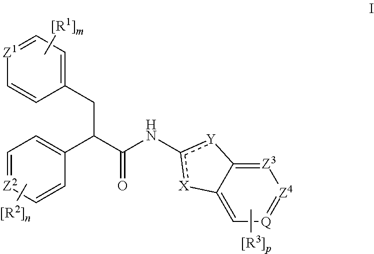

wherein:

### Z¹ is CH, CR¹, or N;

### Z² is CH, CR², or N;

### Z³ is CH, CR³, or N;

### Z⁴ is CH, CR³, or N;

### Q is CH, CR³, or N;

wherein only one of Z³, Z⁴, and Q can be N;

### X is N or O;

### Y is N or O;

wherein both X and Y are not both N or O;

each — is a single or double bond, wherein both are not single or double;
each R¹ is independently halo, hydroxy, lower alkyl, lower alkoxy, lower hydroxyalkyl, —SO₂R, or lower haloalkyl;
m is 0, 1, 2, or 3;
each R² is independently halo, lower alkyl, lower hydroxyalkyl, lower alkoxy, C(═O)OR^{2′}, C(═O)N(R^{2′})₂, or S(═O)₂R^{2′};

R^{2′} is H, lower alkyl, lower alkoxy, amino, or lower hydroxyalkyl;

n is 0, 1, 2, or 3;
each R³ is halo, hydroxy, lower alkyl, lower alkoxy, lower hydroxyalkyl, lower haloalkyl, CO₂R³′, or S(═O)₂R^{3′};

each R^{3′} is independently H, lower alkyl, cycloalkyl, alkylamine, or heterocycloalkyl;

p is 0, 1, 2, or 3;
or a pharmaceutically acceptable salt thereof

The application provides a compound of formula I, wherein X is O and Y is N.

The application provides a compound of formula I, wherein X is N and Y is O.

The application provides a compound of formula I, wherein Z³, Z⁴, and Q are CH or CR³.

The application provides a compound of formula I, wherein X is O, Y is N, Z³, Z⁴, and Q are CH or CR³.

The application provides a compound of formula I, wherein X is N, Y is O, Z³, Z⁴, and Q are CH or CR³.

The application provides a compound of formula I, wherein m is 1.

The application provides a compound of formula I, wherein X is O, Y is N, and m is 1.

The application provides a compound of formula I, wherein X is N, Y is O, and m is 1.

The application provides a compound of formula I, wherein m is 1, Z³, Z⁴, and Q are CH or CR³.

The application provides a compound of formula I, wherein m is 1, X is O, Y is N, Z³, Z⁴, and Q are CH or CR³.

The application provides a compound of formula I, wherein m is 1, X is N, Y is O, Z³, Z⁴, and Q are CH or CR³.

The application provides a compound of formula I, wherein R¹ is halo or lower alkyl.

The application provides a compound of formula I, wherein m is 1, X is O, Y is N, Z³, Z⁴, and Q are CH or CR³, and R¹ is halo or lower alkyl.

The application provides a compound of formula I, wherein m is 1, X is N, Y is O, Z³, Z⁴, and Q are CH or CR³, and R¹ is halo or lower alkyl.

The application provides a compound of formula I, wherein R² is S(═O)₂R^{2′} and R^{2′} is lower alkyl.

The application provides a compound of formula I, wherein m is 1, X is O, Y is N, Z³, Z⁴, and Q are CH or CR³, R¹ is halo or lower alkyl. R² is S(═O)₂R^{2′} and R^{2′} is lower alkyl.

The application provides a compound of formula I, wherein m is 1, X is N, Y is O, Z³, Z⁴, and Q are CH or CR³, R¹ is halo or lower alkyl, R² is S(═O)₂R^{2′} and R^{2′} is lower alkyl.

The application provides a compound of formula I, wherein p is 0.

The application provides a compound of formula I, wherein p is 0, m is 1, X is O, Y is N, Z³, Z⁴, and Q are CH or CR³, R¹ is halo or lower alkyl. R² is S(═O)₂R^{2′} and R^{2′} is lower alkyl.

The application provides a compound of formula I, wherein p is 0, m is 1, X is N, Y is O, Z³, Z⁴, and Q are CH or CR³, R¹ is halo or lower alkyl, R² is S(═O)₂R^{2′} and R^{2′} is lower alkyl.

The application provides a compound of formula I, wherein p is 1.

The application provides a compound of formula I, wherein p is 1, m is 1, X is O, Y is N, Z³, Z⁴, and Q are CH or CR³, R¹ is halo or lower alkyl. R² is S(═O)₂R^{2′} and R^{2′} is lower alkyl.

The application provides a compound of formula I, wherein p is 1, m is 1, X is N, Y is O, Z³, Z⁴, and Q are CH or CR³, R¹ is halo or lower alkyl, R² is S(═O)₂R^{2′} and R^{2′} is lower alkyl.

The application provides a compound of formula I, wherein Z³ is N.

The application provides a compound of formula I, wherein Z⁴ is N.

The application provides a compound of formula I, wherein Q is N.

The application provides a compound of formula I, wherein R² is C(═O)N(R^{2′})₂.

The application provides a compound of formula I, wherein R² is C(═O)OR^{2′}.

The application provides a compound of Formula II

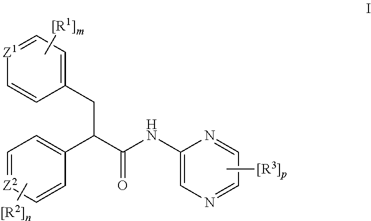

wherein:

### Z¹ is CH, CR¹, or N;

### Z² is CH, CR², or N;

each R¹ is independently halo, hydroxy, lower alkyl, lower alkoxy, lower hydroxyalkyl, or lower haloalkyl;
m is 0, 1, 2, or 3;
each R² is independently halo, lower alkyl, C(═O)R^{2′}, C(═)ON(R^{2′})₂, or S(═O)₂R^{2′};

each R^{2′} is independently H, lower alkyl, lower alkoxy, amino, or lower hydroxyalkyl;

n is 0, 1, 2, or 3;
each R³ is independently halo, hydroxy, lower alkyl, lower alkoxy, alkoxyalkyl, alkylamino, thioalkyl, lower hydroxyalkyl, lower haloalkyl, CH₂(O)C(═O)R^{3′}, C(═O)OR^{3′}, CH₂(O)R^{3′}, or CH₂N(R^{3′})₂;

each R^{3′} is independently H, lower alkyl, lower alkoxy, or phenyl lower alkyl,

p is 0, 1, 2, or 3;
or a pharmaceutically acceptable salt thereof.

The application provides a compound of formula II, wherein m is 1 and n is 1.

The application provides a compound of formula II, wherein R² is S(═O)₂CH₃.

The application provides a compound of formula II, wherein p is 1.

The application provides a compound of formula II, wherein R² is S(═O)₂CH₃ and p is 1.

The application provides a compound of formula II, R³ is halo.

The application provides a compound of formula II, R³ is halo, R² is S(═O)₂CH₃ and p is 1

The application provides a compound of formula II, wherein R³ is lower hydroxyalkyl.

The application provides a compound of formula II, wherein R³ is lower hydroxyalkyl, R² is S(═O)₂CH₃ and p is 1.

The application provides a compound of formula II, wherein R³ is lower alkyl.

The application provides a compound of formula II, wherein R³ is lower alkyl, R² is S(═O)₂CH₃ and p is 1.

The application provides a compound selected from the group consisting of:
N-(5-Bromo-pyrazin-2-yl)-2-(4-methanesulfonyl-phenyl)-3-o-tolyl-propionamide;
N-Benzooxazol-2-yl-2-(4-methanesulfonyl-phenyl)-3-o-tolyl-propionamide;
N-(5-Chloro-benzooxazol-2-yl)-2-(4-methanesulfonyl-phenyl)-3-o-tolyl-propionamide;
N-Benzooxazol-2-yl-2-(4-methanesulfonyl-phenyl)-3-phenyl-propionamide;
N-Benzooxazol-2-yl-3-(2-chloro-phenyl)-2-(4-methanesulfonyl-phenyl)-propionamide;
N-Benzooxazol-2-yl-3-(2-chloro-4-fluoro-phenyl)-2-(4-methanesulfonyl-phenyl)-propionamide;
N-Benzooxazol-2-yl-3-(4-fluoro-phenyl)-2-(4-methanesulfonyl-phenyl)-propionamide;
N-Benzooxazol-2-yl-3-(2-fluoro-phenyl)-2-(4-methanesulfonyl-phenyl)-propionamide;
N-Benzooxazol-2-yl-2-(4-methanesulfonyl-phenyl)-3-(4-methoxy-phenyl)-propionamide;

(S)—N-Benzooxazol-2-yl-2-(4-methanesulfonyl-phenyl)-3-o-tolyl-propionamide;
(R)—N-Benzooxazol-2-yl-2-(4-methanesulfonyl-phenyl)-3-o-tolyl-propionamide;
N-Benzooxazol-2-yl-3-(4-fluoro-phenyl)-2-(3-methanesulfonyl-phenyl)-propionamide;
N-(5-Bromo-pyrazin-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-methylsulfanyl-pyrazin-2-yl)-propionamide;
5-[3-(4-Fluoro-phenyl)-2-(4-methanesulfonyl-phenyl)-propionylamino]-pyrazine-2-carboxylic acid ethyl ester;
3-(4-Fluoro-phenyl)-N-(5-hydroxymethyl-pyrazin-2-yl)-2-(4-methanesulfonyl-phenyl)-propionamide;
3-(4-Fluoro-phenyl)-N-[5-(1-hydroxy-1-methyl-ethyl)-pyrazin-2-yl]-2-(4-methanesulfonyl-phenyl)-propionamide;
3-(4-Fluoro-phenyl)-2-(4-methanesulfonyl-phenyl)-N-oxazolo[4,5-b]pyridin-2-yl-propionamide;
3-(4-Fluoro-phenyl)-2-(4-methanesulfonyl-phenyl)-N-(5-methyl-pyrazin-2-yl)-propionamide;
3-(4-Fluoro-phenyl)-N-(6-hydroxymethyl-benzooxazol-2-yl)-2-(4-methanesulfonyl-phenyl)-propionamide;
3-(4-Fluoro-phenyl)-2-(4-methanesulfonyl-phenyl)-N-(5-methoxymethyl-pyrazin-2-yl)-propionamide;
N-(5-Ethoxymethyl-pyrazin-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-{[(2-methoxy-ethyl)-methyl-amino]-methyl}-pyrazin-2-yl)-propionamide;
N-{5-[(Benzyl-methyl-amino)-methyl]-pyrazin-2-yl}-3-(4-fluoro-phenyl)-2-(4-methanesulfonyl-phenyl)-propionamide;
N-(6-Chloro-pyrazin-2-yl)-3-(4-fluoro-phenyl)-2-(4-methanesulfonyl-phenyl)-propionamide;
3-(3-Fluoro-phenyl)-N-(5-hydroxymethyl-pyrazin-2-yl)-2-(4-methanesulfonyl-phenyl)-propionamide;
N-Benzooxazol-2-yl-3-(4-fluoro-phenyl)-2-(4-methanesulfonyl-2-methyl-phenyl)-propionamide;
N-Benzooxazol-2-yl-2,3-bis-(4-fluoro-phenyl)-propionamide;
N-Benzooxazol-2-yl-2-(2,4-difluoro-phenyl)-3-(4-fluoro-phenyl)-propionamide;
4-[1-(Benzooxazol-2-ylcarbamoyl)-2-(4-fluoro-phenyl)-ethyl]-benzoic acid ethyl ester;
N-Benzooxazol-2-yl-3-(4-fluoro-phenyl)-2-[4-(1-hydroxy-1-methyl-ethyl)-phenyl]-propionamide;
4-[1-(Benzooxazol-2-ylcarbamoyl)-2-(4-fluoro-phenyl)-ethyl]-benzamide;
4-[1-(Benzooxazol-2-ylcarbamoyl)-2-(4-fluoro-phenyl)-ethyl]-N-(2-hydroxy-ethyl)-benzamide;
4-[1-(Benzooxazol-2-ylcarbamoyl)-2-(4-fluoro-phenyl)-ethyl]-N-(2-dimethylamino-ethyl)-benzamide;
3-(4-Fluoro-phenyl)-2-(4-methanesulfonyl-phenyl)-N-(4-methyl-benzooxazol-2-yl)-propionamide;
3-(4-Fluoro-phenyl)-2-(4-methanesulfonyl-phenyl)-N-(6-methyl-benzooxazol-2-yl)-propionamide;
3-(4-Fluoro-phenyl)-2-(4-methanesulfonyl-phenyl)-N-(5-methyl-benzooxazol-2-yl)-propionamide;
N-(6-Chloro-benzooxazol-2-yl)-3-(4-fluoro-phenyl)-2-(4-methanesulfonyl-phenyl)-propionamide;
N-(5-Chloro-benzooxazol-2-yl)-3-(4-fluoro-phenyl)-2-(4-methanesulfonyl-phenyl)-propionamide;
N-(6-Fluoro-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-trifluoromethyl-benzooxazol-2-yl)-propionamide;
3-(4-Fluoro-phenyl)-2-(4-methanesulfonyl-phenyl)-N-(7-methyl-benzooxazol-2-yl)-propionamide;
N-(5,7-Dimethyl-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-methoxy-benzooxazol-2-yl)-propionamide;
2-[3-(4-Fluoro-phenyl)-2-(4-methanesulfonyl-phenyl)-propionylamino]-benzooxazole-6-carboxylic acid methyl ester;
N-(5-Fluoro-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-[5-(pyrrolidine-1-sulfonyl)-benzooxazol-2-yl]-propionamide;
3-(4-Fluoro-phenyl)-2-(4-methanesulfonyl-phenyl)-N-(5-phenyl-oxazol-2-yl)-propionamide;
Acetic acid 5-[3-(4-fluoro-phenyl)-2-(4-methanesulfonyl-phenyl)-propionylamino]-pyrazin-2-ylmethyl ester;
(R)-3-(4-Fluoro-phenyl)-N-(5-hydroxymethyl-pyrazin-2-yl)-2-(4-methanesulfonyl-phenyl)-propionamide;
(S)-3-(4-Fluoro-phenyl)-N-(5-hydroxymethyl-pyrazin-2-yl)-2-(4-methanesulfonyl-phenyl)-propionamide;
3-(4-Fluoro-phenyl)-2-(4-methanesulfonyl-phenyl)-N-oxazolo[4,5-c]pyridin-2-yl-propionamide;
3-(4-Fluoro-phenyl)-2-(4-methanesulfonyl-phenyl)-N-oxazolo[5,4-c]pyridin-2-yl-propionamide;
N-Benzooxazol-2-yl-2-(4-fluoro-phenyl)-3-(4-methanesulfonyl-phenyl)-propionamide;
N-Benzooxazol-2-yl-3-(4-fluoro-phenyl)-2-pyridin-4-yl-propionamide; and
N-Benzooxazol-2-yl-2-(4-methanesulfonyl-phenyl)-3-pyridin-4-yl-propionamide.

The application provides a pharmaceutical composition comprising:
(a) a pharmaceutically acceptable carrier; and
(b) a compound of Formula I or Formula II.

The application provides a method for treating arthritis, said method comprising administering to a subject in need thereof a therapeutically effective amount of a compound of Formula I or Formula II.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of Formula I or Formula II.

The application provides a method for treating a respiratory disorder selected from chronic obstructive pulmonary disorder (COPD), asthma, and bronchospasm, said method comprising administering to a subject in need thereof a therapeutically effective amount of a compound of Formula I or Formula II.

The application provides a method for treating diabetes, said method comprising administering to a subject in need thereof a therapeutically effective amount of a compound of Formula I or Formula II.

The application provides a use of a compound of Formula I or Formula II in the preparation of a medicament for the treatment of autoimmune and inflammatory diseases associated with P2X7 modulation.

A compound, method, or use as described herein.

### Compounds

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formula I or Formula II.

TABLE I

| Compound | Nomenclature | Structure |
|---|---|---|
| I-1 | N-(5-Bromo- pyrazin-2-yl)-2-(4- methanesulfonyl- phenyl)-3-o-tolyl- propionamide | 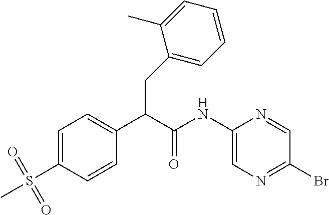 |
| I-2 | N-Benzooxazol-2- yl-2-(4- methanesulfonyl- phenyl)-3-o-tolyl- propionamide | 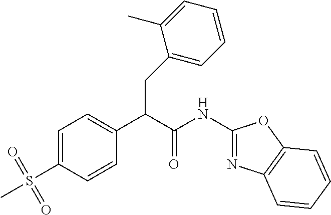 |
| I-3 | N-(5-Chloro- benzooxazol-2-yl)- 2-(4- methanesulfonyl- phenyl)-3-o-tolyl- propionamide | 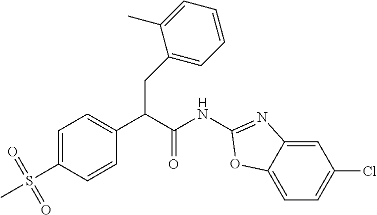 |
| I-4 | N-Benzooxazol-2- yl-2-(4- methanesulfonyl- phenyl)-3-phenyl- propionamide | 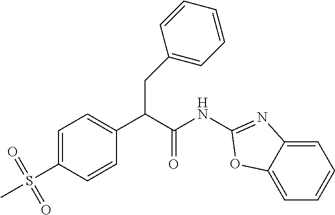 |
| I-5 | N-Benzooxazol-2- yl-3-(2-chloro- phenyl)-2-(4- methanesulfonyl- phenyl)- propionamide | 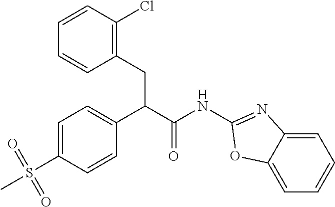 |
| I-6 | N-Benzooxazol-2- yl-3-(2-chloro-4- fluoro-phenyl)-2- (4- methanesulfonyl- phenyl)- propionamide | 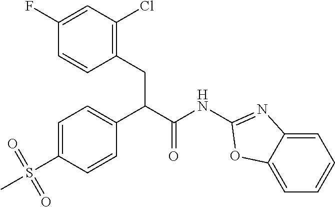 |
| I-7 | N-Benzooxazol-2- yl-3-(4-fluoro- phenyl)-2-(4- methanesulfonyl- phenyl)- propionamide | 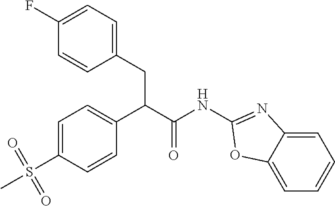 |
| I-8 | N-Benzooxazol-2- yl-3-(2-fluoro- phenyl)-2-(4- methanesulfonyl- phenyl)- propionamide | 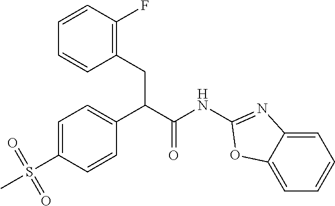 |
| I-9 | N-Benzooxazol-2- yl-2-(4- methanesulfonyl- phenyl)-3-(4- methoxy-phenyl)- propionamide | 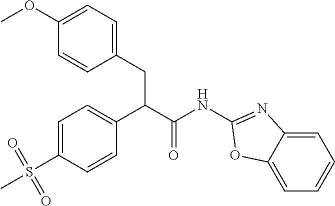 |
| I-10 | (S)-N- Benzooxazol-2-yl- 2-(4- methanesulfonyl- phenyl)-3-o-tolyl- propionamide | 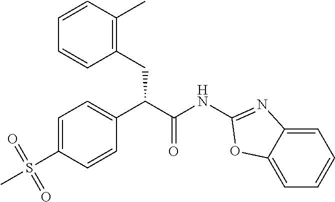 |
| I-11 | (R)-N- Benzooxazol-2-yl- 2-(4- methanesulfonyl- phenyl)-3-o-tolyl- propionamide | 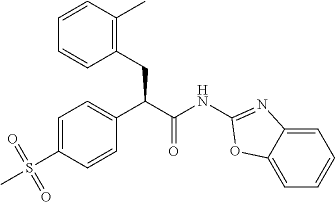 |
| I-12 | N-Benzooxazol-2- yl-3-(4-fluoro- phenyl)-2-(3- methanesulfonyl- phenyl)- propionamide | 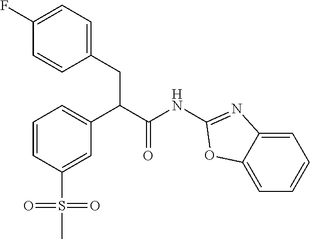 |
| I-13 | N-(5-Bromo- pyrazin-2-yl)-3-(4- fluoro-phenyl)-2- (4- methanesulfonyl- phenyl)- propionamide | 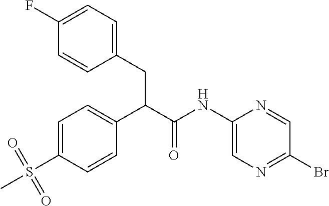 |
| I-14 | 3-(4-Fluoro- phenyl)-2-(4- methanesulfonyl- phenyl)-N-(5- methylsulfanyl- pyrazin-2-yl)- propionamide | 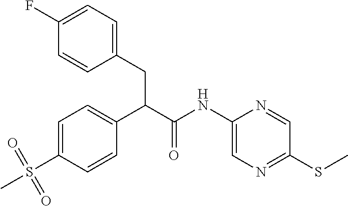 |
| I-15 | 5-[3-(4-Fluoro- phenyl)-2-(4- methanesulfonyl- phenyl)- propionylamino]- pyrazine-2- carboxylic acid ethyl ester | 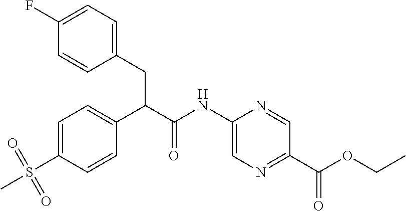 |
| I-16 | 3-(4-Fluoro- phenyl)-N-(5- hydroxymethyl- pyrazin-2-yl)-2-(4- methanesulfonyl- phenyl)- propionamide | 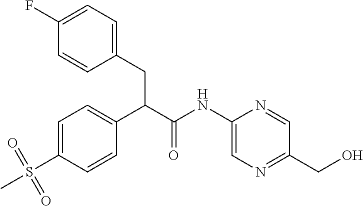 |
| I-17 | 3-(4-Fluoro- phenyl)-N-[5-(1- hydroxy-1-methyl- ethyl)-pyrazin-2- yl]-2-(4- methanesulfonyl- phenyl)- propionamide | 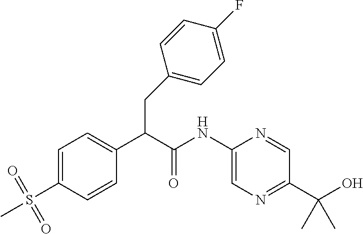 |
| I-18 | 3-(4-Fluoro- phenyl)-2-(4- methanesulfonyl- phenyl)-N- oxazolo[4,5- b]pyridin-2-yl- propionamide | 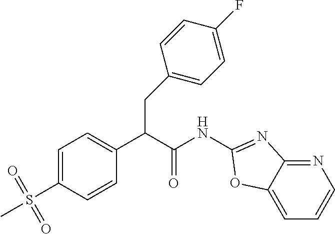 |
| I-19 | 3-(4-Fluoro- phenyl)-2-(4- methanesulfonyl- phenyl)-N-(5- methyl-pyrazin-2- yl)-propionamide | 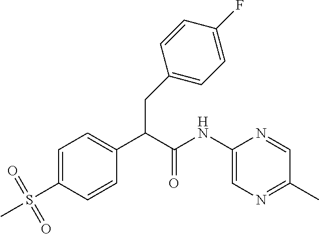 |
| I-20 | 3-(4-Fluoro- phenyl)-N-(6- hydroxymethyl- benzooxazol-2-yl)- 2-(4- methanesulfonyl- phenyl)- propionamide | 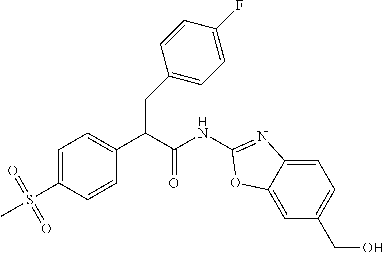 |
| I-21 | 3-(4-Fluoro- phenyl)-2-(4- methanesulfonyl- phenyl)-N-(5- methoxymethyl- pyrazin-2-yl)- propionamide | 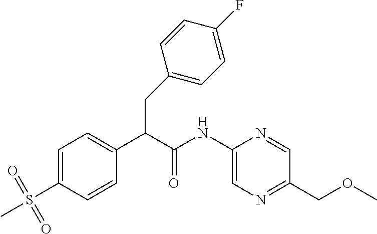 |
| I-22 | N-(5- Ethoxymethyl- pyrazin-2-yl)-3-(4- fluoro-phenyl)-2- (4- methanesulfonyl- phenyl)- propionamide | 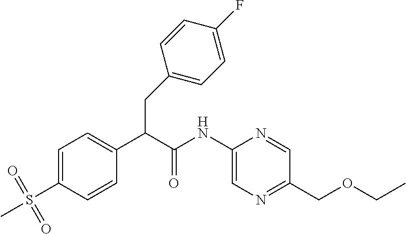 |
| I-23 | 3-(4-Fluoro- phenyl)-2-(4- methanesulfonyl- phenyl)-N-(5-{[(2- methoxy-ethyl)- methyl-amino]- methyl}-pyrazin-2- yl)-propionamide | 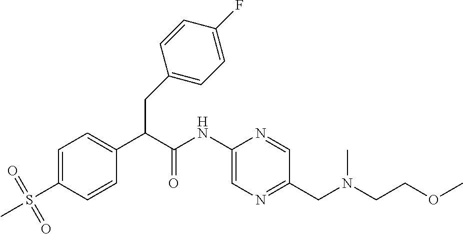 |
| I-24 | N-{5-[(Benzyl- methyl-amino)- methyl]-pyrazin-2- yl}-3-(4-fluoro- phenyl)-2-(4- methanesulfonyl- phenyl)- propionamide | 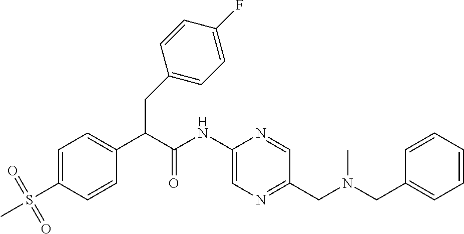 |
| I-25 | N-(6-Chloro- pyrazin-2-yl)-3-(4- fluoro-phenyl)-2- (4- methanesulfonyl- phenyl)- propionamide | 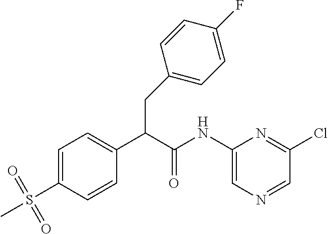 |
| I-26 | 3-(3-Fluoro- phenyl)-N-(5- hydroxymethyl- pyrazin-2-yl)-2-(4- methanesulfonyl- phenyl)- propionamide | 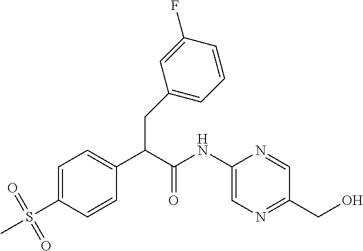 |
| I-27 | N-Benzooxazol-2- yl-3-(4-fluoro- phenyl)-2-(4- methanesulfonyl-2- methyl-phenyl)- propionamide | 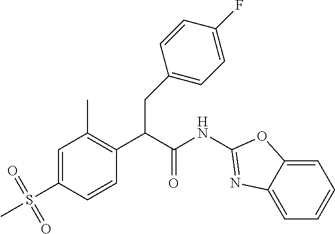 |
| I-28 | N-Benzooxazol-2- yl-2,3-bis-(4- fluoro-phenyl)- propionamide | 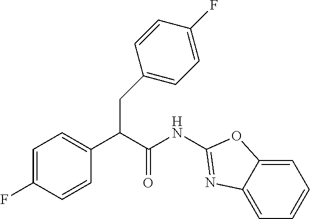 |
| I-29 | N-Benzooxazol-2- yl-2-(2,4-difluoro- phenyl)-3-(4- fluoro-phenyl)- propionamide | 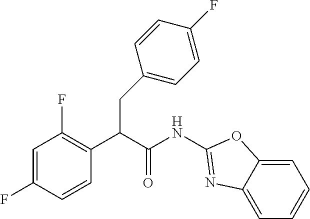 |
| I-30 | 4-[1- (Benzooxazol-2- ylcarbamoyl)-2-(4- fluoro-phenyl)- ethyl]-benzoic acid ethyl ester | 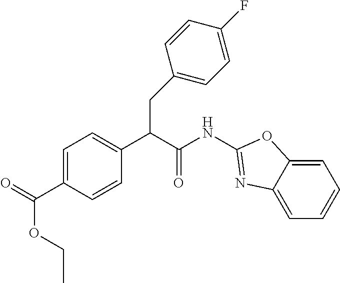 |
| I-31 | N-Benzooxazol-2- yl-3-(4-fluoro- phenyl)-2-[4-(1- hydroxy-1-methyl- ethyl)-phenyl]- propionamide | 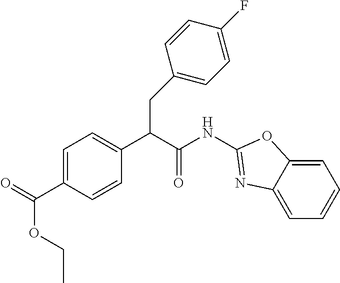 |
| I-32 | 4-[1- (Benzooxazol-2- ylcarbamoyl)-2-(4- fluoro-phenyl)- ethyl]-benzamide | 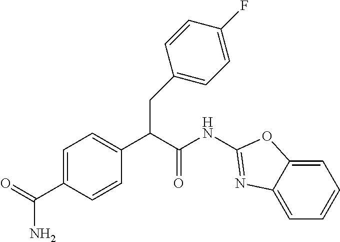 |
| I-33 | 4-[1- (Benzooxazol-2- ylcarbamoyl)-2-(4- fluoro-phenyl)- ethyl]-N-(2- hydroxy-ethyl)- benzamide | 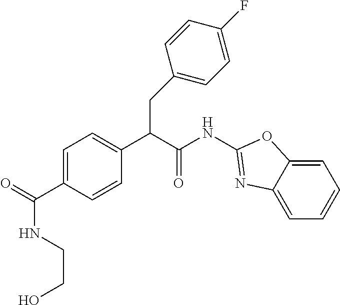 |
| I-34 | 4-[1- (Benzooxazol-2- ylcarbamoyl)-2-(4- fluoro-phenyl)- ethyl]-N-(2- dimethylamino- ethyl)-benzamide | 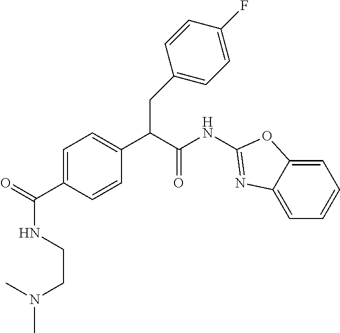 |
| I-35 | 3-(4-Fluoro- phenyl)-2-(4- methanesulfonyl- phenyl)-N-(4- methyl- benzooxazol-2-yl)- propionamide | 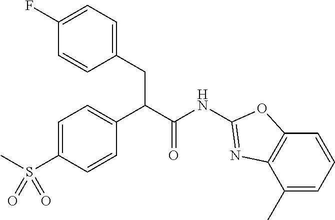 |
| I-36 | 3-(4-Fluoro- phenyl)-2-(4- methanesulfonyl- phenyl)-N-(6- methyl- benzooxazol-2-yl)- propionamide | 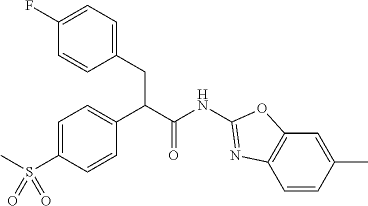 |
| I-37 | 3-(4-Fluoro- phenyl)-2-(4- methanesulfonyl- phenyl)-N-(5- methyl- benzooxazol-2-yl)- propionamide | 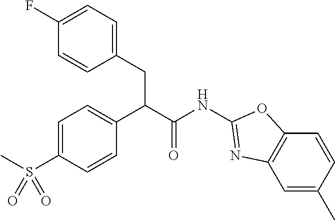 |
| I-38 | N-(6-Chloro- benzooxazol-2-yl)- 3-(4-fluoro- phenyl)-2-(4- methanesulfonyl- phenyl)- propionamide | 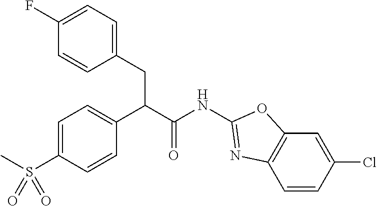 |
| I-39 | N-(5-Chloro- benzooxazol-2-yl)- 3-(4-fluoro- phenyl)-2-(4- methanesulfonyl- phenyl)- propionamide | 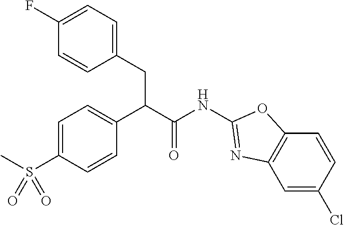 |
| I-40 | N-(6-Fluoro- benzooxazol-2-yl)- 3-(4-fluoro- phenyl)-2-(4- methanesulfonyl- phenyl)- propionamide | 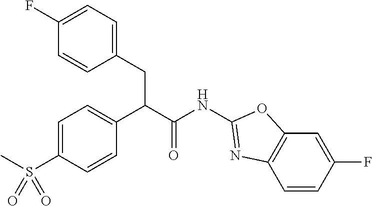 |
| I-41 | 3-(4-Fluoro- phenyl)-2-(4- methanesulfonyl- phenyl)-N-(5- trifluoromethyl- benzooxazol-2-yl)- propionamide | 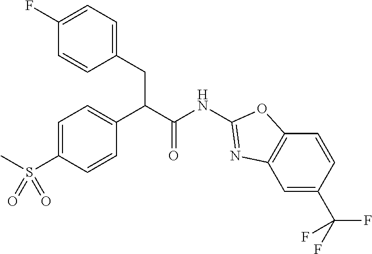 |
| I-42 | 3-(4-Fluoro- phenyl)-2-(4- methanesulfonyl- phenyl)-N-(7- methyl- benzooxazol-2-yl)- propionamide | 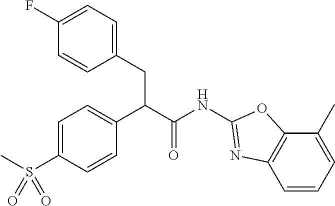 |
| I-43 | N-(5,7-Dimethyl- benzooxazol-2-yl)- 3-(4-fluoro- phenyl)-2-(4- methanesulfonyl- phenyl)- propionamide | 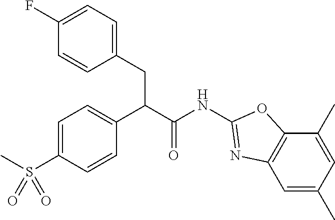 |
| I-44 | 3-(4-Fluoro- phenyl)-2-(4- methanesulfonyl- phenyl)-N-(5- methoxy- benzooxazol-2-yl)- propionamide | 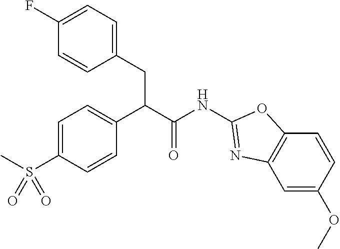 |
| I-45 | 2-[3-(4-Fluoro- phenyl)-2-(4- methanesulfonyl- phenyl)- propionylamino]- benzooxazole-6- carboxylic acid methyl ester | 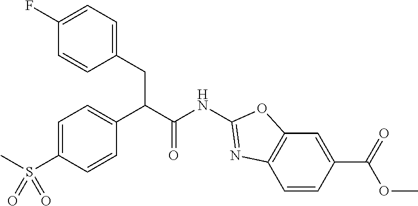 |
| I-46 | N-(5-Fluoro- benzooxazol-2-yl)- 3-(4-fluoro- phenyl)-2-(4- methanesulfonyl- phenyl)- propionamide | 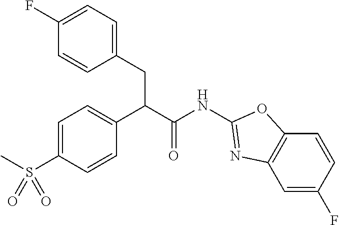 |
| I-47 | 3-(4-Fluoro- phenyl)-2-(4- methanesulfonyl- phenyl)-N-[5- (pyrrolidine-1- sulfonyl)- benzooxazol-2-yl]- propionamide | 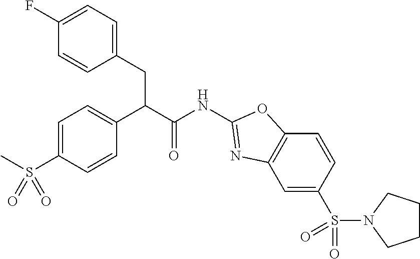 |
| I-48 | 3-(4-Fluoro- phenyl)-2-(4- methanesulfonyl- phenyl)-N-(5- phenyl-oxazol-2- yl)-propionamide | 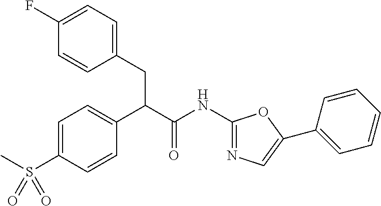 |
| I-49 | Acetic acid 5-[3- (4-fluoro-phenyl)- 2-(4- methanesulfonyl- phenyl)- propionylamino]- pyrazin-2-ylmethyl ester | 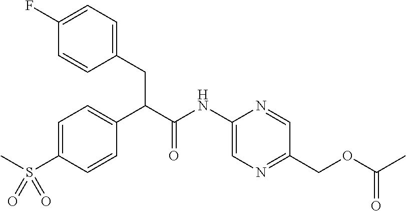 |
| I-50a | (R)-3-(4-Fluoro- phenyl)-N-(5- hydroxymethyl- pyrazin-2-yl)-2-(4- methanesulfonyl- phenyl)- propionamide | 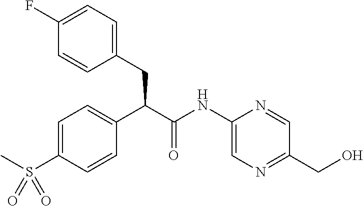 |
| I-50b | (S)-3-(4-Fluoro- phenyl)-N-(5- hydroxymethyl- pyrazin-2-yl)-2-(4- methanesulfonyl- phenyl)- propionamide | 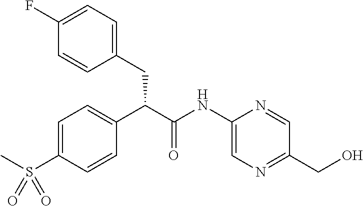 |
| I-51 | 3-(4-Fluoro- phenyl)-2-(4- methanesulfonyl- phenyl)-N- oxazolo[4,5- c]pyridin-2-yl- propionamide | 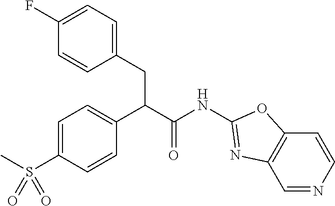 |
| I-52 | 3-(4-Fluoro- phenyl)-2-(4- methanesulfonyl- phenyl)-N- oxazolo [5,4- c]pyridin-2-yl- propionamide | 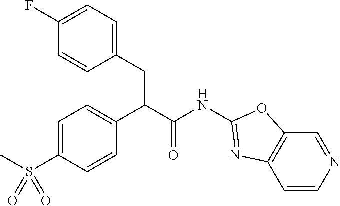 |
| I-53 | N-Benzooxazol-2- yl-2-(4-fluoro- phenyl)-3-(4- methanesulfonyl- phenyl)- propionamide | 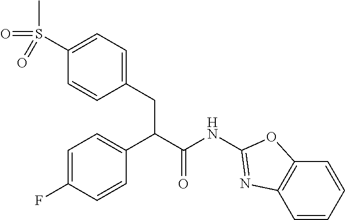 |
| I-54 | N-Benzooxazol-2- yl-3-(4-fluoro- phenyl)-2-pyridin- 4-yl-propionamide | 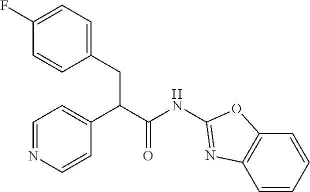 |
| I-55 | N-Benzooxazol-2- yl-2-(4- methanesulfonyl- phenyl)-3-pyridin- 4-yl-propionamide | 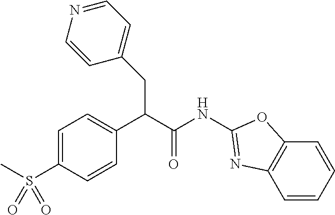 |

### Synthesis

### General Schemes.

Method 1

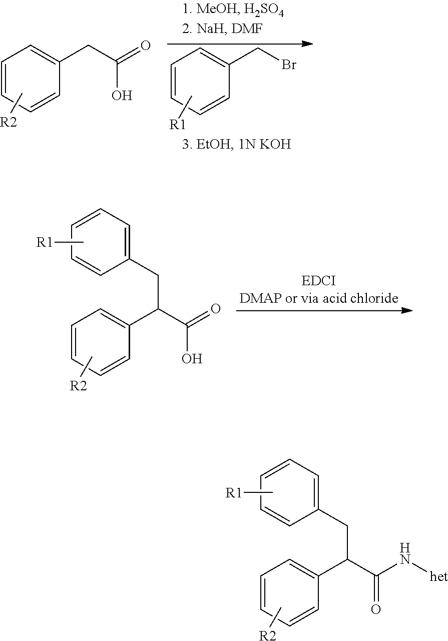

Method 2

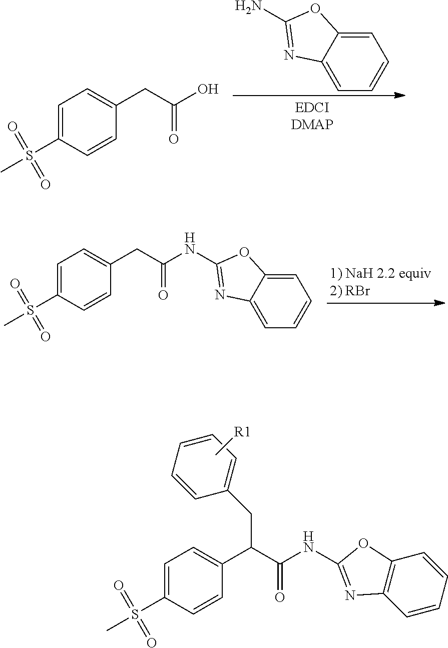

Method 3

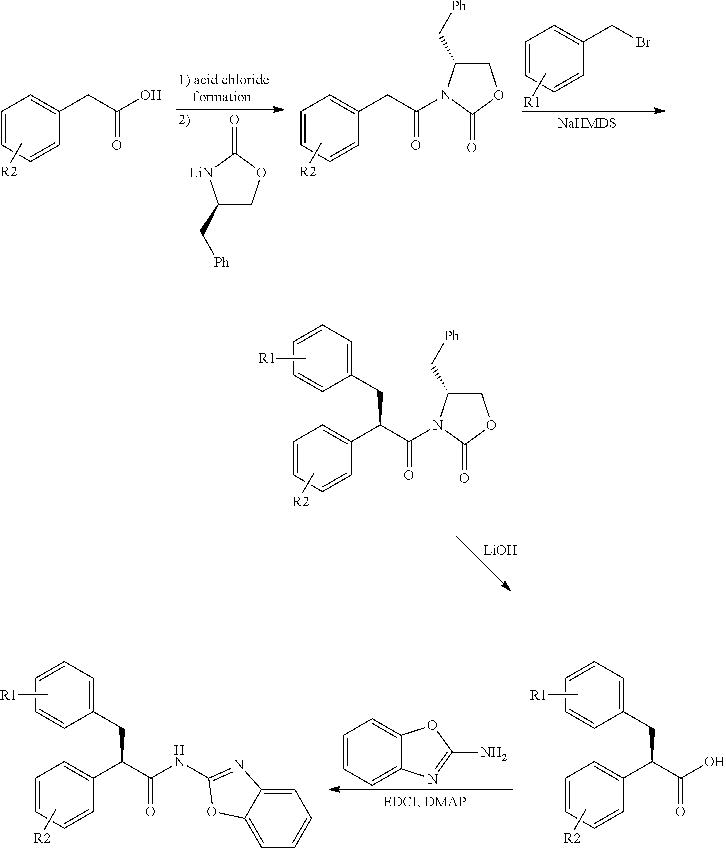

In the above schemes, each R¹ can be independently halo, hydroxy, lower alkyl, lower alkoxy, lower hydroxyalkyl, or lower haloalkyl, each R² can be independently halo, lower alkyl, lower hydroxyalkyl, lower alkoxy, C(═O)OR^{2′}, C(═O)N(R^{2′})₂, or S(═O)₂R^{2′}, wherein each R^{2′} can be independently H, lower alkyl, lower alkoxy, amino, or lower hydroxyalkyl.

Aminobenzoxazole Synthesis

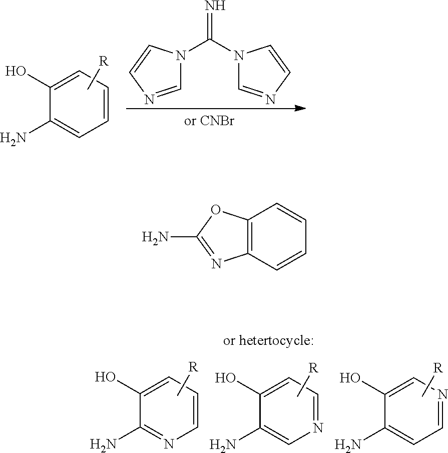

In the above scheme, R can be —OR′, alkyl, di-alkyl, —CO₂R, wherein gR′ is alkyl; for example R═OCH₃, —CO₂CH₃, F, —CF₃, or di-Me.

Pyrazine syntheses

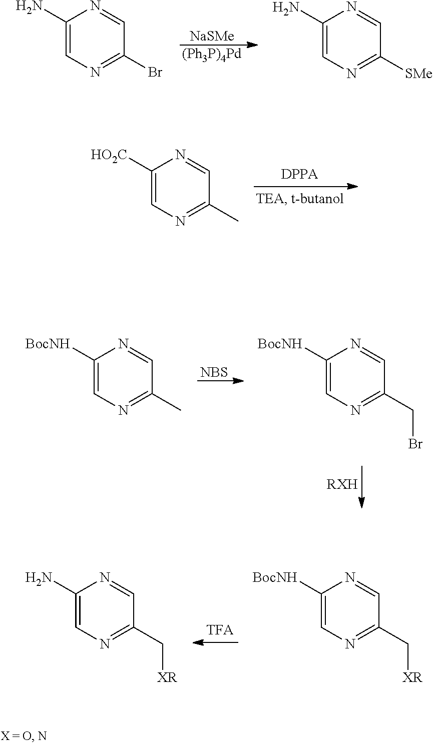

In the above scheme, XR can be OR or NRR′, wherein each R and R′ can independently be Alkyl or acyl, for example: XR can be —OCH₃, OEt, —N(CH₃)CH₂CH₂OCH₃, —N(CH₃)CH₂Ph, or —OAc.

### Pharmaceutical Compositions and Administration

### Dosage and Administration.

The compounds of the present invention may be formulated in a wide variety of oral administration dosage forms and carriers. Oral administration can be in the form of tablets, coated tablets, dragées, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Formulations

Pharmaceutical preparations for delivery by various routes are formulated as shown in the following Tables. “Active ingredient” or “Active compound” as used in the Tables means one or more of the Compounds of Formula I.

### Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

### Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

### Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

### Parenteral Formulation

| Ingredient | % wt./wt. |
|---|---|
| Active ingredient | 0.25 g |
| Sodium Chloride | qs to make isotonic |
| Water for injection | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

### Topical Formulation

| Ingredients | Grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

### Indications and Methods of Treatment

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

The compounds of the invention are also expected to find utility as analgesics in the treatment of diseases and conditions associated with pain from a wide variety of causes, including, but not limited to, inflammatory pain such as pain associated with arthritis (including rheumatoid arthritis and osteoarthritis), surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injuries (including fractures and sports injuries), and pain associated with functional bowel disorders such as irritable bowel syndrome.

Further, compounds of the invention are useful for treating respiratory disorders, including chronic obstructive pulmonary disorder (COPD), asthma, bronchospasm, and the like.

Additionally, compounds of the invention are useful for treating gastrointestinal disorders, including Irritable Bowel Syndrome (IBS), Inflammatory Bowel Disease (IBD), biliary colic and other biliary disorders, renal colic, diarrhea-dominant IBS, pain associated with GI distension, and the like.

The compounds of the invention are also useful for the treatment of muscular sclerosis and diabetes.

The application provides a method for treating arthritis, said method comprising administering to a subject in need thereof a therapeutically effective amount of a compound of Formula I or Formula II.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of Formula I or Formula II.

The application provides a method for treating a respiratory disorder selected from chronic obstructive pulmonary disorder (COPD), asthma, and bronchospasm, said method comprising administering to a subject in need thereof a therapeutically effective amount of a compound of Formula I or Formula II.

The application provides a method for treating diabetes, said method comprising administering to a subject in need thereof a therapeutically effective amount of a compound of Formula I or Formula II.

### EXAMPLES

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. The following abbreviations may be used in the Preparations and Examples.

### Abbreviations.

AIBN 2,2′ azobis(2-methylpropionitrile)
BETBDMS 2-bromoethoxy tertbutyldimethylsilane
BOP (benzotriazol-1-yloxy)tris(dimethylamino)phosphonium hexafluorophosphate
DBU 1,8-diazabicyclo[5.4.0]undec-7-ene
DCM dichloromethane/methylene chloride
DIEA diisopropylethylamine
DIPEA diisopropyl ethylamine (Hunig's base)
DME 1,2-dimethoxyethane (glyme)
DMF N,N-dimethylformamide
DMFDMA N,N-dimethylformamide dimethyl acetal
DMSO dimethyl sulfoxide
DMAP 4-dimethylaminopyridine
DPPA Diphenyl phosphorylazide
dppf 1,1′-Bis(diphenylphosphino)ferrocene
EDCI 1-ethyl-3-(3′-dimethylaminopropyl)carbodiimide
EDC.HCl N-ethyl-N′-(3-dimethylaminopropyl)carbodiimide hydrochloride
EtOAc ethyl acetate
EtOH ethanol
gc gas chromatography
HATU O-(7-Azabenzotriazol-1-yl)-N,N,N′,N′-tetramethyluronium hexafluorophosphate
HMPA hexamethylphosphoramide
HOAc acetic acid
HOAt 1-hydroxy-7-azabenzotriazole
HOBt N-Hydroxybenzotriazole
hplc high performance liquid chromatography
IPA isopropanol
IPBAPE isopropenylboronic acid pinacol ester
LAH lithium aluminum hydride
mCPBA m-chloroperbenzoic acid
MeCN acetonitrile
NaHMDS sodium hexamethyldisilazane
NBS N-bromosuccinimide
NCS N-chlorosuccinimide
NMM N-methyl morpholine
NMP N-methylpyrrolidinone
Pd₂(dba)₃ Tris(dibenzylideneacetone)dipalladium(0)
TBAF tetra-n-butyl ammonium fluoride
tBDMSICl tert-butyl-dimethylsilyl chloride
TEA triethylamine
TFA trifluoroacetic acid
THF tetrahydrofuran
LDA lithium diisopropylamine
TBDMS tert-butyl dimethylsilyl chloride
TLC thin layer chromatography
Xantphos 4,5-bis(diphenylphosphino)-9,9-dimethyl-xanthene

Compounds of the present invention can be prepared beginning with commercially available starting materials, unless where the source of the material is specifically stated, or utilizing general synthetic techniques and procedures known to those skilled in the art. Chemicals may be purchased from companies such as for example Aldrich, Argonaut Technologies, VWR, Lancaster, Princeton, Alfa, Oakwood, Strem, TCI, Fluorochem, Apollo, Matrix, Maybridge or Meinoah. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography. The intermediate and final compounds were named using the AutoNom2000 feature as part of the MDL ISIS Draw application. NMR data was collected using a Bruker NMR.

### Preparative Examples

### Example 1

### N-(5-Bromo-pyrazin-2-yl)-2-(4-methanesulfonylphenyl)-3-o-tolylpropionamide

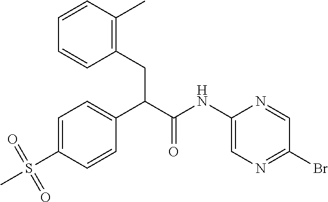

Step 1: A solution of 4-methylsulfonylphenylacetic acid (9.7 g, 45 mmol) in methanol (120 mL) and concentrated H₂SO₄ (0.45 mL) was warmed at 60° C. overnight. Reaction mixture was concentrated in vacuo, dissolved in CH₂Cl₂, and washed with saturated aqueous NaHCO₃ and then water. The organic phase was dried over Na₂SO₄ and concentrated in vacuo to afford a cream colored solid upon standing. Trituration with ether afforded (4-methanesulfonylphenyl)acetic acid methyl ester (8.8 g, 86%) as a white solid: ¹H NMR (300 MHz, CHLOROFORM-d) δ ppm 3.05 (s, 3H) 3.72 (s, 3H) 3.74 (s, 2H) 7.43-7.56 (m, 2H) 7.84-7.96 (m, 2H).

Step 2: To a slurry of sodium hydride (190 mg of 60% in oil dispersion, 4.75 mmol) in DMF (3 mL) under argon at 0° C. was added a solution of (4-methanesulfonylphenyl)-acetic acid methyl ester (902 mg, 3.94 mmol) in DMF (8 mL). The reaction mixture was stirred at room temperature for 50 minutes. 1-Bromomethyl-2-methyl-benzene (0.57 mL, 4.25 mmol) was added. The reaction mixture was stirred at room temperature for 2 h and then quenched with saturated NH₄Cl. The resultant mixture was poured onto water and extracted with 6:1 ether:CH₂Cl₂. A few drops of 1 N HCl was added and the mixture was extracted 3 times with 6:1 ether:CH₂Cl₂. The combined organic layer were dried over Na₂SO₄ and concentrated in vacuo. Purification by chromatography (silica, 0-40% ethyl acetate/hexanes gradient) afforded 2-(4-methanesulfonyl-phenyl)-3-o-tolyl-propionic acid methyl ester (940 mg, 72%): ¹H NMR (300 MHz, CHLOROFORM-d) δ ppm 2.26 (s, 3H) 2.92-3.12 (s, m, 4H) 3.35-3.53 (m, 1H) 3.64 (s, 3H) 3.94 (t, J=7.63 Hz, 1H) 6.91-7.00 (m, 1H) 7.00-7.19 (m, 3H) 7.49 (m, J=1.00 Hz, 2H) 7.88 (m, J=8.48 Hz, 2H), LC/MS-ESI observed [M−H]⁺ 331.

Step 3: To a suspension of 2-(4-methanesulfonyl-phenyl)-3-o-tolyl-propionic acid methyl ester (931 mg, 2.8 mmol) in ethanol (60 mL) was added 1 N aqueous KOH solution (6 mL). The reaction was stirred at room temperature overnight and then concentrated in vacuo. The residue was taken up in H₂O and CH₂Cl₂ and acidified with 1N aqueous HCl. The mixture was extracted 3 times with 0.01% methanol in CH₂Cl₂. The organic phase was dried over Na₂SO₄ and concentrated in vacuo to afford 2-(4-methanesulfonyl-phenyl)-3-o-tolyl-propionic acid (910 mg, 100%): ¹H NMR (300 MHz, CHLOROFORM-d) δ ppm 2.26 (s, 3H) 2.96-3.11 (s, m, 4H) 3.37-3.58 (m, 2H) 3.96 (t, J=7.54 Hz, 1H) 6.91-6.98 (m, 1H) 6.99-7.09 (m, 1H) 7.10-7.15 (m, 2H) 7.50 (d, J=8.48 Hz, 2H) 7.88 (d, J=8.29 Hz, 2H).

Step 4: 2-(4-Methanesulfonyl-phenyl)-3-o-tolyl-propionic acid (420 mg, 1.32 mmol) was dissolved in CH₂Cl₂ (10 mL) and placed under a N₂ atmosphere. DMF (6 drops) was added followed by oxalyl chloride (0.88 mL, 10 mmol). The reaction mixture was stirred at room temperature for 1 h, then concentrated in vacuo. To the crude residue was added toluene and the mixture was concentrated again in vacuo, then dissolved in THF (10 mL). To the resulting solution was added 2-amino-5-bromopyrazine (276 mg, 1.58 mmol) in THF (5 mL) and DIEA (0.69 mL, 4 mmol). The reaction mixture was stirred at room temperature for 4 h. The reaction mixture was poured onto water and extracted into ether/CH₂H₂, then extracted twice more with CH₂Cl₂. The combined organic extracts were dried over Na₂SO₄ and concentrated in vacuo. Purification by chromatography (silica, 25-50% ethyl acetate/hexanes gradient) afforded N-(5-bromopyrazin-2-yl)-2-(4-methanesulfonylphenyl)-3-o-tolyl-propionamide (161 mg, 81%) as a cream solid: m.p. 193.5-194.8° C., LC/MS-ESI observed [M+H]⁺ 474, 476.

### Example 2

### N-Benzooxazol-2-yl-2-(4-methanesulfonyl-phenyl)-3-o-tolyl-propionamide

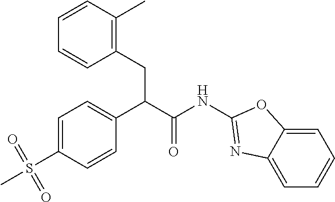

To a solution of 2-(4-methanesulfonylphenyl)-3-o-tolyl-propionic acid (prepared in example 1, 198 mg, 0.62 mmol) and 2-aminobenzoxazole (101 mg, 0.75 mmol) in CH₂Cl₂ (8 mL) was added DMAP (23 mg, 0.19 mmol) and EDC.HCl (144 mg, 0.75 mmol). The reaction mixture was stirred at room temperature overnight. Purification by chromatography (silica, 0.5-2.5% methanol/CH₂Cl₂ gradient) afforded a white solid. The solid was taken up in CH₂Cl₂ and poured onto 0.1% aqueous HCl and extracted 3 times with CH₂Cl₂ to remove the last trace of impurity. The organic layer was dried over Na₂SO₄ and concentrated in vacuo to afford N-benzooxazol-2-yl-2-(4-methanesulfonyl-phenyl)-3-tolylpropionamide (241 mg, 90%) of a white solid: m.p. 105.7-108.9° C., LC/MS-ESI observed [M+H]⁺ 435.

### Example 3

### N-(5-Chloro-benzooxazol-2-yl)-2-(4-methanesulfonylphenyl)-3-o-tolyl-propionamide

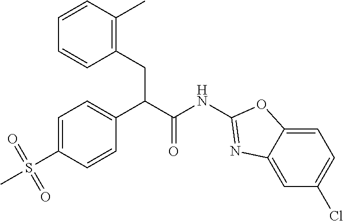

Step 1: To a slurry of sodium hydride (311 mg of 60% in oil dispersion, 7.79 mmol) in DMF (5 mL) under argon at 0° C. was added a solution of (4-methanesulfonylphenyl)-acetic acid methyl ester (prepared in example 1, 1.48 g, 6.49 mmol) in DMF (14 mL). The reaction mixture was stirred at room temperature for 50 minutes. 1-Bromomethyl-2-methyl-benzene (0.96 mL, 7.14 mmol) was added. The reaction mixture was stirred at room temperature for 2 h and then quenched with saturated NH₄Cl. The resultant mixture was poured onto water and extracted 4 times with ether. The combined organic layer were washed with saturated NaCl, dried over Na₂SO₄ and concentrated in vacuo. Purification by chromatography (silica, 0-30% ethyl acetate/hexanes gradient) afforded 2-(4-methanesulfonylphenyl)-3-o-tolyl-propionic acid methyl ester (1 g, 46%).

Step 2: To a suspension of 2-(4-methanesulfonylphenyl)-3-o-tolyl-propionic acid methyl ester (1 g, 3 mmol) in ethanol (80 mL) was added 1 N aqueous KOH solution (8 mL). The reaction was stirred at room temperature overnight and then concentrated in vacuo. The residue was taken up in H₂O and CH₂Cl₂ and acidified with 1N aqueous HCl. The mixture was extracted 4 times with CH₂Cl₂. The organic phase was dried over Na₂SO₄ and concentrated in vacuo to afford 2-(4-methanesulfonylphenyl)-3-o-tolyl-propionic acid (920 mg, 96%): ¹H NMR (300 MHz, CHLOROFORM-d) δ ppm 2.11 (s, 3H) 2.87-3.23 (s, m, 4H) 3.53 (dd, J=13.94, 6.03 Hz, 1H) 3.90 (dd, J=8.19, 6.12 Hz, 1H) 6.81-7.12 (m, 4H) 7.44 (d, J=8.29 Hz, 2H) 7.86 (d, J=8.29 Hz, 2H).

Step 3: To a solution of 2-(4-methanesulfonylphenyl)-3-o-tolyl-propionic acid (166 mg, 0.52 mmol) and 2-amino-5-chlorobenzoxazole (106 mg, 0.63 mmol) in CH₂Cl₂ (15 mL) was added DMAP (19 mg, 0.156 mmol) and EDC.HCl (121 mg, 0.63 mmol). The reaction mixture was stirred at room temperature for 3 days. The reaction mixture was concentrated in vacuo. Purification by chromatography (silica, 0.5-2. % methanol/CH₂Cl₂ gradient), followed by trituration with ether afforded N-(5-chloro-benzooxazol-2-yl)-2-(4-methanesulfonylphenyl)-3-o-tolyl-propionamide (86 mg, 35%) as a white solid: m.p. 184-185° C., LC/MS-ESI observed [M+H]⁺ 469.

### Example 4

### N-Benzooxazol-2-yl-2-(4-methanesulfonylphenyl)-3-phenylpropionamide

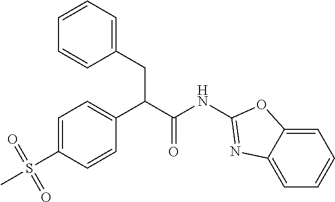

Step 1: To a solution of 4-methylsulfonylphenylacetic acid (214 mg, 1 mmol) and 2-amino-benzoxazole (147 mg, 1.1 mmol) in CH₂Cl₂ (10 mL) was added DMAP (37 mg, 0.3 mmol) and EDC.HCl (230 mg, 1.2 mmol). The reaction mixture was stirred at room temperature for 6 h. The reaction mixture was concentrated in vacuo. Purification by chromatography (silica, 10-75% ethyl acetate/hexanes gradient) afforded N-benzooxazol-2-yl-2-(4-methanesulfonyl-phenyl)acetamide (175 mg, 53%): LC/MS-ESI observed [M+H]⁺ 331.

Step 2: To a slurry of sodium hydride (36 mg of 60% in oil dispersion, 0.89 mmol) in DMF under argon at 0° C. was added a solution of N-benzooxazol-2-yl-2-(4-methanesulfonyl-phenyl)acetamide (123 mg, 0.37 mmol) in DMF (6 mL). The reaction mixture was stirred at room temperature for 1 h. Benzyl bromide (47.5 μL, 0.4 mmol) was added. The reaction mixture was stirred at room temperature for 2 h and then quenched with saturated aqueous NH₄Cl. The resultant mixture was poured onto 0.1% aqueous HCl and extracted 4 times with ether. The combined organic layer were washed with aqueous saturated NaCl, dried over Na₂SO₄ and concentrated in vacuo. Purification by chromatography (silica, 0-3% methanol/CH₂Cl₂ gradient) followed by re-purification by chromatography (silica, 20-60% ethyl acetate/hexanes gradient) afforded N-benzooxazol-2-yl-2-(4-methanesulfonylphenyl)-3-phenylpropionamide (48.7 mg, 31%): m.p. 118-119° C., LC/MS-ESI observed [M+H]⁺ 421.

### Example 5

### N-Benzooxazol-2-yl-3-(2-chlorophenyl)-2-(4-methanesulfonylphenyl)-propionamide

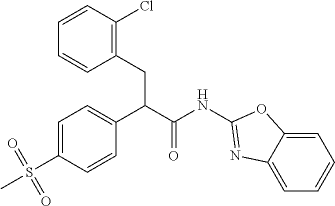

Step 1: To a solution of 4-methylsulfonylphenylacetic acid (2.03, 9.5 mmol) and 2-amino-benzoxazole (1.35 g, 10.13 mmol) in CH₂Cl₂ (100 mL) was added DMAP (343 mg, 2.8 mmol) and EDC.HCl (2.1 g, 11 mmol). The reaction mixture was stirred at room temperature overnight. The reaction mixture was concentrated in vacuo. Purification by chromatography (silica, 20-60% ethyl acetate/hexanes gradient, then 0-5% methanol in CH₂Cl₂ gradient) followed by trituration afforded N-benzooxazol-2-yl-2-(4-methanesulfonyl-phenyl)acetamide (1.64 g, 52%): ¹H NMR (300 MHz, DMSO-d₆) δ ppm 3.15 (s, 3H) 3.96 (s, 2H) 7.14-7.29 (m, 2H) 7.46-7.60 (m, 4H) 7.80-7.88 (m, 2H) 11.91 (s, 1H).

Step 2: To a slurry of sodium hydride (59 mg of 60% in oil dispersion, 1.45 mmol) in DMF (2 mL) under argon at 0° C. was added a solution of N-benzooxazol-2-yl-2-(4-methanesulfonyl-phenyl)acetamide (200 mg, 0.61 mmol) in DMF (5 mL). The reaction mixture was stirred at room temperature for 40 minutes. 2-chlorobenzyl bromide (138 mg, 0.67 mmol) was added. The reaction mixture was stirred at room temperature for 1 h and then quenched with aqueous saturated NH₄Cl. The resultant mixture was poured onto 0.1% aqueous HCl and ether, neutralized with saturated aqueous NaHCO₃ and extracted 2 times with ether and once with CH₂Cl₂. The combined organic layer were washed with saturated NaCl, dried over Na₂SO₄ and concentrated in vacuo. Purification by chromatography (silica, 0-2% methanol/CH₂Cl₂ gradient) followed by re-purification by chromatography (silica, 20-60% ethyl acetate/hexanes gradient) afforded N-benzooxazol-2-yl-3-(2-chlorophenyl)-2-(4-methanesulfonylphenyl)-propionamide (98 mg, 35%) as a white foam: m.p. 107-108° C., LC/MS-ESI observed [M+H]⁺ 455.

### Example 6

### N-Benzooxazol-2-yl-3-(2-chloro-4-fluorophenyl)-2-(4-methanesulfonylphenyl)-propionamide

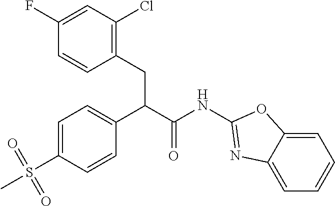

Preparation by a similar procedure to Example 5, except substituting 2-chloro-4-fluorobenzyl bromide for 2-chlorobenzyl bromide, afforded N-benzooxazol-2-yl-3-(2-chloro-4-fluoro-phenyl)-2-(4-methanesulfonyl-phenyl)-propionamide (55 mg, 19%); m.p. 127-128° C., LC/MS-ESI observed [M+H]⁺ 473.

### Example 7

### N-Benzooxazol-2-yl-3-(4-fluorophenyl)-2-(4-methanesulfonyl-phenyl)propionamide

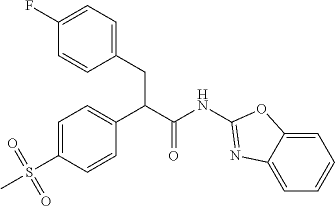

Preparation by a similar procedure to Example 5, except substituting 4-fluorobenzyl bromide for 2-chlorobenzyl bromide, afforded N-benzooxazol-2-yl-3-(4-fluorophenyl)-2-(4-methanesulfonyl-phenyl)-propionamide (72 mg, 27%) as a white solid: m.p. 109-110° C., LC/MS-ESI observed [M+H]⁺ 439.

### Example 8

### N-Benzooxazol-2-yl-3-(2-fluorophenyl)-2-(4-methanesulfonyl-phenyl)propionamide

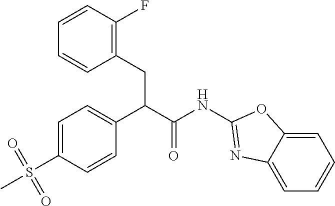

Preparation by a similar procedure to Example 5, except substituting 2-fluorobenzyl bromide for 2-chlorobenzyl bromide, afforded N-benzooxazol-2-yl-3-(2-fluorophenyl)-2-(4-methanesulfonyl-phenyl)-propionamide (46 mg, 18%) as a foam: m.p. 99-100° C., LC/MS-ESI observed [M+H]⁺ 439.

### Example 9

### N-Benzooxazol-2-yl-2-(4-methanesulfonylphenyl)-3-(4-methoxyphenyl)propionamide

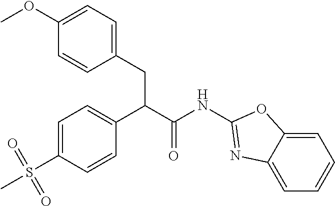

Preparation by a similar procedure to Example 5, except substituting 4-methoxybenzyl bromide for 2-chlorobenzyl bromide, afforded N-benzooxazol-2-yl-2-(4-methane-sulfonylphenyl)-3-(4-methoxyphenyl)propionamide (46 mg, 18%) as a foam: m.p. 101.9-105.9° C., LC/MS-ESI observed [M+H]⁺ 451.

### Example 10

### (S)—N-Benzooxazol-2-yl-2-(4-methanesulfonyl-phenyl)-3-o-tolyl-propionamide

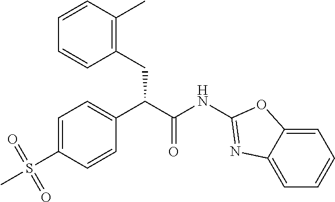

Step 1: To a solution of 4-methylsulfonylphenylacetic acid (3.72 g, 17.4 mmol) in CH₂Cl₂ (100 mL) was added 10 drops DMF and oxalyl chloride (4.4 mL, 50 mmol). The reaction mixture was stirred at room temperature for 2 h before concentration in vacuo. The residue was dissolved in 100 mL of THF to provide a 0.17 M solution of (4-methane-sulfonylphenyl)-acetyl chloride.

Step 2: To a solution of (S)(−)4-benzyl-2-oxazolidinone (1.63 g, 9.2 mmol) in dry THF (75 mL) under argon at −70° C. was added a solution of n-butyl lithium (3.7 mL of 2.5 M in hexanes, 9.2 mmol). The reaction mixture was stirred at room temperature for 30 minutes and recooled to −65° C. A solution of (4-methanesulfonylphenyl)acetyl chloride (50 mL of 0.17M in THF, 8.7 mmol) was added dropwise. The reaction mixture was stirred and gradually warmed from −65° C. to room temperature over 2 h. The reaction mixture was quenched with aqueous saturated NH₄Cl and poured onto ether, CH₂Cl₂ and 0.1% aqueous HCl. The mixture was extracted twice with ether/CH₂Cl₂ and once with CH₂Cl₂. The combined extracts were dried over Na₂SO₄ and concentrated in vacuo. Purification by sequential chromatography with (silica, 0-50% ethyl acetate/hexanes gradient) followed by (silica, 0-1% methanol/CH₂Cl₂ gradient) afforded (S)-4-benzyl-3-[2-(4-methanesulfonyl-phenyl)-acetyl]-oxazolidin-2-one (1.18 g, 36%).

Step 3: To a solution of (S)-4-benzyl-3-[2-(4-methanesulfonyl-phenyl)-acetyl]-oxazolidin-2-one (1.18 g, 3.16 mmol) and 1-bromomethyl-2-methyl-benzene (0.42 mL, 3.16 mmol) in dry THF (20 mL) at 0° C. under argon was added a solution of NaHMDS (694 mg, 3.79 mmol) in dry THF (5 mL). The reaction mixture was stirred at 0° C. for 40 minutes. The crude reaction mixture was quenched with saturated aqueous NH₄Cl, poured onto 5:1 ether:CH₂Cl₂ and 0.1% aqueous HCl and extracted twice into 5:1 Ether:CH₂Cl₂ and once into CH₂Cl₂. The combined organic extracts were washed with saturated NaCl, dried over Na₂SO₄ and concentrated in vacuo. Chromatography (silica, 10-30% ethyl acetate/hexanes gradient afforded (S)-4-Benzyl-3-[(R)-2-(4-methanesulfonyl-phenyl)-3-o-tolyl-propionyl]-oxazolidin-2-one (80 mg, 5%) as the minor isomer and (S)-4-benzyl-3-[(S)-2-(4-methanesulfonyl-phenyl)-3-o-tolyl-propionyl]oxazolidin-2-one (306 mg, 20%) as the major isomer¹: ¹H NMR (300 MHz, CHLOROFORM-d) δ ppm 2.38 (s, 3H) 2.60 (dd, J=13.56, 8.67 Hz, 1H) 2.95-3.12 (s, m, 5H) 3.57 (dd, J=13.85, 9.70 Hz, 1H) 4.05 (d, J=5.46 Hz, 2H) 4.56-4.67 (m, 1H) 5.61 (dd, J=9.61, 5.65 Hz, 1) 6.94 (dd, J=6.40, 3.01 Hz, 2H) 7.04-7.39 (m, 7H) 7.64 (d, J=8.29 Hz, 2H) 7.89 (d, J=8.48 Hz, 2H). ¹Diastereoselectivity assignment was based on literature precedent. (Heemstra, J. M.; Kerrigan, S. A.; Doerge, D. R.; Helferich, W. G.; Boulanger, W. A. Organic Letters 2006, 8, 5441.

Step 4: To solution of (S)-4-benzyl-3-[(S)-2-(4-methanesulfonyl-phenyl)-3-o-tolylpropionyl]-oxazolidin-2-one (306 mg, 0.64 mmol) in THF (30 mL) at 0° C. was added a 1N aqueous solution of LiOH (6 mL). The reaction mixture was stirred at 0° C. for 3 h and stirred at room temperature overnight. The crude reaction mixture was poured onto ether and H₂O and extracted with ether. The ether extract was washed once with 0.1% aqueous NaOH. The combined aqueous phase was acidified with aqueous HCl and extracted 3 times with CH₂Cl₂. The combined CH₂Cl₂ extracts were dried over Na₂SO₄ and concentrated in vacuo to afford (S)-2-(4-methanesulfonyl-phenyl)-3-o-tolyl-propionic acid (214 mg, 100%).

Step 5: To a solution of (S)-2-(4-methanesulfonyl-phenyl)-3-o-tolyl-propionic acid (214 mg, 0.67 mmol) and 2-amino-benzoxazole (90 mg, 0.67 mmol) in CH₂Cl₂ (10 mL) was added DMAP (24 mg, 0.2 mmol) and EDC.HCl (154 mg, 0.8 mmol). The reaction mixture was stirred at room temperature for 2 days. The reaction mixture was concentrated in vacuo. Purification by chromatography (silica, 0-1.5% methanol/CH₂Cl₂ gradient), followed by RPHPLC purification (C4 reverse phase, 10-90% acetonitrile/0.1% aqueous TFA buffer) afforded (S)—N-Benzooxazol-2-yl-2-(4-methanesulfonyl-phenyl)-3-o-tolyl-propionamide (68 mg, 23%): m.p. 106-108° C., LC/MS-ESI observed [M+H]⁺ 435, [α]²⁸=+43.6° (c=0.5, methanol), 75% ee by chiral HPLC determination.

### Example 11

### (R)—N-Benzooxazol-2-yl-2-(4-methanesulfonyl-phenyl)-3-o-tolyl-propionamide

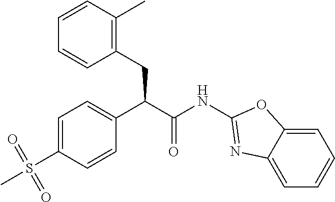

Preparation by a similar procedure to Example 10, except substituting (R)(+)4-benzyl-2-oxazolidinone for (S)(−)4-benzyl-2-oxazolidinone, afforded (S)—N-Benzooxazol-2-yl-2-(4-methanesulfonyl-phenyl)-3-o-tolyl-propionamide (50 mg): m.p. 99-101.5° C., LC/MS-ESI observed [M+H]⁺ 435, [α]²⁸=−28.6° (c=0.5, methanol), 50% ee by chiral HPLC determination.

### Example 12

### N-Benzooxazol-2-yl-3-(4-fluorophenyl)-2-(3-methanesulfonyl-phenyl)-propionamide

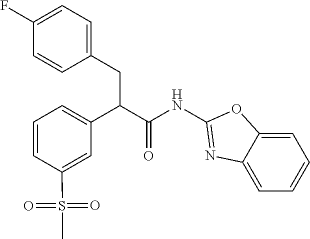

Step 1: A solution of 3-methylsulfonylphenylacetic acid (1 g, 4.67 mmol) in methanol (25 mL) and few drops of concentrated H₂SO₄ was warmed at 50° C. overnight. The reaction mixture was concentrated in vacuo, dissolved in CH₂Cl₂, poured onto H₂O and extracted 3 times with CH₂Cl₂. The organic phase was dried over Na₂SO₄ and concentrated in vacuo to afford (3-methanesulfonylphenyl)acetic acid methyl ester (1.04 g, 98%): ¹H NMR (300 MHz, CHLOROFORM-d) δ ppm 3.07 (s, 3H) 3.73 (s, 3H) 3.74 (s, 2H) 7.51-7.64 (m, 2H) 7.82-7.92 (m, 2H).

Step 2: To a slurry of sodium hydride (0.22 g of 60% in oil dispersion, 5.4 mmol) in DMF (8 mL) under argon at 0° C. was added a solution of (3-methanesulfonylphenyl)acetic acid methyl ester (1.03 g, 4.5 mmol) in DMF (8 mL). The reaction mixture was stirred at room temperature for 30 minutes. 4-fluorobenzyl bromide (0.59 mL, 4.72 mmol) was added. The reaction mixture was stirred at room temperature for 2 h and then quenched with saturated aqueous NH₄Cl. The resultant mixture was poured onto H₂O and was extracted 3 times with ether. The combined organic layer was washed with saturated aqueous NaCl, dried over Na₂SO₄ and concentrated in vacuo. Purification by chromatography (silica, 0% then 25% ethyl acetate/hexanes) afforded 3-(4-fluoro-phenyl)-2-(3-methanesulfonylphenyl)-propionic acid methyl ester (660 mg, 44%): ¹H NMR (300 MHz, CHLOROFORM-d) δ ppm 2.95-3.11 (s, m, 4H) 3.41 (dd, J=13.85, 8.38 Hz, 1H) 3.64 (s, 3H) 3.91 (dd, J=8.38, 7.44 Hz, 1H) 6.86-6.97 (m, 2H) 6.98-7.08 (m, 2H) 7.53 (t, J=7.72 Hz, 1H) 7.61 (dt, J=7.77, 1.58 Hz, 1H) 7.76-7.91 (m, 2H)

Step 3: To a mixture of 3-(4-fluoro-phenyl)-2-(3-methanesulfonylphenyl)-propionic acid methyl ester (660 mg, 1.96 mmol) in ethanol (25 mL) was added 1 N aqueous KOH solution (4.5 mL). The reaction was stirred at room temperature for 3 days and then concentrated in vacuo. The residue was taken up in H₂O and CH₂Cl₂ and acidified with 1N aqueous HCl. The mixture was extracted 3 times with 0.01% methanol in CH₂Cl₂. The organic phase was dried over Na₂SO₄ and concentrated in vacuo to afford 3-(4-fluoro-phenyl)-2-(3-methane-sulfonylphenyl)propionic acid (600 mg, 95%) as a white solid: ¹H NMR (300 MHz, CHLOROFORM-d) δ ppm 2.94-3.12 (s, m, 4H) 3.35-3.47 (m, 1H) 3.93 (t, J=7.82 Hz, 1H) 6.81-6.97 (m, 2H) 6.97-7.09 (m, 2H) 7.48-7.65 (m, 2H) 7.78-7.91 (m, 2H)

Step 4: To a solution of 3-(4-fluorophenyl)-2-(3-methanesulfonylphenyl)propionic acid (200 mg, 0.62 mmol) and 2-aminobenzoxazole (87 mg, 0.65 mmol) in CH₂Cl₂ (10 mL) was added DMAP (23 mg, 0.19 mmol) and EDC.HCl (144 mg, 0.75 mmol). The reaction mixture was stirred at room temperature overnight and then concentrated in vacuo. Purification by chromatography (silica, 0-50% ethyl acetate/hexanes stepwise gradient)) afforded N-benzooxazol-2-yl-3-(4-fluorophenyl)-2-(3-methanesulfonyl-phenyl)propionamide (62 mg, 23%): m.p. 99-100° C., LC/MS-ESI observed [M+H]⁺ 439.

### Example 13

### N-(5-Bromo-pyrazin-2-yl)-3-(4-fluorophenyl)-2-(4-methanesulfonylphenyl)-propionamide

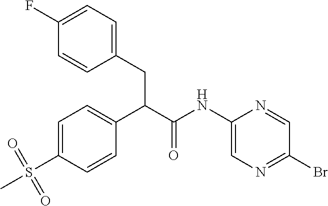

Step 1: To a solution of 4-methylsulfonylphenylacetic acid (5.0 g, 23.3 mmol) in tert-butanol (100 mL) was added DMAP (0.85 g, 7 mmol) and di-tert-butyl-dicarbonate. The reaction mixture was stirred at room temperature over night, concentrated in vacuo and purified by chromatography to afford (4-methanesulfonylphenyl)acetic acid tert-butyl ester (2.8 g, 45%): ¹H NMR (300 MHz, CHLOROFORM-d) δ ppm 1.45 (s, 9H) 3.05 (s, 3H) 3.63 (s, 2H) 7.48 (m, J=8.29 Hz, 2H) 7.90 (m, J=8.29 Hz, 2H).

Step 2: To a slurry of sodium hydride (143 mg of 60% in oil dispersion, 3.6 mmol) in DMF (5 mL) under argon at 0° C. was added a solution of (4-methanesulfonylphenyl)acetic acid tert-butyl ester (800 mg, 3.0 mmol) in DMF (8 mL). The reaction mixture was stirred at room temperature for 40 minutes. 4-fluorobenzyl bromide (0.39 mL, 3.15 mmol) was added. The reaction mixture was stirred at room temperature for 2 h and then quenched with saturated aqueous NH₄Cl. The resultant mixture was poured onto H₂O and was extracted 3 times with ether. The combined organic layer was washed with saturated aqueous NaCl, dried over Na₂SO₄ and concentrated in vacuo. Purification by chromatography (silica, 0%-25% ethyl acetate/hexanes gradient) afforded 3-(4-fluoro-phenyl)-2-(4-methanesulfonyl-phenyl)-propionic acid tert-butyl ester (495 mg, 44%).

Step 3: To a solution of 3-(4-fluoro-phenyl)-2-(4-methanesulfonyl-phenyl)-propionic acid tert-butyl ester (495 mg, 1.31 mmol) in CH₂Cl₂ (30 mL) was added TFA (10 mL). The reaction mixture was stirred at room temperature overnight and concentrated in vacuo. The resulting residue was slurried with ether/hexanes and concentrated in vacuo. to afford 3-(4-fluorophenyl)-2-(4-methanesulfonylphenyl)propionic acid (370 mg, 88%): ¹H NMR (300 MHz, DMSO-d₆) δ ppm 3.00 (dd, J=13.75, 7.35 Hz, 1H) 3.17 (br. S and H₂O, 3H) 3.25-3.46 (m, 1H) 4.06 (t, J=7.72 Hz, 1H) 6.92-7.13 (m, 3H) 7.23 (dd, J=8.67, 5.65 Hz, 2H) 7.59 (m, J=8.29 Hz, 2H) 7.87 (m, J=8.67 Hz, 2H)

Step 4: To a solution of 3-(4-fluorophenyl)-2-(4-methanesulfonylphenyl)propionic acid (365 mg, 1.13 mmol) and 2-amino-5-bromopyrazine (209 mg, 1.2 mmol) in CH₂Cl₂ (10 mL) was added DMAP (40 mg, 0.34 mmol) and EDC.HCl (260 mg, 1.35 mmol). The reaction mixture was stirred at room temperature overnight and then concentrated in vacuo. Purification by chromatography (silica, 0-50% ethyl acetate/hexanes stepwise gradient)) afforded N-(5-bromopyrazin-2-yl)-3-(4-fluorophenyl)-2-(4-methanesulfonyl-phenyl)-propionamide (156 mg, 29%): LC/MS-ESI observed [M+H]⁺ 478, 480.

### Example 14

### 3-(4-Fluorophenyl)-2-(4-methanesulfonyl-phenyl)-N-(5-methylsulfanyl-pyrazin-2-yl)-propionamide

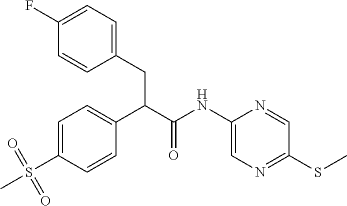

Step 1: A solution of 4-methylsulfonylphenylacetic acid (20 g, 93 mmol) in methanol (200 mL) and 3 drops of concentrated H₂SO₄ was stirred at room temperature overnight. An additional 6 drops of concentrated H₂SO₄ were added and stirring was continued at room temperature overnight. The reaction mixture was concentrated in vacuo to afford (4-methanesulfonylphenyl)acetic acid methyl ester (20.7 g, 98%).

Step 2: To a slurry of sodium hydride (7.25 g of 60% in oil dispersion, 109 mmol) in DMF (100 mL) under argon at 0° C. was added a solution of (4-methanesulfonylphenyl)-acetic acid methyl ester (20.7 g, 90.7 mmol) in DMF (400 mL) dropwise over 1 h. The reaction mixture was stirred at room temperature for 1.5 h. 4-fluorobenzyl bromide (12.3 mL, 99.6 mmol) was added. The reaction mixture was stirred at room temperature for 2 h and then quenched slowly with saturated aqueous NH₄Cl. The resultant mixture was acidified with 6N HCl and extracted 4 times with ether. The combined organic layer was washed with saturated NaCl, dried over Na₂SO₄ and concentrated in vacuo. Purification by chromatography (silica, 40% ethyl acetate/hexanes) afforded 3-(4-fluoro-phenyl)-2-(4-methanesulfonylphenyl)-propionic acid methyl ester (10.85 g, 36%).

Step 3: A mixture of 3-(4-fluoro-phenyl)-2-(4-methanesulfonylphenyl)-propionic acid methyl ester (10.79 g, 32 mmol) in ethanol (300 mL) and 1 N aqueous KOH solution (65 mL) was stirred at room temperature overnight and then concentrated in vacuo. The residue was taken up in 1% aqueous HCl (pH 1-2) and CH₂Cl₂. The mixture was extracted 3 times with CH₂Cl₂. The organic phase was dried over Na₂SO₄ and concentrated in vacuo to afford 3-(4-fluoro-phenyl)-2-(4-methanesulfonylphenyl)-propionic acid 9.8 g of a slightly impure solid. The solid was slurried in 5% aqueous NaOH. The aqueous solution was extracted with 3:1 ether:CH₂Cl₂. The organic phase was back-extracted with aqueous NaOH. The combined cloudy aqueous phase was extracted with CH₂Cl₂. Organic phase contained mostly impurity and was discarded. The aqueous phase was acidified with 10% aqueous HCl and extracted 6 times with CH₂Cl₂. Combined CH₂Cl₂ extracts were washed with water, dried over Na₂SO₄ and concentrated in vacuo to afford pure 3-(4-fluoro-phenyl)-2-(4-methanesulfonylphenyl)-propionic acid (8.6 g, 83%): ¹H NMR (300 MHz, DMSO-d₆) δ ppm 3.00 (dd, J=13.85, 7.44 Hz, 1H) 3.20 (s, 3H) 3.26-3.33 (m, 1H) 4.06 (t, J=7.91 Hz, 1H) 7.06 (t, J=8.85 Hz, 2H) 7.18-7.28 (m, 2H) 7.59 (m, J=8.48 Hz, 2H) 7.87 (m, J=8.29 Hz, 2H) 12.65 (s, 1H).

Step 4: A solution of 2-amino-5-bromopyrazine (600 mg, 3.45 mmol), tetrakis(triphenyl-phospine)palladium(0) (186 mg, 0.17 mmol) and NaSCH₃ (490 mg, 7 mmol) in DMF (20 mL) was warmed at 60° C. overnight in a sealed tube. The reaction mixture was poured onto water and aqueous NaHCO₃ and extracted 3 times into CH₂Cl₂/ether. The combined organic phase was washed with saturated NaCl, dried over Na₂SO₄ and concentrated in vacuo. Purification by chromatography (silica, 0-20% ethyl acetate/hexanes gradient) afforded 5-methylsulfanyl-pyrazin-2-ylamine (305 mg, 63%): ¹H NMR (300 MHz, CHLOROFORM-d) δ ppm 2.52 (s, 3H) 4.48 (br. s., 2H) 7.91 (d, J=1.51 Hz, 1H) 7.98 (d, J=1.51 Hz, 1H).

Step 5: To a solution of 3-(4-fluorophenyl)-2-(4-methanesulfonylphenyl)propionic acid (413 mg, 1.28 mmol) and 5-methylsulfanyl-pyrazin-2-ylamine (190 mg, 1.34 mmol) in CH₂Cl₂ (10 mL) was added DMAP (47 mg, 0.38 mmol) and EDC.HCl (294 mg, 1.53 mmol). The reaction mixture was stirred at room temperature overnight and then concentrated in vacuo. Purification by chromatography (silica, 0-40% ethyl acetate/hexanes stepwise gradient), followed by re-purification of the mixed fractions by chromatography (0-25% ethyl acetate/hexanes stepwise gradient) afforded 3-(4-fluorophenyl)-2-(4-methanesulfonyl-phenyl)-N-(5-methylsulfanyl-pyrazin-2-yl)-propionamide (355 mg, 62%): m.p. 187.7-188.4° C., LC/MS-ESI observed [M+H]⁺ 446.

### Example 15

### 5-[3-(4-Fluoro-phenyl)-2-(4-methanesulfonyl-phenyl)-propionylamino]-pyrazine-2-carboxylic acid ethyl ester

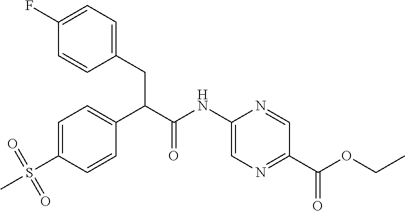

To a suspension of a mixture of 5-amino-pyrazine-2-carboxylic acid ethyl ester and 5-amino-pyrazine-2-carboxylic acid methyl ester (approximately 2:1) in CH₂Cl₂ (40 mL) was added DIEA (1.04 mL, 6 mmol) and DMF (5 mL). To the resulting suspension was added 3-(4-fluorophenyl)-2-(4-methanesulfonylphenyl)propionic acid (prepared as described in example 14, 1.5 g, 4.7 mmol), DMAP (0.17 g, 1.4 mmol) and EDC.HCl (1.08 g, 5.6 mmol) followed by additional CH₂Cl₂ (10 mL) and DMF (6 mL). The resulting solution was stirred at room temperature overnight. The reaction mixture was poured onto 0.01% aqueous HCl and extracted 3 times into CH₂Cl₂. The combined organic layer was dried over Na₂SO₄ and concentrated in vacuo. Purification by chromatography (silica, 30%-50% ethyl acetate/hexanes stepwise gradient), followed by re-purification of the mixed fractions by chromatography (50% ethyl acetate/hexanes) afforded the front-running product, 5-[3-(4-fluorophenyl)-2-(4-methanesulfonylphenyl)-propionylamino]-pyrazine-2-carboxylic acid ethyl ester (438 mg, 20%): m.p. 99-100° C., LC/MS-ESI observed [M+H]⁺ 472; and the back-running product, 5-[3-(4-fluoro-phenyl)-2-(4-methanesulfonylphenyl)propionylamino]-pyrazine-2-carboxylic acid methyl ester (260 mg, 12%): m.p. 106-107° C., LC/MS-ESI observed [M+H]⁺ 458.

### Example 16

### 3-(4-Fluorophenyl)-N-(5-hydroxymethylpyrazin-2-yl)-2-(4-methanesulfonylphenyl)-propionamide

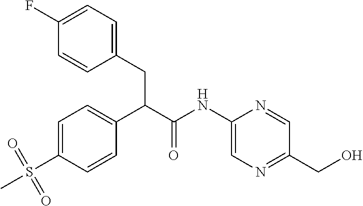

To a solution of 5-[3-(4-fluorophenyl)-2-(4-methanesulfonylphenyl)-propionylamino]-pyrazine-2-carboxylic acid methyl ester (prepared in example 15, 177 mg, 0.387 mmol) in methanol (6 mL) at 0° C. was added NaBH₄ (72 mg, 1.9 mmol) for 1 h. Additional NaBH₄ (70 mg, 1.8 mmol) was added and the reaction was stirred at room temperature for 30 minutes. The reaction progressed to 80% completion. The reaction mixture was concentrated in vacuo.

To a solution of 5-[3-(4-fluorophenyl)-2-(4-methanesulfonylphenyl)-propionylamino]-pyrazine-2-carboxylic acid ethyl ester (378 mg, 0.8 mmol) in methanol (12 mL) was added NaBH₄ (total amount 150 mg, 4 mmol) portion-wise until reaction did not progress further by LCMS. The reaction only progressed to 30% completion. Combined with above reduction and concentrated in vacuo. The residue was dissolved in CH₂Cl₂ and methanol. Silica was added and solvent was evaporated. Purification by chromatography afforded 3-(4-fluorophenyl)-N-(5-hydroxymethylpyrazin-2-yl)-2-(4-methanesulfonylphenyl)-propionamide (70 mg, 14%): LC/MS-ESI observed [M+H]⁺ 430.

### Example 17

### 3-(4-Fluoro-phenyl)-N-[5-(1-hydroxy-1-methyl-ethyl)-pyrazin-2-yl]-2-(4-methanesulfonylphenyl)-propionamide

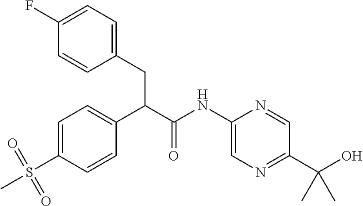

5-[3-(4-fluorophenyl)-2-(4-methanesulfonylphenyl)-propionylamino]-pyrazine-2-carboxylic acid ethyl ester was prepared using a similar procedure to that described in example 15 (512 mg, 24%).

To a solution of 5-[3-(4-fluorophenyl)-2-(4-methanesulfonylphenyl)-propionylamino]-pyrazine-2-carboxylic acid ethyl ester (181 mg, 0.37 mmol) in dry THF (3 mL) under argon at 0° C. was added methyl magnesium bromide (0.28 mL of 3M in ether, 0.83 mmol). The reaction mixture was stirred at 0-10° C. for 30 minutes and then the reaction was quenched with aqueous citric acid. The reaction mixture was poured onto H₂O, acidified with aqueous HCl and extracted 3 times with CH₂Cl₂. The organic layer was washed with brine, dried over Na₂SO₄ and concentrated in vacuo. Chromatography (silica, 30% ethyl acetate/hexanes; then 50% ethyl acetate/hexanes) afforded 3-(4-fluoro-phenyl)-N-[5-(1-hydroxy-1-methyl-ethyl)-pyrazin-2-yl]-2-(4-methanesulfonyl-phenyl)-propionamide (60 mg, 35%); LC/MS-ESI observed [M+H]⁺ 458.

### Example 18

### 3-(4-Fluoro-phenyl)-2-(4-methanesulfonyl-phenyl)-N-oxazolo[4,5-b]pyridin-2-yl-propionamide

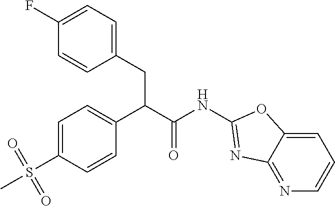

Step 1: To a solution of cyanogen bromide (4.5 g, 42 mmol) in H₂O (18 mL) was added 2-amino-3-hydroxypyridine (4.5 g, 40.9 mmol) and the resultant reaction mixture was stirred at reflux temperature 15 minutes and then cooled to room temperature. The reaction mixture was neutralized carefully with solid NaHCO₃ and diluted with water. The solid was collected, washed 5 times with H₂O and dried at 50° C. under vacuum to afford oxazolo[4,5-b]pyridin-2-ylamine (4.42 g, 80%): ¹H NMR (300 MHz, DMSO-d₆) δ ppm 6.95 (dd, J=7.82, 5.18 Hz, 1H) 7.65 (dd, J=7.82, 1.41 Hz, 1H) 7.91 (s, 2H) 8.08 (dd, J=5.18, 1.41 Hz, 1H).

Step 2: A mixture of 3-(4-fluorophenyl)-2-(4-methanesulfonylphenyl)propionic acid (prepared in example 14, 322 mg, 1.0 mmol), oxazolo[4,5-b]pyridin-2-ylamine (142 mg, 1.05 mmol), DMAP (37 mg, 0.3 mmol) and EDC.HCl (230 mg, 1.2 mmol) in 4:1 CH₂Cl₂:DMF (12 mL) was stirred at room temperature overnight and then concentrated in vacuo. Purification by chromatography (silica, 350:10:1 CH₂Cl₂:methanol:NH₄OH), followed by chromatography (silica 2.5% methanol/CH₂Cl₂) and subsequent trituration with ethanol afforded pure 3-(4-fluorophenyl)-2-(4-methanesulfonyl-phenyl)-N-oxazolo[4,5-b]pyridin-2-yl-propionamide (89 mg, 20%): m.p. 261-263.7° C., LC/MS-ESI observed [M+H]⁺ 440.

### Example 19

### 3-(4-Fluorophenyl)-2-(4-methanesulfonyl-phenyl)-N-(5-methylpyrazin-2-yl)-propionamide

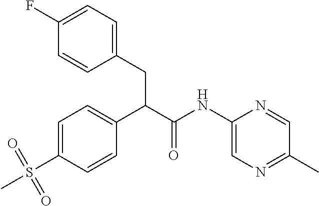

To a solution of 3-(4-fluorophenyl)-2-(4-methanesulfonylphenyl)propionic acid (prepared in example 14, 250 mg, 0.78 mmol) and 5-methyl-pyrazin-2-ylamine (90 mg, 0.82 mmol) in CH₂Cl₂ (20 mL) was added DMAP (29 mg, 0.23 mmol) and EDC.HCl (179 mg, 0.94 mmol). The reaction mixture was stirred at room temperature overnight and then concentrated in vacuo. Purification by chromatography (silica, 35% ethyl acetate/hexanes) afforded 3-(4-fluorophenyl)-2-(4-methanesulfonylphenyl)-N-(5-methylpyrazin-2-yl)-propionamide (175 mg, 54%): m.p. 172.4-175.3° C., LC/MS-ESI observed [M+H]⁺ 414.

### Example 20

### 3-(4-Fluoro-phenyl)-N-(6-hydroxymethyl-benzooxazol-2-yl)-2-(4-methanesulfonyl-phenyl)-propionamide

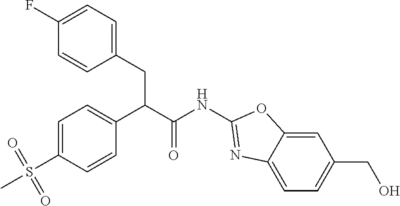

Step 1: To a solution of imidazole (6.8 g, 100 mmol) in CH₂Cl₂ (500 mL) was added cyanogen bromide (11 mL of 3M in CH₂Cl₂, 33 mmol) and the resultant mixture was heated under nitrogen at reflux for 30 minutes. The reaction mixture was cooled to room temperature and filtered. The filtrate was concentrated to 50 mL and crystallized in the freezer for 2 days. The solid was collected by filtration, washed with cold CH₂Cl₂ and dried under vacuum to afford (di-imidazol-1-yl)-methyleneamine (5.3 g, 99%).

Step 2: A mixture of methyl 4-amino-3-hydroxybenzoate (883 mg, 5.3 mmol) and (diimidazol-1-yl)-methyleneamine (851 mg, 5.3 mmol) in dry THF (40 mL) was warmed at 65° C. overnight. Additional (di-imidazol-1-yl)-methyleneamine (450 mg, 2.8 mmol) was added and the reaction was warmed at 75° C. for 3 days. The reaction mixture was poured onto H₂O and ethyl acetate and extracted 2 times with ethyl acetate. The organic phase was washed with saturated aqueous NH₄Cl, washed with saturated aqueous NaCl, dried over Na₂SO₄ and concentrated in vacuo to give a dark solid. This dark solid was dissolved in dry THF (30 mL) and the resulting solution was placed under argon and cooled to 0° C. A solution of LAH (5.2 mL of 2M in THF) was added. The reaction mixture was stirred at room temperature overnight, then quenched by sequential addition of H₂O (0.178 mL), a 15% aqueous solution of NaOH (0.178 mL) and H₂O (0.53 mL). After stirring at room temperature for 10 minutes, the mixture was poured onto 0.01% NaOH and CH₂Cl₂ (containing 0.5% methanol) and the mixture was extracted 4 times with CH₂Cl₂ (containing 0.5% methanol). The combined organic phase was washed with saturated aqueous NaCl, dried over Na₂SO₄ and concentrated in vacuo. The residue was slurried in approximately 1-5% methanol in CH₂Cl₂. The solid was collected, washed several times with CH₂Cl₂ to afford (2-aminobenzooxazol-6-yl)-methanol. (118 mg) as a yellow solid and the filtrate was purified by chromatography (silica, 4% methanol in CH₂Cl₂) to afford an additional 23 mg (16% overall yield): ¹H NMR (300 MHz, DMSO-d₆) δ ppm 4.50 (d, J=5.84 Hz, 2H) 5.13 (t, J=5.75 Hz, 1H) 7.00-7.18 (m, 2H) 7.26 (d, J=0.75 Hz, 1H) 7.32 (s, 2 H).

Step 3: A mixture of (2-aminobenzooxazol-6-yl)-methanol (133 mg, 0.8 mmol), t-butyl-dimethylsilyl chloride (133 mmg, 0.88 mmol) and imidazole (68 mg, 1 mmol) in 10:1 DMF:CH₂Cl₂ (15 mL) was stirred at room temperature for 4 h. Additional t-butyl-dimethylsilyl chloride (30 mg) was added and the reaction mixture was stirred at room temperature overnight. The reaction mixture was concentrated in vacuo, poured onto H₂O and CH₂Cl₂, and extracted 3 times with CH₂Cl₂. The combined organic extract was dried over Na₂SO₄ and concentrated in vacuo. Chromatography (silica, 30% ethyl acetate/hexanes afforded 6-(tert-butyl-dimethyl-silanyloxymethyl)benzooxazol-2-yl-amine (180 mg, 81%): ¹H NMR (300 MHz, CHLOROFORM-d) δ ppm 0.10 (s, 6H) 0.94 (s, 9H) 4.78 (s, 2H) 5.26 (br. s., 2H) 7.08-7.14 (m, 1H) 7.27-7.32 (m, 2H).

Step 4: To a solution of 3-(4-fluorophenyl)-2-(4-methanesulfonylphenyl)propionic acid (prepared in example 14, 187 mg, 0.58 mmol) and 6-(tert-butyl-dimethyl-silanyloxymethyl)-benzooxazol-2-yl-amine (178 mg, 0.64 mmol) in CH₂Cl₂ (10 mL) was added DMAP (21 mg, 0.17 mmol) and EDC.HCl (133 mg, 0.7 mmol). The reaction mixture was stirred at room temperature overnight and then concentrated in vacuo. Purification by chromatography (2% methanol in CH₂Cl₂) afforded N-[6-(tert-butyl-dimethyl-silanyloxymethyl)-benzooxazol-2-yl]-3-(4-fluorophenyl)-2-(4-methanesulfonylphenyl)-propionamide (293 mg). To a solution of N-[6-(tert-butyl-dimethyl-silanyloxymethyl)-benzooxazol-2-yl]-3-(4-fluorophenyl)-2-(4-methanesulfonylphenyl)-propionamide in THF (8 mL) was added a solution of TBAF (1 mL of 1M in THF) and the reaction was stirred at room temperature overnight and then the reaction was concentrated in vacuo. Purification by chromatography (silica 1.5% methanol in CH₂Cl₂) afforded 3-(4-fluoro-phenyl)-N-(6-hydroxymethylbenzooxazol-2-yl)-2-(4-methanesulfonylphenyl)-propionamide (176 mg, 65%): m.p. 128-130° C., LC/MS-ESI observed [M+H]⁺ 469.

### Example 21

### 3-(4-Fluorophenyl)-2-(4-methanesulfonylphenyl)-N-(5-methoxymethylpyrazin-2-yl)-propionamide

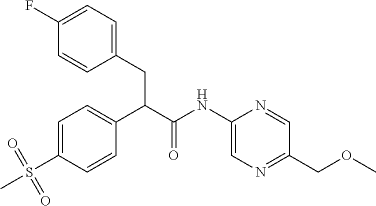

Step 1: To a solution of 2-methylpyrazine-5-carboxylic acid (10 g, 72.4 mmol) and TEA (20 mL, 108 mmol) in t-butanol (160 mL) and dioxanes (100 mL) was added diphenyl phosphorylazide (23.4 mL, 108 mmol) and the resulting solution was warmed at 100° C. for 6 hours and then cooled to room temperature overnight. The crude reaction mixture was concentrated in vacuo, dissolved in CH₂Cl₂ and methanol, poured onto water and extracted 3 times with CH₂Cl₂. The organic extract was dried over Na₂SO₄ and concentrated in vacuo. Purification by chromatography (silica 5:4:1 hexanes:CH₂Cl₂:ethyl acetate) following trituration with ethyl acetate/hexanes afforded (5-methyl-pyrazin-2-yl)-carbamic acid tert-butyl ester (6.65 g, 44%) as a white solid: ¹H NMR (300 MHz, CHLOROFORM-d) δ ppm 1.56 (s, 9 H) 2.51 (s, 3H) 7.88 (br. s., 1H) 8.10 (d, J=0.94 Hz, 1H) 9.17 (d, J=1.13 Hz, 1H).

Step 2: To a solution of (5-methyl-pyrazin-2-yl)-carbamic acid tert-butyl ester (6.64 g, 31.6 mmol) and AIBN (1 g, 6.3 mmol) in CCl₄ was added NBS (5.64 g, 31.7 mmol) and the reaction mixture was stirred at reflux for 6 h until reaction did not proceed any further (approximately 50% complete). The reaction mixture was filtered through celite, washed 3 times with CH₂Cl₂ and concentrated in vacuo. Purification by chromatography (silica, 4:5:1 CH₂Cl₂:hexanes:ethyl acetate) afforded (5-bromomethyl-pyrazin-2-yl)-carbamic acid tert-butyl ester (2.35 g, 26%) and recovered starting material (3.1 g): ¹H NMR (300 MHz, CHLOROFORM-d) δ ppm 1.57 (s, 9H) 4.55 (s, 2H) 8.16 (s, 1H) 8.34 (d, J=1.32 Hz, 1H) 9.28 (d, J=1.32 Hz, 1H).

Step 3: To a solution of (5-bromomethylpyrazin-2-yl)-carbamic acid tert-butyl ester (1.07 g, 3.7 mmol) in dry methanol (50 mL) was added K₂CO₃ (800 mg) and the reaction was stirred under nitrogen at room temperature for 15 minutes. The crude mixture was filtered through celite, washed with CH₂Cl₂ and methanol and the resulting combined filtrate and washes were concentrated in vacuo. The residue was taken up in CH₂Cl₂, poured onto aqueous NH₄Cl, adjusted to pH=8 and extracted twice with CH₂Cl₂. The organic extract was dried over Na₂SO₄ and concentrated in vacuo. Purification by chromatography (silica, 8:46:46 ethyl acetate:CH₂Cl₂:hexanes) afforded (5-methoxy-methyl-pyrazin-2-yl)-carbamic acid tert-butyl ester (440 mg, 50%): ¹H NMR (300 MHz, CHLOROFORM-d) δ ppm 1.57 (s, 9H) 3.47 (s, 3H) 4.58 (s, 2 H) 8.31 (br. s., 1H) 8.34-8.38 (m, 1H) 9.26 (d, J=1.32 Hz, 1H).

Step 4: A solution of 5-methoxy-methyl-pyrazin-2-yl)-carbamic acid tert-butyl ester (200 mg, 0.84 mmol) in CH₂Cl₂ (5 mL) was added TFA (1 mL) and the reaction mixture was stirred at room temperature for 3 h and then concentrated in vacuo. The residue was dissolved in CH₂Cl₂, poured onto saturated aqueous NaHCO₃ and extracted twice with CH₂Cl₂. The combined extracts were dried over Na₂SO₄ and concentrated in vacuo to afford 5-methoxymethyl-pyrazin-2-ylamine (82 mg, 70%). ¹H NMR (300 MHz, CHLOROFORM-d) δ ppm 3.45 (s, 3H) 4.46 (s, 2H) 4.64 (br. s., 1H) 7.97 (d, J=1.51 Hz, 1H) 8.06 (d, J=1.32 Hz, 1H).

Step 5: To a solution of 3-(4-fluorophenyl)-2-(4-methanesulfonylphenyl)propionic acid (prepared in example 14, 168 mg, 0.52 mmol) and 5-methoxymethyl-pyrazin-2-ylamine (80 mg, 0.57 mmol) in CH₂Cl₂ was added DMAP (19 mg, 0.156 mmol) and EDC.HCl (120 mg, 0.62 mmol). The reaction mixture was stirred at room temperature for 2 h and then concentrated in vacuo. Purification by chromatography twice (silica, 30-60% ethyl acetate/hexanes) afforded 3-(4-fluorophenyl)-2-(4-methanesulfonylphenyl)-N-(5-methoxymethylpyrazin-2-yl)-propionamide (72 mg, 31%): m.p. 69-82.3° C., LC/MS-ESI observed [M+H]⁺ 444.

### Example 22

### 3-(4-Fluorophenyl)-2-(4-methanesulfonylphenyl)-N-(5-ethoxymethylpyrazin-2-yl)-propionamide

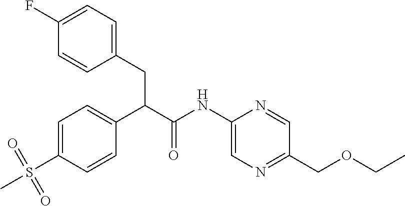

Preparation by a similar procedure to Example 21, except substituting ethanol for methanol in step 3, afforded 3-(4-fluorophenyl)-2-(4-methanesulfonylphenyl)-N-(5-ethoxymethylpyrazin-2-yl)-propionamide (27 mg) as a foam: m.p. 99-100° C., ¹H NMR (300 MHz, CHLOROFORM-d) δ ppm 1.26 (t, J=6.97 Hz, 3H) 3.05 (s, m 4H) 3.62 (q, J=6.97 Hz, 2 H) 3.87 (t, J=7.44 Hz, 1H) 4.61 (s, 2H) 6.88-6.95 (m, 2H) 7.08 (dd, J=8.76, 5.37 Hz, 2H) 7.55 (d, J=8.48 Hz, 2H) 7.90 (d, J=8.48 Hz, 2H) 7.94 (s, 1H) 8.25-8.35 (m, 1H) 9.43 (s, 1H), LC/MS-ESI observed [M+H]⁺ 457.

### Example 23

### 3-(4-Fluoro-phenyl)-2-(4-methanesulfonyl-phenyl)-N-(5-{[(2-methoxy-ethyl)-methyl-amino]-methyl}-pyrazin-2-yl)-propionamide

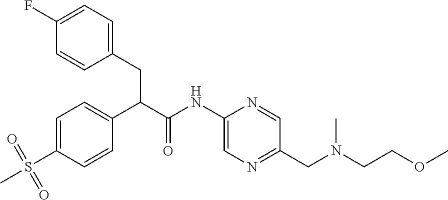

Step 1: To a solution of (5-bromomethylpyrazin-2-yl)-carbamic acid tert-butyl ester (prepared in example 21, 0.449 g, 1.56 mmol) and (2-methoxyethyl)-methylamine (0.23 mL, 2.1 mmol) in dry acetonitrile (10 mL) was added K₂CO₃ (800 mg) and the reaction was stirred under nitrogen at 70° C. in a sealed tube overnight. The crude mixture was diluted with ether, poured onto H₂O and extracted 2 times into ether and once with CH₂Cl₂. Combined organic layer was washed with saturated aqueous NaCl, dried over Na₂SO₄ and concentrated in vacuo. Purification by chromatography (silica, 2.5% methanol in CH₂Cl₂) afforded (5-{[(2-methoxy-ethyl)-methyl-amino]-methyl}pyrazin-2-yl) carbamic acid tert-butyl ester (383 mg, 83%) as a white solid: ¹H NMR (300 MHz, CHLOROFORM-d) δ ppm 1.55 (s, 9H) 2.32 (s, 3H) 2.67 (t, J=5.75 Hz, 2H) 3.34 (s, 3H) 3.53 (t, J=5.65 Hz, 2H) 3.72 (s, 2H) 7.82 (s, 1H) 8.31 (d, J=1.51 Hz, 1H) 9.21 (d, J=1.32 Hz, 1H).

Step 2: A solution of (5-{[(2-methoxy-ethyl)-methyl-amino]-methyl}pyrazin-2-yl) carbamic acid tert-butyl ester (382 mg, 1.3 mmol) in CH₂Cl₂ (40 mL) was added TFA (4 mL) and the reaction mixture was stirred at room temperature for 2 h and then concentrated in vacuo. The residue was dissolved in CH₂Cl₂, poured onto saturated aqueous NaHCO₃ and extracted 3 times into CH₂Cl₂. The combined extracts were dried over Na₂SO₄ and concentrated in vacuo to afford 5-{[(2-methoxyethyl)-methylamino]-methyl}-pyrazin-2-ylamine (98 mg, 38%): ¹H NMR (300 MHz, CHLOROFORM-d) δ ppm 2.33 (s, 3H) 2.67 (t, J=5.75 Hz, 2H) 3.35 (s, 3H) 3.54 (t, J=5.75 Hz, 2H) 3.63 (s, 2H) 4.55 (br. s., 2H) 7.97 (d, J=1.32 Hz, 1H) 8.04 (d, J=1.51 Hz, 1H).

Step 3: To a solution of 3-(4-fluorophenyl)-2-(4-methanesulfonylphenyl)propionic acid (prepared in example 14, 160 mg, 0.5 mmol) and 5-{[(2-methoxyethyl)-methylamino]-methyl}-pyrazin-2-ylamine (98 mg, 0.45 mmol) in CH₂Cl₂ (6 mL) was added DMAP (18 mg, 0.15 mmol) and EDC.HCl (115 mg, 0.6 mmol). The reaction mixture was stirred at room temperature overnight, poured onto CH₂Cl₂ and saturated aqueous NaHCO₃ and extracted 2 times with CH₂Cl₂. Combined organic extract was dried over Na₂SO₄ and then concentrated in vacuo. Purification by chromatography twice (silica, 1.5-3% methanol in CH₂Cl₂ gradient) afforded 3-(4-fluorophenyl)-2-(4-methanesulfonylphenyl)-N-(5-{[(2-methoxy-ethyl)-methyl-amino]-methyl}-pyrazin-2-yl)-propionamide (40 mg, 18%): ¹H NMR (400 MHz, CHLOROFORM-d) δ ppm 2.31 (s, 3H) 2.64 (t, J=5.56 Hz, 2H) 2.99-3.12 (s, m, 4H) 3.32 (s, 3H) 3.50 (t, J=5.56 Hz, 2H) 3.57 (dd, J=13.64, 8.08 Hz, 1H) 3.72 (s, 2H) 3.85 (t, J=7.33 Hz, 1H) 6.86-6.99 (m, 2H) 7.08 (dd, J=8.59, 5.56 Hz, 2H) 7.55 (d, J=8.59 Hz, 2H) 7.83 (s, 1H) 7.91 (d, J=8.59 Hz, 2H) 8.29 (d, J=1.01 Hz, 1H) 9.41 (s, 1H), LC/MS-ESI observed [M+H]⁺ 501.

### Example 24

### N-{5-[(Benzyl-methyl-amino)-methyl]pyrazin-2-yl}-3-(4-fluorophenyl)-2-(4-methanesulfonyl-phenyl)-propionamide

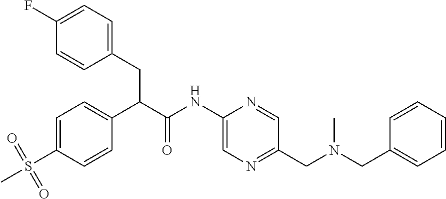

Preparation by a similar procedure to Example 23, except substituting benzyl-methyl-amine for (2-methoxyethyl)-methylamine in step 1, afforded N-{5-[(benzyl-methyl-amino)-methyl]pyrazin-2-yl}-3-(4-fluorophenyl)-2-(4-methanesulfonyl-phenyl)-propionamide (27 mg) as a foam: m.p. 99-100° C., LC/MS-ESI observed [M+H]⁺ 457.

### Example 25

### N-(6-Chloro-pyrazin-2-yl)-3-(4-fluoro-phenyl)-2-(4-methanesulfonyl-phenyl)-propionamide

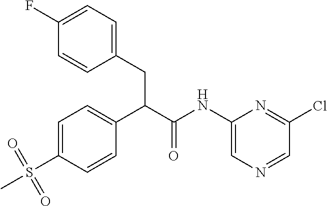

To a solution of 3-(4-fluorophenyl)-2-(4-methanesulfonylphenyl)propionic acid (262 mg, 0.8 mmol) and 6-chloro-pyrazin-2-ylamine (116 mg, 0.89 mmol) in CH₂Cl₂ was added DMAP (29 mg, 0.24 mmol) and EDC.HCl (186 mg, 0.97 mmol). The reaction mixture was stirred at room temperature for 3 days. Purification by chromatography twice (silica, 30-60% ethyl acetate/hexanes) afforded 3-(4-fluorophenyl)-2-(4-methanesulfonyl-phenyl)-N-(6-chloropyrazin-2-yl)-propionamide (196 mg, 57%): m.p. 191.6-192.7° C., LC/MS-ESI observed [M+H]⁺ 434.

### Example 26

### 3-(3-Fluoro-phenyl)-N-(5-hydroxymethylpyrazin-2-yl)-2-(4-methanesulfonylphenyl)-propionamide

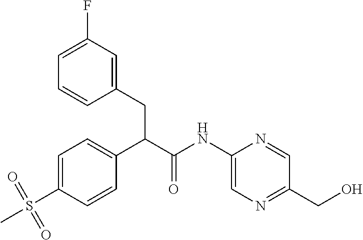

Step 1: To a solution of 2-methylpyrazine-5-carboxylic acid (10 g, 72.4 mmol) and TEA (20 mL, 108 mmol) in t-butanol (156 mL) and dioxanes (100 mL) was added diphenyl phosphorylazide (23.4 mL, 108 mmol) and the resulting solution was warmed at 100° C. for 3 hours and then cooled to room temperature overnight. The crude reaction mixture was concentrated in vacuo. Purification by chromatography (silica 0-15% ethyl acetate/hexanes) following trituration of impure fractions with ether afforded (5-methyl-pyrazin-2-yl)-carbamic acid tert-butyl ester (10.6 g, 70%): ¹H NMR (300 MHz, CHLOROFORM-d) δ ppm 1.56 (s, 9H) 2.51 (s, 3H) 8.02 (br. s., 1H) 8.07-8.14 (m, 1H) 9.18 (d, J=1.13 Hz, 1H).

Step 2: To a solution of (5-methyl-pyrazin-2-yl)-carbamic acid tert-butyl ester (from above and from example 21, step 1, 12.9 g, 61.6 mmol) and AIBN (10.1 g, 61 mmol) in CCl₄ (100 mL) was added NBS (11 g, 61.6 mmol) and the reaction mixture was stirred at 100° C. for 4 h. The reaction mixture was filtered, washed with CH₂Cl₂ and concentrated in vacuo. Purification by chromatography (silica, 4:5:1 CH₂Cl₂:hexanes:ethyl acetate) with repurification of mixed fractions by chromatography (silica, 4:5:1 CH₂Cl₂:hexanes:ethyl acetate) afforded a total of (5-bromomethyl-pyrazin-2-yl)-carbamic acid tert-butyl ester (13.6 g, 77%): ¹H NMR (300 MHz, CHLOROFORM-d) δ ppm 1.57 (s, 9H) 4.55 (s, 2H) 8.15 (s, 1H) 8.34 (d, J=1.51 Hz, 1H) 9.28 (d, J=1.51 Hz, 1H).

Step 3: To a suspension of potassium acetate (3.37 g, 34.2 mmol) and 18-crown-6 (0.72 g, 2.7 mmol) in dry acetonitrile (100 mL) under nitrogen was added (5-bromomethyl-pyrazin-2-yl)-carbamic acid tert-butyl ester (4.94 g, 17.1 mmol) in dry acetonitrile (100 mL). The reaction mixture was stirred at room temperature for 3 days and then concentrated in vacuo. The residue was taken up in CH₂Cl₂ and H₂O and extracted 3 times with CH₂Cl₂. Combined organic phase was dried over Na₂SO₄ and concentrated in vacuo. Purification by chromatography (silica, 6:4:2 hexanes:CH₂Cl₂:ethyl acetate) followed by trituration afforded acetic acid 5-tert-butoxycarbonylamino-pyrazin-2-ylmethyl ester (2.27 g, 50%): ¹H NMR (300 MHz, CHLOROFORM-d) δ ppm 1.57 (s, 9H) 2.13 (s, 3H) 5.20 (s, 2H) 8.28-8.34 (m, 2H) 9.31 (d, J=1.51 Hz, 1H).

Step 4: A solution of acetic acid 5-tert-butoxycarbonylamino-pyrazin-2-ylmethyl ester (423 mg, 1.58 mmol) in CH₂Cl₂ was added TFA (5 mL) and the resulting reaction solution was stirred at room temperature for 2 h. The reaction mixture was concentrated in vacuo, dissolved in CH₂Cl₂ and washed with H₂O. The organic phase was dried over Na₂SO₄ and concentrated in vacuo. Purification by chromatography (silica, 2% methanol in CH₂Cl₂) afforded acetic acid 5-amino-pyrazin-2-ylmethyl ester (147 mg, 56%): ¹H NMR (300 MHz, CHLOROFORM-d) δ ppm 2.11 (s, 3H) 4.64 (br. s., 1H) 5.09 (s, 2H) 7.99 (d, J=1.32 Hz, 1H) 8.08 (d, J=1.51 Hz, 1H).

Step 5: To a slurry of sodium hydride (0.42 g of 60% in oil dispersion, 10.5 mmol) in DMF (10 mL) under argon at 0° C. was added a solution of (4-methanesulfonylphenyl)-acetic acid methyl ester (2.0 g, 8.77 mmol) in DMF (10 mL). The reaction mixture was stirred at room temperature for 40 minutes. 3-fluorobenzyl bromide (1.13 mL, 9.2 mmol) was added. The reaction mixture was stirred at room temperature for 30 minutes and then quenched slowly with saturated aqueous NH₄Cl. The resultant mixture was poured onto 0.1% aqueous HCl and extracted 3 times with ether. The combined organic layer was washed with saturated aqueous NaCl, dried over Na₂SO₄ and concentrated in vacuo to afford a cream-colored solid upon sitting. Purification by triturated with 3:1 hexanes:ether afforded 3-(3-fluorophenyl)-2-(4-methanesulfonylphenyl)-propionic acid methyl ester (2.68 g, 91%): ¹H NMR (300 MHz, CHLOROFORM-d) δ ppm 2.94-3.09 (s, m, 4H) 3.44 (dd, J=13.75, 8.29 Hz, 1H) 3.65 (s, 3H) 3.90-3.99 (m, 1H) 6.74-6.96 (m, 3H) 7.20 (td, J=7.91, 6.03 Hz, 1H) 7.49 (d, J=8.29 Hz, 2H) 7.89 (d, J=8.48 Hz, 2H).

Step 6: A mixture of 3-(3-fluorophenyl)-2-(4-methanesulfonylphenyl)-propionic acid methyl ester (2.68 g, 8.0 mmol) in ethanol (75 mL) and 1 N aqueous KOH solution (20 mL) was stirred at room temperature for 3 days. The reaction mixture was poured onto 0.1% aqueous NaOH (250 mL) and ether (100 mL). The mixture was extracted with ether. Ether layer was washed with 0.01% aqueous NaOH. The combined aqueous phase was acidified with 10% aqueous HCl and extracted 3 times with CH₂Cl₂. The combined CH₂Cl₂ phase was washed with H2O, washed with saturated aqueous NaCl, dried over Na₂SO₄ and concentrated in vacuo to afford 3-(3-fluorophenyl)-2-(4-methane-sulfonylphenyl)-propionic acid (2.3 g, 90%) as a white solid: ¹H NMR (400 MHz, DMSO-d₆) δ ppm 3.05 (dd, J=14.15, 7.58 Hz, 1H) 3.20 (s, 3H) 3.33-3.39 (m, 1H) 4.12 (t, J=7.83 Hz, 1H) 6.99 (td, J=8.46, 2.27 Hz, 1H) 7.02-7.13 (m, 2H) 7.22-7.32 (m, 1H) 7.61 (d, J=8.59 Hz, 2H) 7.88 (d, J=8.59 Hz, 2H).

Step 7: To a solution of 3-(3-fluorophenyl)-2-(4-methanesulfonylphenyl)propionic acid (178 mg, 0.55 mmol) and acetic acid 5-amino-pyrazin-2-ylmethyl ester (92 mg, 0.55 mmol) in CH₂Cl₂ (10 mL) was added DMAP (20 mg, 0.16 mmol) and EDC.HCl (127 mg, 0.66 mmol). The reaction mixture was stirred at room temperature for 3 days. Purification by chromatography (silica, 30-50% ethyl acetate/hexanes in a stepwise gradient) afforded acetic acid 5-[3-(3-fluorophenyl)-2-(4-methanesulfonylphenyl)-propionylamino]-pyrazin-2-ylmethyl ester (125 mg, 48%): ¹H NMR (300 MHz, CHLOROFORM-d) δ ppm 2.12 (s, 3H) 2.99-3.14 (s, m, 4H) 3.60 (dd, J=13.75, 8.10 Hz, 1H) 3.92 (t, J=7.54 Hz, 1H) 5.19 (s, 2H) 6.79-6.95 (m, 3H) 7.20 (td, J=7.91, 6.03 Hz, 1H) 7.55 (d, J=8.48 Hz, 2H) 7.85-7.98 (m, 3H) 8.24 (d, J=1.51 Hz, 1H) 9.48 (d, J=1.32 Hz, 1H) LC/MS-ESI observed [M+H]⁺ 472.

Step 8: To a solution of acetic acid 5-[3-(3-fluorophenyl)-2-(4-methanesulfonylphenyl)-propionyl-amino]-pyrazin-2-ylmethyl ester (119 mg, 0.25 mmol) in methanol (5 mL) was added K₂CO₃ (40 mg, 0.3 mmol) and the resultant mixture was stirred at room temperature for 30 minutes. The reaction mixture was poured H₂O and 0.1% aqueous HCl and extracted 3 times with CH₂Cl₂. The combined organic extract was washed with saturated aqueous NaCl, dried over NaSO₄ and concentrated in vacuo to afford 3-(3-fluorophenyl)-N-(5-hydroxymethylpyrazin-2-yl)-2-(4-methanesulfonylphenyl)-propionamide (106 mg, 99%): m.p. 82.8-98.7° C., LC/MS-ESI observed [M+H]⁺ 430.

### Example 27

### N-Benzooxazol-2-yl-3-(4-fluorophenyl)-2-(4-methanesulfonyl-2-methyl-phenyl)-propionamide

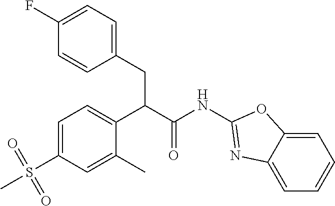

Step 1: To a solution of 4-bromo-3-methylthioanisole (280 mg, 1.3 mmol) under argon in THF (6 mL) in a microwave vial was added a solution of the 2-tert-butoxy-2-oxoethylzinc chloride (Rieke Product List, 6 mL of 0.5 M in ether, 3 mmol), tetrakis(triphenylphospine)-Palladium(0) (600 mg, 0.52 mmol) and the mixture was heated at 120° C. for 5 minutes in the microwave. The crude reaction mixture was poured onto H₂O and extracted 5 times with CH₂Cl₂. Combined organic extract was dried over Na₂SO₄ and concentrated in vacuo. Purification by chromatography (silica, 5-10% ethyl acetate/hexanes in a gradient) afforded (2-methyl-4-methylsulfanylphenyl)-acetic acid tert-butyl ester (157 mg, 48%): ¹H NMR (300 MHz, CHLOROFORM-d) δ ppm 1.43 (s, 9H) 2.28 (s, 3H) 2.46 (s, 3H) 3.49 (s, 2H) 7.00-7.14 (m, 3 H).

Step 2: To a slurry of sodium hydride (30 mg of 60% in oil dispersion, 0.74 mmol) in DMF under argon at 0° C. was added a solution of (2-methyl-4-methylsulfanylphenyl)-acetic acid tert-butyl ester (157 mg, 0.62 mmol) in DMF (3 mL). The reaction mixture was stirred at room temperature for 40 minutes. 4-fluorobenzyl bromide (0.08 mL, 0.65 mmol) was added. The reaction mixture was stirred at room temperature for 2 h and then quenched slowly with saturated aqueous NH₄Cl. The resultant mixture was poured onto 0.1% aqueous HCl and extracted 3 times with ether. The combined organic layer was washed with saturated aqueous NaCl, dried over Na₂SO₄ and concentrated in vacuo. Purification by chromatography (silica, 5% ethyl acetate/hexanes) afforded 3-(4-fluorophenyl)-2-(2-methyl-4-methylsulfanyl-phenyl)-propionic acid tert-butyl ester (155 mg, 69%).

Step 3: To a solution of 3-(4-fluorophenyl)-2-(2-methyl-4-methylsulfanyl-phenyl)-propionic acid tert-butyl ester (155 mg, 0.43 mmol) in CH₂Cl₂ (10 mL) was added m-CPBA (118 mg, 0.52 mmol) and the reaction mixture was stirred at room temperature for 1 h. Additional m-CPBA (118 mg, 0.52 mmol) was added and the reaction mixture was stirred overnight. The reaction mixture was poured onto aqueous NaHCO₃ and extracted 3 times with CH₂Cl₂. Combined organic extract was dried over Na₂SO₄ and concentrated in vacuo. The residue was dissolved in CH₂Cl₂ (10 mL) and to that was added TFA (1.5 mL). The mixture was stirred at room temperature for 3 h, concentrated in vacuo, mixed with CH₂Cl₂ and 1% aqueous NaOH and extracted with CH₂Cl₂. CH₂Cl₂ layer was discarded. The aqueous phase was acidified with 10% aqueous HCl and was extracted 3 times with CH₂Cl₂. This organic phase was dried over Na₂SO₄ and concentrated in vacuo to afford 3-(4-fluorophenyl)-2-(4-methanesulfonyl-2-methyl-phenyl)-propionic acid (131 mg, 90%):

Step 4: To a solution of 3-(4-fluoro-phenyl)-2-(4-methanesulfonyl-2-methyl-phenyl)-propionic acid (131 mg, 0.39 mmol) and 2-aminobenzoxazole (67 mg, 0.5 mmol) in CH₂Cl₂ (6 mL) was added DMAP (15 mg, 0.12 mmol) and EDC.HCl (96 mg, 0.5 mmol). The reaction mixture was stirred at room temperature overnight, poured onto 0.1% aqueous HCl and extracted 3 times with CH₂Cl₂. The combined organic extract was dried over Na₂SO₄ and concentrated in vacuo. Purification by chromatography (silica, 1% methanol in CH₂Cl₂) followed by re-purification by chromatography (silica, 50% ethyl acetate/hexanes) afforded N-benzooxazol-2-yl-3-(4-fluorophenyl)-2-(4-methane-sulfonyl-2-methyl-phenyl)-propionamide (60 mg, 34%): ¹H NMR (400 MHz, DMSO-d₆) δ ppm 2.42 (s, 3H) 2.95 (dd, J=13.64, 6.06 Hz, 1H) 3.18 (s, 3H) 3.38 (m, 2H) 7.09 (t, J=8.84 Hz, 2H) 7.20-7.34 (m, 4H) 7.57 (dd, J=18.69, 7.07 Hz, 2H) 7.67-7.83 (m, 3H) 11.91 (s, 1H).

### Example 28

### N-Benzooxazol-2-yl-2,3-bis-(4-fluoro-phenyl)-propionamide

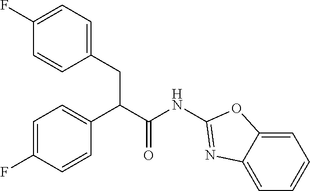

Step 1: To a slurry of sodium hydride (0.29 g of 60% in oil dispersion, 7.3 mmol) in DMF (10 mL) under argon at 0° C. was added a solution of (4-fluorophenyl)-acetic acid methyl ester (1.03 g, 6.13 mmol) in DMF (10 mL). The reaction mixture was stirred at room temperature for 30 minutes. 4-fluorobenzyl bromide (1.13 mL, 9.2 mmol) was added. The reaction mixture was stirred at room temperature for 20 minutes and then quenched slowly with saturated aqueous NH₄Cl. The resultant mixture was poured onto 0.1% aqueous HCl and extracted 3 times with ether. The combined organic layer was washed with saturated NaCl, dried over Na₂SO₄ and concentrated in vacuo. Purification by chromatography (silica, 5% ethyl acetate/hexanes) afforded 2,3-bis-(4-fluoro-phenyl)-propionic acid methyl ester (1.15 g). A solution of the 2,3-bis-(4-fluoro-phenyl)-propionic acid methyl ester (1.15 g) in ethanol (100 mL) and 1 N aqueous KOH solution (12 mL) was stirred at room temperature for 4 h. The reaction mixture was concentrated to 50 mL, poured onto 0.1% aqueous NaOH and ether. The mixture was extracted with ether. Ether layer was washed with 0.01% aqueous NaOH. The combined aqueous phase was acidified with 1 N aqueous HCl and extracted 3 times with CH₂Cl₂. The combined CH₂Cl₂ phase was washed with H₂O, washed with saturated NaCl, dried over Na₂SO₄ and concentrated in vacuo to afford 2,3-bis-(4-fluoro-phenyl)-propionic acid (0.74 g, 46%): ¹H NMR (300 MHz, CHLOROFORM-d) δ ppm 2.97 (dd, J=13.75, 7.54 Hz, 1H) 3.34 (dd, J=13.94, 7.91 Hz, 1H) 3.78 (t, J=7.82 Hz, 1H) 6.84-7.07 (m, 6H) 7.18-7.28 (m, 2H).

Step 2: To a solution of 2,3-bis-(4-fluoro-phenyl)-propionic acid (411 mg, 1.57 mmol) and 2-aminobenzoxazole (221 mg, 1.65 mmol) in CH₂Cl₂ (75 mL) was added DMAP (61 mg, 0.5 mmol) and EDC.HCl (380 mg, 2 mmol). The reaction mixture was stirred at room temperature 7 days, poured onto 0.1% aqueous HCl and extracted 3 times with CH₂Cl₂. The combined organic extract was dried over Na₂SO₄ and concentrated in vacuo. Purification by chromatography (silica, 10-15% ethyl acetate/hexanes stepwise gradient) followed by trituration with 5% CH₂Cl₂ in ether afforded N-benzooxazol-2-yl-2,3-bis-(4-fluoro-phenyl)-propionamide (289 mg, 49%): LC/MS-ESI observed [M+H]⁺ 379.

### Example 29

### N-Benzooxazol-2-yl-2-(2,4-difluoro-phenyl)-3-(4-fluoro-phenyl)-propionamide

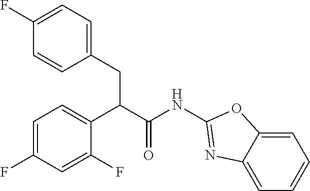

Preparation by a similar procedure to Example 28, except substituting (2,4-difluoro-phenyl)-acetic acid methyl ester for (4-fluorophenyl)-acetic acid methyl ester in step 1, afforded N-benzooxazol-2-yl-2-(2,4-difluoro-phenyl)-3-(4-fluoro-phenyl)-propionamide (93 mg, 28%): m.p. 177.5-177.9° C., LC/MS-ESI observed [M+H]⁺ 397.

### Example 30

### 4-[1-(Benzooxazol-2-ylcarbamoyl)-2-(4-fluorophenyl)-ethyl]-benzoic acid ethyl ester

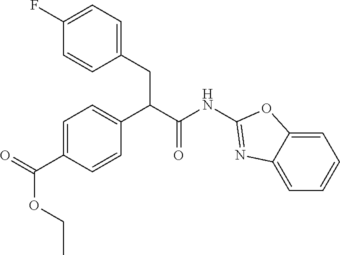

Step 1: To a solution of ethyl bromoacetate (1.34 g, 5.85 mmol) under argon in THF (24 mL), divided into 3 microwave vials was added a solution of the 2-tert-Butoxy-2-oxoethylzinc chloride (Rieke Product List, 7 mL of 0.5 M in ether per microwave vial, 10.5 mmol total), tetrakis(triphenylphospine)-Palladium(0) (400 mg per microwave vial, 1.03 mmol total) and the tubes were sealed and heated at 120° C. for 6 minutes in the microwave. The crude reaction mixtures were combined, quenched with saturated aqueous NH₄Cl, poured onto H₂O and extracted 5 times with 1% methanol in CH₂Cl₂. Combined organic extract was washed with saturated NaCl, dried over NaSO₄ and concentrated in vacuo. Purification by chromatography twice (silica, 5% ethyl acetate/hexanes in a gradient) afforded 4-tert-butoxycarbonylmethyl-benzoic acid ethyl ester (1.08 g, 70%): ¹H NMR (300 MHz, CHLOROFORM-d) δ ppm 1.39 (t, J=7.16 Hz, 3H) 1.43 (s, 9H) 3.58 (s, 2H) 4.37 (q, J=7.03 Hz, 2H) 7.34 (dd, J=8.10, 0.57 Hz, 2H) 8.00 (d, J=8.48 Hz, 2H).

Step 2: To a slurry of sodium hydride (196 mg of 60% in oil dispersion, 4.91 mmol) in DMF (8 mL) under argon at 0° C. was added a solution of 4-tert-butoxycarbonylmethyl-benzoic acid ethyl ester (1.08 g, 4.09 mmol) in DMF (8 mL). The reaction mixture was stirred at room temperature for 40 minutes. 4-fluorobenzyl bromide (0.526 mL, 4.29 mmol) was added. The reaction mixture was stirred at room temperature for 1 h and then quenched slowly with saturated aqueous (NH₄)₂SO₄. The resultant mixture was poured onto 0.1% aqueous HCl and extracted 3 times with ether. The combined organic layer was washed with saturated aqueous NaCl, dried over Na₂SO₄ and concentrated in vacuo. Purification by chromatography (silica, 5% ethyl acetate/hexanes) afforded 4-[1-tert-butoxycarbonyl-2-(4-fluoro-phenyl)-ethyl]-benzoic acid ethyl ester (0.92 g, 60%) as a white solid: ¹H NMR (300 MHz, CHLOROFORM-d) δ ppm 1.32 (s, 9H) 1.39 (t, J=7.16 Hz, 3H) 2.95 (dd, J=13.75, 7.16 Hz, 1H) 3.26-3.40 (m, 1H) 3.75 (dd, J=8.57, 7.06 Hz, 1H) 4.37 (q, J=7.03 Hz, 2H) 6.81-6.98 (m, 2H) 7.06 (dd, J=8.76, 5.37 Hz, 2H) 7.34 (d, J=8.29 Hz, 2H) 7.96 (s, 2H).

### Step 3: To a solution of 4-[1-tert-butoxycarbonyl-2-(4-fluoro-phenyl)-ethyl]-benzoic acid ethyl ester (0.92 g 2.47 mmol) in CH₂Cl₂ (20 mL) was added TFA (7 mL). The mixture was stirred at room temperature for 5 h, concentrated in vacuo, mixed with CH₂Cl₂ and 5% aqueous NaHCO₃ and extracted into ether. Ether layer was backwashed with 5% aqueous NaHCO₃ and discarded. The aqueous phase was acidified with 10% aqueous HCl and was extracted 3 times with CH₂Cl₂. This organic phase was dried over Na₂SO₄ and concentrated in vacuo to afford 4-[1-carboxy-2-(4-fluoro-phenyl)-ethyl]-benzoic acid ethyl ester (650 mg, 83%).

Step 4: To a solution of 4-[1-carboxy-2-(4-fluoro-phenyl)-ethyl]-benzoic acid ethyl ester (650 mg, 2.05 mmol) and 2-aminobenzoxazole (290 mg, 2.16 mmol) in CH₂Cl₂ (25 mL) was added DMAP (75 mg, 0.615 mmol) and EDC.HCl (472 mg, 2.46 mmol). The reaction mixture was stirred at room temperature overnight and then concentrated in vacuo. Purification by chromatography (silica, 20% ethyl acetate/hexanes) afforded 4-[1-(benzooxazol-2-ylcarbamoyl)-2-(4-fluorophenyl)-ethyl]-benzoic acid ethyl ester (722 mg, 82%): m.p. 122-125° C., LC/MS-ESI observed [M+H]⁺ 433.

### Example 31

### N-Benzooxazol-2-yl-3-(4-fluorophenyl)-2-[4-(1-hydroxy-1-methyl-ethyl)-phenyl]-propionamide

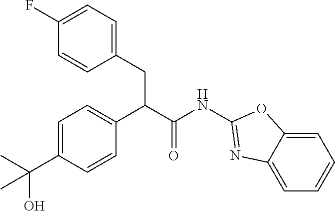

To a solution of 4-[1-(benzooxazol-2-ylcarbamoyl)-2-(4-fluorophenyl)-ethyl]-benzoic acid ethyl ester (112 mg, 0.26 mmol) in THF (3 mL) at 0° C. under argon was added methylmagnesium bromide (0.6 mL of 1 M, 0.6 mmol). The reaction was warmed to room temperature over 30 minutes and stirred at room temperature for 90 minutes. Additional methylmagnesium bromide (0.5 mL of 1 M, 0.5 mmol) was added and the reaction mixture was stirred at room temperature for 1 h. Additional methylmagnesium bromide (0.5 mL of 1 M, 0.5 mmol) was added and the reaction mixture was stirred at room temperature for 30 minutes. A solution of saturated citric acid was added, the mixture was poured onto 1% aqueous HCl and was extracted 3 times with CH₂Cl₂. Combined organic layer was dried over Na₂SO₄ and concentrated in vacuo. Purification by chromatography (silica, 40% ethyl acetate/hexanes) afforded N-benzooxazol-2-yl-3-(4-fluorophenyl)-2-[4-(1-hydroxy-1-methyl-ethyl)-phenyl]-propionamide (90 mg, 83%):

m.p. 87-90° C., LC/MS-ESI observed [M+H]⁺ 419.

### Example 32

### 4-[1-(Benzooxazol-2-ylcarbamoyl)-2-(4-fluoro-phenyl)-ethyl]-benzamide

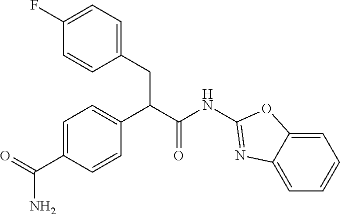

Step 1: A mixture of 4-[1-(benzooxazol-2-ylcarbamoyl)-2-(4-fluorophenyl)-ethyl]-benzoic acid ethyl ester (0.459 g, 1.06 mmol) in ethanol (30 mL) and 1 N aqueous KOH solution (3 mL) was stirred at room temperature 1 h. Additional 1 N aqueous KOH solution (3 mL) was added and reaction mixture was stirred and additional 1 h. Additional 1 N aqueous KOH solution (3 mL) was added and reaction mixture was stirred and additional 2 h. The reaction mixture concentrated to 20% volume. The mixture was poured onto H₂O and washed 2 times with CH₂Cl₂. The aqueous phase was acidified in aqueous HCl and 1% methanol in CH₂Cl₂ was added. The mixture was filtered and the solid washed 2 times with water and then 3 times with ether to afford 4-[1-(benzooxazol-2-ylcarbamoyl)-2-(4-fluoro-phenyl)-ethyl]-benzoic acid (90 mg). The filtrate was extracted 3 times with 5% methanol in CH₂Cl₂. The organic phase was dried over Na₂SO₄ and concentrated in vacuo to afford additional 4-[1-(benzooxazol-2-ylcarbamoyl)-2-(4-fluoro-phenyl)-ethyl]-benzoic acid (300 mg, 91% total yield): m.p. 199-201° C., LC/MS-ESI observed [M−H]⁺ 403.

Step 2: To a solution of 4-[1-(benzooxazol-2-ylcarbamoyl)-2-(4-fluoro-phenyl)-ethyl]-benzoic acid (85 mg, 0.21 mmol) in DMF was added HOAt (34 mg, 0.25 mmol), EDC.HCl (60 mg, 0.31 mmol) and DIEA (100 μL, 0.57 mmol). To the resultant solution was added NH₄Cl (22 mg, 0.42 mmol) and the mixture was stirred at room temperature overnight and subsequently concentrated in vacuo. The residue was taken up as a slurry in CH₂Cl₂ and poured onto 0.1% aqueous HCl. The mixture remained a slurry. Methanol (5-10 drops) was added, but mixture remained a slurry. The slurry was diluted with an equal volume of ether and filtered. The collected solid was washed 5 times with H₂O, washed 2 times with ether and dried under vacuum to afford 4-[1′-(benzooxazol-2-ylcarbamoyl)-2-(4-fluoro-phenyl)-ethyl]-benzamide (57 mg, 67%) as a white solid: m.p. 137-139° C., LC/MS-ESI observed [M+H]⁺ 404.

### Example 33

### 4-[1-(Benzooxazol-2-ylcarbamoyl)-2-(4-fluorophenyl)-ethyl]-N-(2-hydroxethyl)-benzamide

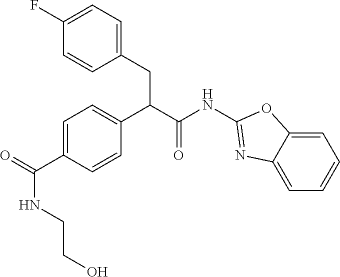

To a solution of 4-[1-(benzooxazol-2-ylcarbamoyl)-2-(4-fluoro-phenyl)-ethyl]-benzoic acid (85 mg, 0.21 mmol) in DMF was added HOAt (34 mg, 0.25 mmol), EDC.HCl (60 mg, 0.31 mmol) and DIEA (100 μL, 0.57 mmol). To the resultant solution was added 2-hyroxyethylamine (15 μL, 0.25 mmol) and the mixture was stirred at room temperature overnight and subsequently concentrated in vacuo. The residue was taken up in 5% methanol in CH₂Cl₂, poured onto 0.1% aqueous HCl and extracted 3 times with CH₂Cl₂. The combined organic extract was dried over Na₂SO₄ and concentrated in vacuo. Purification by chromatography twice (silica, 4-5% methanol in CH₂Cl₂) afforded 4-[1-(benzooxazol-2-ylcarbamoyl)-2-(4-fluorophenyl)-ethyl]-N-(2-hydroxethyl)-benzamide (68 mg, 72%): m.p. 134-136° C., LC/MS-ESI observed [M+H]⁺ 448.

### Example 34

### 4-[1-(Benzooxazol-2-ylcarbamoyl)-2-(4-fluorophenyl)-ethyl]-N-(2-dimethylamino-ethyl)-benzamide

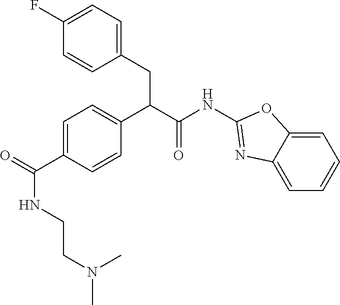

To a solution of 4-[1-(benzooxazol-2-ylcarbamoyl)-2-(4-fluoro-phenyl)-ethyl]-benzoic acid (85 mg, 0.21 mmol) in DMF was added HOAt (34 mg, 0.25 mmol), EDC.HCl (60 mg, 0.31 mmol) and DIEA (100 μL, 0.57 mmol). To the resultant solution was added N¹,N¹-Dimethylethane-1,2-diamine (28 μL, 0.25 mmol) and the mixture was stirred at room temperature overnight and subsequently concentrated in vacuo. Purification by chromatography [silica, 30-40% (60:10:1 CH₂Cl₂:methanol:NH₄OH)/CH₂Cl₂ gradient) followed by trituration with ether afforded 4-[1-(benzooxazol-2-ylcarbamoyl)-2-(4-fluorophenyl)-ethyl]-N-(2-dimethylaminoethyl)-benzamide (60 mg, 60%): m.p. 149-151° C., LC/MS-ESI observed [M+H]⁺ 475.

### Example 35

### 3-(4-Fluoro-phenyl)-2-(4-methanesulfonyl-phenyl)-N-(4-methyl-benzooxazol-2-yl)-propionamide

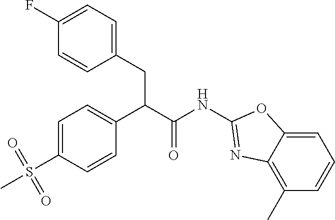

A mixture of 3-(4-fluorophenyl)-2-(4-methanesulfonylphenyl)propionic acid (prepared in example 14, 175 mg, 0.54 mmol), 4-methyl-benzooxazol-2-ylamine (119 mg, 0.8 mmol), DMAP (20 mg, 0.16 mmol) and EDC.HCl (136 mg, 0.71 mmol) in CH₂Cl₂ (10 mL) was stirred at room temperature overnight and then concentrated in vacuo. Purification by chromatography (silica, 50% ethyl acetate/hexanes) afforded 3-(4-fluorophenyl)-2-(4-methanesulfonyl-phenyl)-N-(4-methyl-benzooxazol-2-yl)-propionamide (237 mg, 97%): LC/MS-ESI observed [M+H]⁺ 453.

### Example 36

### 3-(4-Fluoro-phenyl)-2-(4-methanesulfonyl-phenyl)-N-(6-methyl-benzooxazol-2-yl)-propionamide

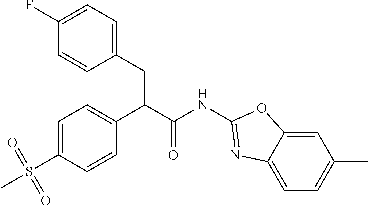

Preparation by a similar procedure to Example 35, except substituting 6-methyl-benzooxazol-2-ylamine for 4-methyl-benzooxazol-2-ylamine afforded 3-(4-fluoro-phenyl)-2-(4-methanesulfonyl-phenyl)-N-(6-methyl-benzooxazol-2-yl)-propionamide (226 mg, 92%): LC/MS-ESI observed [M+H]⁺ 453.

### Example 37

### 3-(4-Fluoro-phenyl)-2-(4-methanesulfonyl-phenyl)-N-(5-methyl-benzooxazol-2-yl)-propionamide

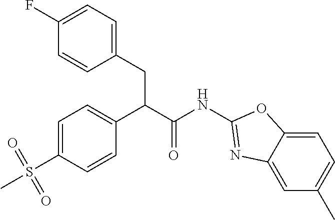

Preparation by a similar procedure to Example 35, except substituting 5-methyl-benzooxazol-2-ylamine for 4-methyl-benzooxazol-2-ylamine afforded 3-(4-fluoro-phenyl)-2-(4-methanesulfonyl-phenyl)-N-(5-methyl-benzooxazol-2-yl)-propionamide (180 mg, 73%): LC/MS-ESI observed [M+H]⁺ 453.

### Example 38

### 3-(4-Fluoro-phenyl)-2-(4-methanesulfonyl-phenyl)-N-(6-chloro-benzooxazol-2-yl)-propionamide

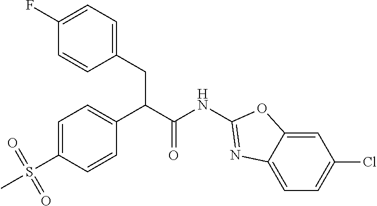

Preparation by a similar procedure to Example 35, except substituting 6-chloro-benzooxazol-2-ylamine for 4-methyl-benzooxazol-2-ylamine afforded 3-(4-fluoro-phenyl)-2-(4-methanesulfonyl-phenyl)-N-(6-chloro-benzooxazol-2-yl)-propionamide (227 mg, 89%): LC/MS-ESI observed [M+H]⁺ 473.

### Example 39

### 3-(4-Fluoro-phenyl)-2-(4-methanesulfonyl-phenyl)-N-(5-chloro-benzooxazol-2-yl)-propionamide

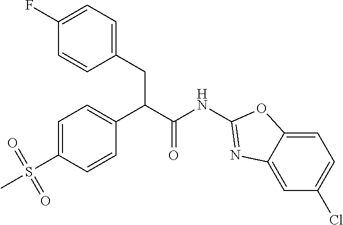

Preparation by a similar procedure to Example 35, except substituting 5-chloro-benzooxazol-2-ylamine for 4-methyl-benzooxazol-2-ylamine afforded 3-(4-fluoro-phenyl)-2-(4-methanesulfonyl-phenyl)-N-(5-chloro-benzooxazol-2-yl)-propionamide (131 mg, 51%): LC/MS-ESI observed [M+H]⁺ 473.

### Example 40

### 3-(4-Fluoro-phenyl)-2-(4-methanesulfonyl-phenyl)-N-(6-fluoro-benzooxazol-2-yl)-propionamide

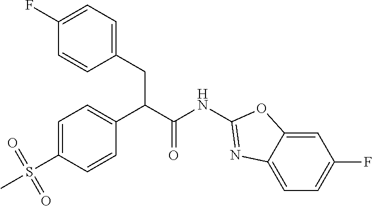

Step 1: To a solution of 2-amino-5-fluorophenol (127 mg, 1 mmol) in anhydrous THF (5 mL) was added (di-imidazol-1-yl)-methyleneamine (prepared in example 20, 161 mg, 1 mmol) and the resulting solution was heated at 60° C. for 3 days. The reaction was diluted with ethyl acetate (20 mL) and washed with H₂O, saturated aqueous NH₄Cl and saturated aqueous NaCl and dried over Na₂SO₄. The mixture was concentrated in vacuo. Purification by chromatography (silica, 50% ethyl acetate/hexanes) afforded 6-fluorobenzooxazol-2-ylamine (87 mg, 57%): ¹H NMR (300 MHz, MeOH) δ ppm 3.29 (s, 2H) 6.01 (ddd, J=9.89, 8.57, 2.45 Hz, 1H) 6.12 (dd, J=8.01, 2.54 Hz, 1H) 6.29 (dd, J=8.57, 4.80 Hz, 1H).

Step 2: A mixture of 3-(4-fluorophenyl)-2-(4-methanesulfonylphenyl)propionic acid (prepared in example 14, 175 mg, 0.54 mmol), 6-fluorobenzooxazol-2-ylamine (87 mg, 0.57 mmol), DMAP (20 mg, 0.16 mmol) and EDC.HCl (125 mg, 0.65 mmol) in CH₂Cl₂ (10 mL) was stirred at room temperature overnight and then concentrated in vacuo. Purification by chromatography (silica, 50% ethyl acetate/hexanes) afforded 3-(4-fluorophenyl)-2-(4-methanesulfonyl-phenyl)-N-(6-fluoro-benzooxazol-2-yl)-propionamide (206 mg, 79%): LC/MS-ESI observed [M+H]⁺ 457.

### Example 41

### 3-(4-Fluoro-phenyl)-2-(4-methanesulfonyl-phenyl)-N-(5-trifluoromethyl-benzooxazol-2-yl)-propionamide

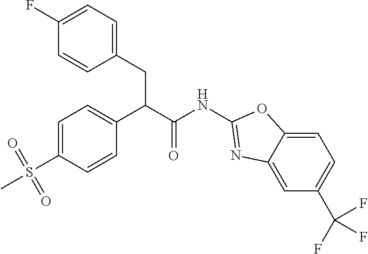

Preparation by a similar procedure to Example 40, except substituting 2-amino-4-trifluoromethylphenol for 2-amino-5-fluorophenol afforded 3-(4-fluoro-phenyl)-2-(4-methanesulfonyl-phenyl)-N-(5-trifluoromethyl-benzooxazol-2-yl)-propionamide (116 mg, 23%): LC/MS-ESI observed [M+H]⁺ 507.

### Example 42

### 3-(4-Fluoro-phenyl)-2-(4-methanesulfonyl-phenyl)-N-(7-methyl-benzooxazol-2-yl)-propionamide

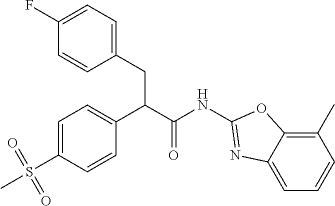

Preparation by a similar procedure to Example 40, except substituting 2-amino-6-methylphenol for 2-amino-5-fluorophenol afforded 3-(4-fluoro-phenyl)-2-(4-methanesulfonyl-phenyl)-N-(7-methyl-benzooxazol-2-yl)-propionamide (144 mg, 32%): LC/MS-ESI observed [M+H]⁺ 453.

### Example 43

### N-(5,7-Dimethyl-benzooxazol-2-yl)-3-(4-fluoro-phenyl)-2-(4-methanesulfonyl-phenyl)-propionamide

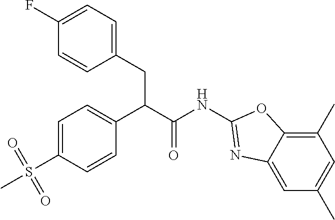

Preparation by a similar procedure to Example 40, except substituting 2-amino-4,6-dimethylphenol for 2-amino-5-fluorophenol afforded N-(5,7-dimethyl-benzooxazol-2-yl)-3-(4-fluoro-phenyl)-2-(4-methanesulfonyl-phenyl)-propionamide (53.8 mg, 11%): LC/MS-ESI observed [M+H]⁺ 467.

### Example 44

### 3-(4-Fluoro-phenyl)-2-(4-methanesulfonyl-phenyl)-N-(5-methoxy-benzooxazol-2-yl)-propionamide

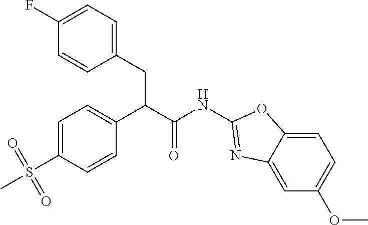

Preparation by a similar procedure to Example 40, except substituting 2-amino-4-methoxylphenol for 2-amino-5-fluorophenol afforded 3-(4-fluoro-phenyl)-2-(4-methanesulfonyl-phenyl)-N-(5-methoxy-benzooxazol-2-yl)-propionamide (105 mg, 22%): LC/MS-ESI observed [M+H]⁺ 469.

### Example 45

### 2-[3-(4-Fluoro-phenyl)-2-(4-methanesulfonyl-phenyl)-propionylamino]-benzooxazole-6-carboxylic acid methyl ester

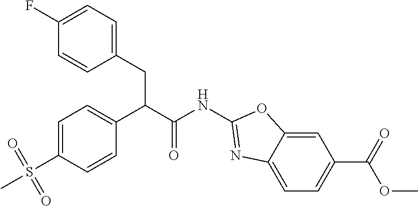

Preparation by a similar procedure to Example 40, except substituting 4-amino-3-hydroxy-benzoic acid methyl ester for 2-amino-5-fluorophenol afforded 2-[3-(4-fluoro-phenyl)-2-(4-methanesulfonyl-phenyl)-propionylamino]-benzooxazole-6-carboxylic acid methyl ester (110 mg, 22%): LC/MS-ESI observed [M+H]⁺ 497.

### Example 46

### N-(5-Fluoro-benzooxazol-2-yl)-3-(4-fluoro-phenyl)-2-(4-methanesulfonyl-phenyl)-propionamide

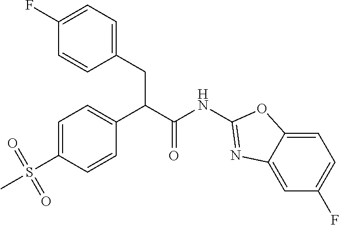

Preparation by a similar procedure to Example 40, except substituting 2-amino-4-fluorphenol for 2-amino-5-fluorophenol afforded N-(5-fluoro-benzooxazol-2-yl)-3-(4-fluoro-phenyl)-2-(4-methanesulfonyl-phenyl)-propionamide (140 mg, 31%): LC/MS-ESI observed [M+H]⁺ 457.

### Example 47

### 3-(4-Fluoro-phenyl)-2-(4-methanesulfonyl-phenyl)-N-[5-(pyrrolidine-1-sulfonyl)-benzooxazol-2-yl]-propionamide

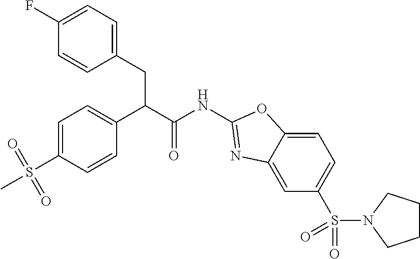

Preparation by a similar procedure to Example 35, except substituting 5-(pyrrolidine-1-sulfonyl)-benzooxazol-2-ylamine for 4-methyl-benzooxazol-2-ylamine afforded 3-(4-fluoro-phenyl)-2-(4-methanesulfonyl-phenyl)-N-[5-(pyrrolidine-1-sulfonyl)-benzooxazol-2-yl]-propionamide (165 mg, 54%): LC/MS-ESI observed [M−H]⁺ 570.

### Example 48

### 3-(4-Fluoro-phenyl)-2-(4-methanesulfonyl-phenyl)-N-(5-phenyl-oxazol-2-yl)-propionamide

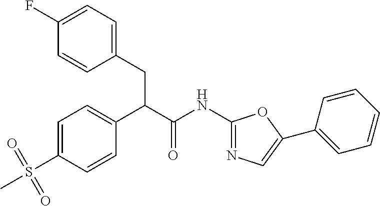

Step 1: To a solution of 2-hydroxyacetophenone (1.0 g, 7.4 mmol) in ethanol (20 mL) was added cyanamide (400 mg, 9.4 mmol) and the reaction mixture was heated at reflux under nitrogen overnight. The reaction mixture was poured into aqueous 6N HCl (40 mL) and washed with ethyl acetate and ethyl acetate layer discarded. The aqueous layer was made alkaline with aqueous NaOH and extracted 3 times with ethyl acetate. The combined organic phase was dried over Na₂SO₄ and concentrated in vacuo to afford 5-phenyl-oxazol-2-ylamine (250 mg, 21%):

Step 2: Preparation by a similar procedure to Example 35, except substituting 5-phenyl-oxazol-2-ylamine for 4-methyl-benzooxazol-2-ylamine afforded 3-(4-fluoro-phenyl)-2-(4-methanesulfonyl-phenyl)-N-(5-phenyl-oxazol-2-yl)-propionamide (107 mg, 43%): ¹H NMR (300 MHz, DMSO-d₆) δ ppm 3.06 (dd, J=13.47, 6.69 Hz, 1H) 3.19 (s, 3H) 3.44 (dd, J=13.66, 8.95 Hz, 1H) 4.05 (d, J=7.16 Hz, 2H) 7.02-7.15 (m, 2H) 7.17-7.49 (m, 6H) 7.52 (s, 1H) 7.55-7.63 (m, 2H) 7.68 (d, J=8.48 Hz, 2H) 7.92 (d, J=8.29 Hz, 2H).

### Example 49

### Acetic acid 5-[3-(4-fluoro-phenyl)-2-(4-methanesulfonyl-phenyl)-propionylamino]-pyrazin-2-ylmethyl ester

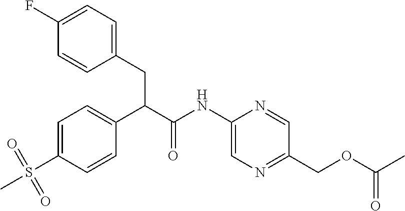

To a solution of acetic acid 5-tert-butoxycarbonylamino-pyrazin-2-ylmethyl ester (prepared in example 26, 500 mg, 1.87 mmol) in CH₂Cl₂ (20 mL) was added TFA (5 mL) and the resulting reaction solution was stirred at room temperature for 3.5 h. The reaction mixture was concentrated in vacuo to afford acetic acid 5-amino-pyrazin-2-ylmethyl ester trifluoroacetate (528 mg, 100%): ¹H NMR (300 MHz, CHLOROFORM-d) δ ppm 2.15 (s, 3H) 5.15 (s, 2H) 7.82 (d, J=0.57 Hz, 1H) 8.48 (d, J=1.32 Hz, 1H).

Step 2: A mixture of 3-(4-fluorophenyl)-2-(4-methanesulfonylphenyl)propionic acid (prepared in example 14, 504 mg, 1.56 mmol), acetic acid 5-amino-pyrazin-2-ylmethyl ester trifluoroacetate (528 mg, 1.87 mmol), DMAP (57 mg, 0.47 mmol) and EDC.HCl (360 mg, 1.87 mmol) in CH₂Cl₂ (30 mL) was stirred at room temperature overnight and then concentrated in vacuo. Purification by chromatography (silica, 50% ethyl acetate/hexanes) afforded acetic acid 5-[3-(4-fluoro-phenyl)-2-(4-methanesulfonyl-phenyl)-propionylamino]-pyrazin-2-ylmethyl ester (530 mg, 72%): ¹H NMR (400 MHz, CHLOROFORM-d) δ ppm 2.12 (s, 3H) 2.97-3.13 (s, m, 4H) 3.57 (dd, J=13.89, 8.34 Hz, 1H) 3.88 (t, J=7.58 Hz, 1H) 5.19 (s, 2H) 6.92 (t, J=8.59 Hz, 2H) 7.08 (dd, J=8.59, 5.56 Hz, 2H) 7.55 (d, J=8.08 Hz, 2H) 7.90 (s, d, J=8.08 Hz, 3H) 8.23 (d, J=1.52 Hz, 1H) 9.48 (s, 1H).

### Example 50

### 3-(4-Fluoro-phenyl)-N-(5-hydroxymethyl-pyrazin-2-yl)-2-(4-methanesulfonyl-phenyl)-propionamide

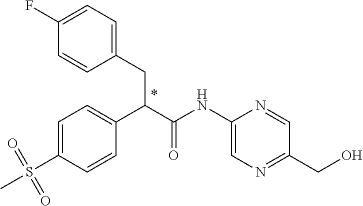

To a solution of acetic acid 5-[3-(4-fluorophenyl)-2-(4-methanesulfonylphenyl)-propionyl-amino]-pyrazin-2-ylmethyl ester (485 mg, 1.03 mmol) in methanol (10 mL) was added K₂CO₃ (126 mg, 1.17 mmol) and the resultant mixture was stirred at room temperature for 30 minutes. The reaction mixture was poured onto H₂O, brought to pH=5 with 1 N aqueous HCl and extracted 3 times with CH₂Cl₂. The combined organic extract was dried over MgSO₄ and concentrated in vacuo to afford 3-(4-fluorophenyl)-N-(5-hydroxymethylpyrazin-2-yl)-2-(4-methanesulfonylphenyl)-propionamide (430 mg, 97%): ¹H NMR (400 MHz, CHLOROFORM-d) δ ppm 3.00-3.11 (s, m, 4H) 3.57 (dd, J=13.89, 8.34 Hz, 1H) 3.87 (t, J=7.58 Hz, 1H) 4.77 (s, 2 H) 6.92 (t, J=8.59 Hz, 2H) 7.08 (dd, J=8.59, 5.05 Hz, 2H) 7.55 (d, J=8.08 Hz, 2H) 7.71-8.04 (m, 3H) 8.21 (d, J=1.52 Hz, 1H) 9.44 (s, 1H).

The isomers were separated by chiral SCF chromatography to afford the front-running isomer (135.6 mg): and a back-running isomer (140 mg):

### Example 51

### 3-(4-Fluoro-phenyl)-2-(4-methanesulfonyl-phenyl)-N-oxazolo[4,5-c]pyridin-2-yl-propionamide

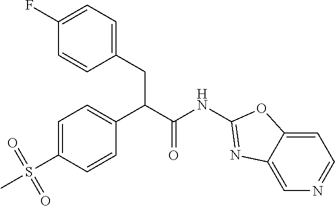

Preparation by a similar procedure to Example 40, except substituting 3-amino-4-hydroxypyridine for 2-amino-5-fluorophenol afforded 3-(4-fluoro-phenyl)-2-(4-methanesulfonyl-phenyl)-N-oxazolo[4,5-c]pyridin-2-yl-propionamide (54.8 mg, 12%): ¹H NMR (300 MHz, CHLOROFORM-d) δ ppm 2.94-3.13 (s, m, 4H) 3.57 (dd, J=13.85, 8.76 Hz, 1H) 6.89 (t, J=8.67 Hz, 2H) 7.12 (dd, J=8.76, 5.37 Hz, 2H) 7.39 (dd, J=5.46, 0.75 Hz, 1H) 7.57 (d, J=8.48 Hz, 2H) 7.83 (d, J=8.48 Hz, 2H) 8.44 (d, J=5.46 Hz, 1H) 8.79 (s, 1H).

### Example 52

### 3-(4-Fluoro-phenyl)-2-(4-methanesulfonyl-phenyl)-N-oxazolo[5,4-c]pyridin-2-yl-propionamide

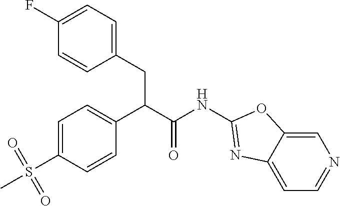

Preparation by a similar procedure to Example 40, except substituting 4-amino-3-hydroxypyridine for 2-amino-5-fluorophenol afforded 3-(4-fluorophenyl)-2-(4-methanesulfonyl-phenyl)-N-oxazolo[5,4-c]pyridin-2-yl-propionamide (51.2 mg, 11%): ¹H NMR (300 MHz, CHLOROFORM-d) δ ppm 2.95-3.12 (s, m, 5H) 3.57 (dd, J=13.56, 8.67 Hz, 1H) 6.89 (t, J=8.67 Hz, 2H) 7.12 (dd, J=8.67, 5.27 Hz, 2H) 7.39 (dd, J=5.46, 0.94 Hz, 1H) 7.57 (d, J=8.29 Hz, 2H) 7.83 (d, J=8.67 Hz, 2H) 8.43 (d, J=5.65 Hz, 1H) 8.78 (s, 1H).

### Example 53

### N-Benzooxazol-2-yl-2-(4-fluoro-phenyl)-3-(4-methanesulfonyl-phenyl)-propionamide

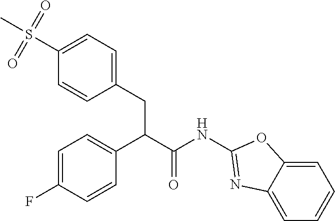

Step 1: To a solution of 4-fluorophenylacetic acid (1.0 g, 6.5 mmol) and 2-amino-benzoxazole (850 mg, 6.5 mmol) in CH₂Cl₂ (50 mL) was added DMAP (237 mg, 1.9 mmol) and EDC.HCl (1.5 g, 7.8 mmol). The reaction mixture was stirred at room temperature over night. The reaction mixture was concentrated in vacuo. Purification by chromatography (silica, 30% ethyl acetate/CH₂Cl₂) afforded N-benzooxazol-2-yl-2-(4-fluorophenyl)acetamide (1.44 g, 82%): ¹H NMR (300 MHz, DMSO-d₆) δ ppm 3.85 (s, 2H) 7.11-7.21 (m, 2H) 7.23-7.44 (m, 4H) 7.52-7.69 (m, 2H).

Step 2: To a slurry of sodium hydride (71 mg of 60% in oil dispersion, 1.78 mmol) in DMF (2 mL) under argon at 0° C. was added a solution of N-benzooxazol-2-yl-2-(4-fluorophenyl)acetamide (200 mg, 0.74 mmol) in DMF (6 mL). The reaction mixture was stirred at room temperature for 1 h. p-Methylsulfonylbenzyl chloride (159 mg, 0.77 mmol) in DMF (1 mL) was added. The reaction mixture was stirred at room temperature for 3 h and then quenched with saturated aqueous NH₄Cl. The resultant mixture was poured onto 0.1% aqueous HCl (25 mL) and extracted 3 times with CH₂Cl₂. The combined organic layer were dried over Na₂SO₄ and concentrated in vacuo. Residue was suspended in CH₂Cl₂ and filtered. The filtrate was concentrated in vacuo and purification by chromatography (silica, 50% ethyl acetate/hexanes) afforded N-benzooxazol-2-yl-2-(4-fluoro-phenyl)-3-(4-methanesulfonyl-phenyl)-propionamide (20.2 mg, 6%): ¹H NMR (300 MHz, CHLOROFORM-d) δ ppm 3.03 (s, 3H) 3.04-3.18 (m, 2H) 3.64 (dd, J=13.94, 8.29 Hz, 1H) 6.96 (t, J=8.67 Hz, 2H) 7.20-7.44 (m, 7H) 7.49 (d, J=7.16 Hz, 1H) 7.77 (d, J=8.29 Hz, 2H).

### Example 54

### N-Benzooxazol-2-yl-3-(4-fluoro-phenyl)-2-pyridin-4-yl-propionamide

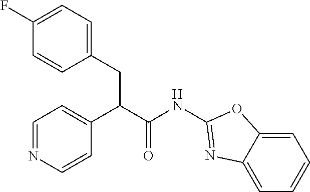

Step 1: To a solution of 4-pyridyl acetic acid hydrochloride (521 mg, 3 mmol) and 2-aminobenzoxazole (402 mg, 3 mmol) in CH₂Cl₂ (50 mL) was added DMAP (110 mg, 0.9 mmol) and EDC.HCl (690 mg, 4 mmol). The reaction mixture was stirred at room temperature over night. The reaction mixture was concentrated in vacuo. Purification by chromatography (silica, 2-5% methanol/CH₂Cl₂ gradient) afforded N-benzooxazol-2yl-2-pyridin-4-yl-acetamide (228 mg, 30%): ¹H NMR (300 MHz, METHANOL-d₄) δ ppm 5.05 (br. s., 2H) 7.19-7.35 (m, 2H) 7.39-7.49 (m, 3H) 7.54 (dt, J=7.25, 1.08 Hz, 1H) 8.39-8.56 (m, 2H).

Step 2: To a slurry of sodium hydride (86 mg of 60% in oil dispersion, 2.2 mmol) in DMF (3 mL) under argon at 0° C. was added a solution of N-benzooxazol-2yl-2-pyridin-4-yl-acetamide (228 mg, 0.9 mmol) in DMF (5 mL). The reaction mixture was stirred at room temperature for 1.25 h. 4-fluorobenzyl bromide (116 μL, 0.95 mmol) in DMF (1 mL) was added. The reaction mixture was stirred at room temperature for 3 h and then quenched with saturated aqueous NH₄Cl. The resultant mixture was poured onto 0.1% aqueous HCl (50 mL) and extracted 3 times with CH₂Cl₂. The combined organic layer was dried over MgSO₄ and concentrated in vacuo. Purification by chromatography (silica, 3% methanol in CH₂Cl₂) followed by trituration with ether afforded N-benzooxazol-2-yl-3-(4-fluoro-phenyl)-2-pyridin-4-yl-propionamide (95 mg, 29%): ¹H NMR (300 MHz, CHLOROFORM-d) δ ppm 2.99-3.11 (m, 1H) 3.50-3.61 (m, 1H) 6.89 (t, J=8.67 Hz, 2H) 7.10 (dd, J=8.67, 5.27 Hz, 2H) 7.23-7.35 (m, 5H) 7.40-7.46 (m, 1H) 8.50-8.56 (m, 2H).

### Example 55

### N-Benzooxazol-2-yl-2-(4-methanesulfonyl-phenyl)-3-pyridin-4-yl-propionamide

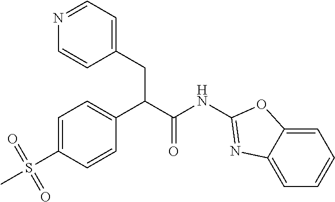

To a slurry of sodium hydride (29 mg of 60% in oil dispersion, 0.73 mmol) in DMF (1 mL) under nitrogen at 0° C. was added a solution of N-benzooxazol-2-yl-2-(4-methanesulfonylphenyl)acetamide (prepared as in example 5, 100 mg, 0.3 mmol) in DMF (2 mL). The reaction mixture was stirred at room temperature for 2 h. A suspension of 4-bromomethylpyridine hydrobromide (77 mg, 0.3 mmol) was suspended in DMF (1.5 mL) and to that was added sodium hydride (12 mg of 60% in oil dispersion, 0.3 mmol). The resulting 4-bromomethylpyridine solution was added to the above solution. The combined reaction mixture was stirred at room temperature for 2 h. The reaction mixture was quenched with saturated aqueous NH₄Cl, diluted with H₂O and extracted 1 time with ether and 2 times with CH₂Cl₂. The combined organic extracts were washed with saturated aqueous NaCl, dried over Na₂SO₄ and concentrated in vacuo. Purification by chromatography (silica, 1-3% methanol in CH₂Cl₂ gradient) afforded N-benzooxazol-2-yl-2-(4-methanesulfonyl-phenyl)-3-pyridin-4-yl-propionamide: ¹H NMR (300 MHz, CHLOROFORM-d) δ ppm 2.94-3.19 (s, m, 5H) 3.61 (dd, J=13.94, 8.67 Hz, 1H) 7.13 (d, J=5.65 Hz, 2H) 7.19-7.33 (m, 3H) 7.34-7.51 (m, 2H) 7.57 (d, J=8.29 Hz, 2H) 7.84 (d, J=8.29 Hz, 2H) 8.40 (d, J=4.90 Hz, 2H). Product was converted to the HCl salt with 1N HCl in ether to afford the salt as a white solid (51.6 mg, 37%).

### Biological Examples

### Intracellular Calcium Flux (FLIPR) Assay

### Compound and Reagent Preparation:

Stock solutions of compounds were prepared from powders as a 10 mM DMSO stock solution. These solutions were stored at RT during the two week period of these experiments to prevent freeze-thaw of the DMSO stocks. The DMSO stocks were added to the appropriate assay buffer at a concentration of 10 μM, and then diluted serially to the final concentrations that were tested. No observable precipitate was formed at any time during this process. The aqueous solutions of compounds as well as ATP (Sigma A7699) and BzATP (Sigma B6396) were prepared fresh for each day of experiment.

Cell Culture: 1321N1-hP2X₇ and HEK293-rP2X₇

1321N1 cells stably expressing the full length human P2X₇ gene (1321N1-hP2X₇) and HEK293 cells stably expressing the full length rat P2X₇ gene (HEK293-rP2X₇) were obtained from the Roche Cell Culture Facility. 1321N1-hP2X₇ cells were grown in Dulbecco's Modified Eagle's Medium (DMEM) high glucose supplemented with 10% FBS and 250 μg/mL G418. HEK293-rP2X₇ cells were grown in DMEM/F-12 supplemented with 10% FBS, 1 mM CaCl₂, 2 mM MgCl₂, 2 mM L-Glutamine and 500 μg/ml G418. Cells were split such that they never became >70% confluent.

### Intracellular Calcium Flux (FLIPR).

On the day prior to the experiment, 1321N1-hP2X₇ or HEK293-rP2X₇ cells were released into suspension with calcium-free PBS+Versene and washed by centrifugation with calcium-free PBS to remove the Versene. Cells were resuspended in growth medium at a density of 2.5×10⁵ cells/mL and seeded into black walled, clear bottom 96 well plates (50,000 cells/well) approximately 18 hr prior to intracellular calcium flux experiments.

On the day of the experiment, plates were washed with FLIPR buffer (calcium- and magnesium-free Hank's Balanced Salt Solution (HBSS) supplemented with 10 mM Hepes, 2.5 mM probenecid and 2 mM calcium chloride) using a BIO-TEK 96 channel plate washer and incubated with 2 mM fluo-3 dye at 37° C. for one hr. The dye was then removed by plate washing and the cells were allowed to equilibrate for 20 min at room temperature with antagonist or vehicle (FLIPR buffer). Agonist (100 μM BzATP final concentration for hP2X₇; 5 μM BzATP final concentration or rP2X₇) was added online with the FLIPR and fluorescence measurements made at 1 sec intervals for 60 sec followed by 3 sec intervals for a further 4 min (5 min total). A final addition of 5 μM ionomycin was made and the maximal BzATP-evoked fluorescence normalized to the maximal ionomycin-evoked fluorescence.

| Compound | FLIPR pIC50 hP2X7-1321n1 |
|---|---|
| I-1 | 6.41 |
| I-2 | 6.52 |
| I-3 | 5.93 |
| I-4 | 7.09 |
| I-5 | 6.27 |
| I-6 | 6.10 |
| I-7 | 7.20 |
| I-8 | 7.06 |
| I-9 | 6.64 |
| I-10 | 6.37 |
| I-11 | 7.09 |
| I-12 | 6.13 |
| I-13 | 6.09 |
| I-14 | 5.95 |
| I-15 | 6.65 |
| I-16 | 6.93 |
| I-17 | 5.97 |
| I-18 | 6.63 |
| I-19 | 6.15 |
| I-20 | 7.69 |
| I-21 | 6.19 |
| I-22 | 6.13 |
| I-23 | 6.19 |
| I-24 | 5.98 |
| I-25 | 6.07 |
| I-26 | 6.47 |
| I-27 | 6.69 |
| I-28 | 6.36 |
| I-29 | 6.30 |
| I-30 | 6.34 |
| I-31 | 6.55 |
| I-32 | 7.69 |
| I-33 | 6.46 |
| I-34 | 6.41 |
| I-35 | 6.33 |
| I-36 | 6.72 |
| I-37 | 6.78 |
| I-38 | 6.50 |
| I-39 | 6.90 |
| I-40 | 7.41 |
| I-41 | 6.94 |
| I-42 | 7.54 |
| I-43 | 6.99 |
| I-44 | 7.12 |
| I-45 | 6.21 |
| I-46 | 7.35 |
| I-47 | 6.77 |
| I-48 | 6.53 |
| I-49 | 6.06 |
| I-50a | 6.42 |
| I-50b | <5 |
| I-51 | 7.50 |
| I-52 | 7.18 |
| I-53 | 6.39 |
| I-54 | 7.51 |
| I-55 | 6.18 |

### Human Whole Blood IL-β Release Assay.

### Compound & Reagent Preparation:

10 mM stock solutions of compounds in DMSO (Sigma D2650) are prepared and used either fresh or after storage at −20° C. Appropriate (200×) serial dilutions of the compounds are made in DMSO, then freshly diluted 1 to 20 (10×) with Dulbecco's phosphate buffered saline (DPBS; Mediatech Inc., 21-030), such that final DMSO concentration in the blood always equals 0.5%.

30 mM ATP (Sigma A7699) is prepared immediately before use in 50 mM HEPES (Gibco 15630) and the pH is adjusted to 7.2 with 1M sodium hydroxide.

### Blood Donors:

Human blood donors are medication free and restricted from utilizing alcohol or caffeine for at least the 24 hr preceding collection. The blood is collected into sodium heparin vacutainer tubes and used the same day.

### Assay Method:

The OptEIA Human IL-β ELISA Set, OptEIA Coating Buffer, Assay Diluent and TMB Substrate Reagent Set used in the assay are commercially obtained from BD Pharmingen. Blood is diluted 1:1 with Dulbecco's PBS, LPS (Escherichia Coli 0127:B8, Sigma L3129) added to a final concentration of 25 ng/mL and incubated for 2 hr at 37° C. 48 μL of this LPS primed blood is added to 6 μL of the 10× compound in 5% DMSO/PBS in the appropriate well of a 96-well polypropylene plate. The blood and compound are mixed and allowed to incubate for 30 min at 37° C. 6 μl of 30 mM ATP is added to the LPS-primed blood+compound, mixed thoroughly and incubated for a further 30 min at 37° C. 96 μL of ELISA assay buffer is added to each well and the plate centrifuged at 4° C. 1,200 rpm for 10 min. Supernatant is removed and assayed for IL-β using the OptiEIA kit according to the manufacturer's protocol (Serum may be frozen at −\tilde{2}° C. prior to assay). IC₅₀s are calculated using XLfit.

### In Vivo Assay for Asthma and Lung Function.

BALb/cJ mice are immunized with a standard immunization protocol. Briefly, mice (N=8/group) are immunized i.p. with ovalbumin (OVA; 10 μg) in alum on days 0 and 14. Mice are then challenged with aerosolized OVA (5%) on day 21 and 22. Animals receive vehicle (p.o.) or a compound of the invention (100 mg/kg p.o.) all starting on day 20.

Lung function is evaluated on day 23 using the Buxco system to measure PenH in response to an aerosol methacholine challenge. Mice are then euthanized and plasma samples collected at the end of the study.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound of formula I: 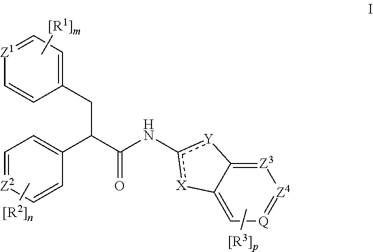
wherein:
Z¹ is CH, CR¹, or N;
Z² is CH, CR², or N;
Z³ is CH, CR³, or N;
Z⁴ is CH, CR³, or N;
Q is CH, CR³, or N;
wherein only one of Z³, Z⁴, and Q can be N;
X is N or O;
Y is N or O;
wherein both X and Y are not both N or O;
each — is a single or double bond, wherein both are not single or double;
each R¹ is independently halo, hydroxy, lower alkyl, lower alkoxy, lower hydroxyalkyl, —SO₂R, or lower haloalkyl;
m is 0, 1, 2, or 3;
each R² is independently halo, lower alkyl, lower hydroxyalkyl, lower alkoxy, C(═O)OR^{2′}, C(═O)N(R^{2′})₂, or S(═O)₂R^{2′};
R^{2′} is H, lower alkyl, lower alkoxy, amino, or lower hydroxyalkyl;
n is 0, 1, 2, or 3;
each R³ is halo, hydroxy, lower alkyl, lower alkoxy, lower hydroxyalkyl, lower haloalkyl, CO₂R³′, or S(═O)₂R^{3′};
each R^{3′} is independently H, lower alkyl, cycloalkyl, alkylamine,
or heterocycloalkyl;
p is 0, 1, 2, or 3;
or a pharmaceutically acceptable salt thereof.

2. The compound of claim 1, wherein X is O and Y is N.

3. The compound of claim 1, wherein X is N and Y is O.

4. The compound of claim 2, wherein Z³, Z⁴, and Q are CH or CR³.

5. The compound of claim 3, wherein Z³, Z⁴, and Q are CH or CR³.

6. The compound of claim 4, wherein m is 1.

7. The compound of claim 5, wherein m is 1.

8. The compound of claim 6, wherein R¹ is halo or lower alkyl.

9. The compound of claim 7, wherein R¹ is halo or lower alkyl.

10. The compound of claim 8, wherein R² is S(═O)₂R^{2′} and R^{2′} is lower alkyl.

11. The compound of claim 9, wherein R² is S(═O)₂R^{2′} and R^{2′} is lower alkyl

12. The compound of claim 10, wherein p is 0.

13. The compound of claim 11, wherein p is 0.

14. The compound of claim 12, wherein p is 1.

15. The compound of claim 13, wherein p is 1.

16. The compound of claim 1, wherein Z³ is N.

17. The compound of claim 1, wherein Z⁴ is N.

18. The compound of claim 1, wherein Q is N.

19. The compound of claim 1, wherein R² is C(═O)N(R^{2′})₂.

20. The compound of claim 1, wherein R² is C(═O)OR^{2′}.

21. A compound of Formula II 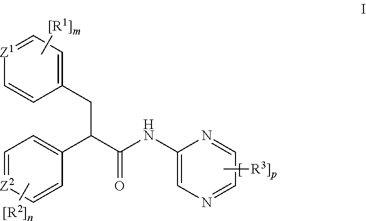
wherein:
Z¹ is CH, CR¹, or N;
Z² is CH, CR², or N;
each R¹ is independently halo, hydroxy, lower alkyl, lower alkoxy, lower hydroxyalkyl, or lower haloalkyl;
m is 0, 1, 2, or 3;
each R² is independently halo, lower alkyl, C(═O)R^{2′}, C(═)ON(R^{2′})₂, or S(═O)₂R^{2′};
each R^{2′} is independently H, lower alkyl, lower alkoxy, amino, or lower hydroxyalkyl;
n is 0, 1, 2, or 3;
each R³ is independently halo, hydroxy, lower alkyl, lower alkoxy, lower hydroxyalkyl, alkoxyalkyl, lower haloalkyl, alkylamino, thioalkyl, CH₂(O)C(═O)R^{3′}, C(═O)OR^{3′}, CH₂(O)R^{3′}, or CH₂N(R^{3′})₂;
each R3′ is independently H, lower alkyl, lower alkoxy, or phenyl lower alkyl,
p is 0, 1, 2, or 3;
or a pharmaceutically acceptable salt thereof.

22. The compound of claim 21, wherein m is 1 and n is 1.

23. The compound of claim 22, wherein R² is S(═O)₂CH₃.

24. The compound of claim 23, wherein p is 1.

25. The compound of claim 24, wherein R³ is halo.

26. The compound of claim 24, wherein R³ is lower hydroxyalkyl.

27. The compound of claim 24, wherein R³ is lower alkyl.

28. A compound selected from the group consisting of:
N-(5-Bromo-pyrazin-2-yl)-2-(4-methanesulfonyl-phenyl)-3-o-tolyl-propionamide;
N-Benzooxazol-2-yl-2-(4-methanesulfonyl-phenyl)-3-o-tolyl-propionamide;
N-(5-Chloro-benzooxazol-2-yl)-2-(4-methanesulfonyl-phenyl)-3-o-tolyl-propionamide;
N-Benzooxazol-2-yl-2-(4-methanesulfonyl-phenyl)-3-phenyl-propionamide;
N-Benzooxazol-2-yl-3-(2-chloro-phenyl)-2-(4-methanesulfonyl-phenyl)-propionamide;
N-Benzooxazol-2-yl-3-(2-chloro-4-fluoro-phenyl)-2-(4-methanesulfonyl-phenyl)-propionamide;
N-Benzooxazol-2-yl-3-(4-fluoro-phenyl)-2-(4-methanesulfonyl-phenyl)-propionamide;
N-Benzooxazol-2-yl-3-(2-fluoro-phenyl)-2-(4-methanesulfonyl-phenyl)-propionamide;
N-Benzooxazol-2-yl-2-(4-methanesulfonyl-phenyl)-3-(4-methoxy-phenyl)-propionamide;
(S)—N-Benzooxazol-2-yl-2-(4-methanesulfonyl-phenyl)-3-o-tolyl-propionamide;
(R)—N-Benzooxazol-2-yl-2-(4-methanesulfonyl-phenyl)-3-o-tolyl-propionamide;
N-Benzooxazol-2-yl-3-(4-fluoro-phenyl)-2-(3-methanesulfonyl-phenyl)-propionamide;
N-(5-Bromo-pyrazin-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-methylsulfanyl-pyrazin-2-yl)-propionamide;
5-[3-(4-Fluoro-phenyl)-2-(4-methanesulfonyl-phenyl)-propionylamino]-pyrazine-2-carboxylic acid ethyl ester;
3-(4-Fluoro-phenyl)-N-(5-hydroxymethyl-pyrazin-2-yl)-2-(4-methanesulfonyl-phenyl)-propionamide;
3-(4-Fluoro-phenyl)-N-[5-(1-hydroxy-1-methyl-ethyl)-pyrazin-2-yl]-2-(4-methanesulfonyl-phenyl)-propionamide;
3-(4-Fluoro-phenyl)-2-(4-methanesulfonyl-phenyl)-N-oxazolo[4,5-b]pyridin-2-yl-propionamide;
3-(4-Fluoro-phenyl)-2-(4-methanesulfonyl-phenyl)-N-(5-methyl-pyrazin-2-yl)-propionamide;
3-(4-Fluoro-phenyl)-N-(6-hydroxymethyl-benzooxazol-2-yl)-2-(4-methanesulfonyl-phenyl)-propionamide;
3-(4-Fluoro-phenyl)-2-(4-methanesulfonyl-phenyl)-N-(5-methoxymethyl-pyrazin-2-yl)-propionamide;
N-(5-Ethoxymethyl-pyrazin-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-{[(2-methoxy-ethyl)-methyl-amino]-methyl}-pyrazin-2-yl)-propionamide;
N-{5-[(Benzyl-methyl-amino)-methyl]-pyrazin-2-yl}-3-(4-fluoro-phenyl)-2-(4-methanesulfonyl-phenyl)-propionamide;
N-(6-Chloro-pyrazin-2-yl)-3-(4-fluoro-phenyl)-2-(4-methanesulfonyl-phenyl)-propionamide;
3-(3-Fluoro-phenyl)-N-(5-hydroxymethyl-pyrazin-2-yl)-2-(4-methanesulfonyl-phenyl)-propionamide;
N-Benzooxazol-2-yl-3-(4-fluoro-phenyl)-2-(4-methanesulfonyl-2-methyl-phenyl)-propionamide;
N-Benzooxazol-2-yl-2,3-bis-(4-fluoro-phenyl)-propionamide;
N-Benzooxazol-2-yl-2-(2,4-difluoro-phenyl)-3-(4-fluoro-phenyl)-propionamide;
4-[1-(Benzooxazol-2-ylcarbamoyl)-2-(4-fluoro-phenyl)-ethyl]-benzoic acid ethyl ester;
N-Benzooxazol-2-yl-3-(4-fluoro-phenyl)-2-[4-(1-hydroxy-1-methyl-ethyl)-phenyl]-propionamide;
4-[1-(Benzooxazol-2-ylcarbamoyl)-2-(4-fluoro-phenyl)-ethyl]-benzamide;
4-[1-(Benzooxazol-2-ylcarbamoyl)-2-(4-fluoro-phenyl)-ethyl]-N-(2-hydroxy-ethyl)-benzamide;
4-[1-(Benzooxazol-2-ylcarbamoyl)-2-(4-fluoro-phenyl)-ethyl]-N-(2-dimethylamino-ethyl)-benzamide;
3-(4-Fluoro-phenyl)-2-(4-methanesulfonyl-phenyl)-N-(4-methyl-benzooxazol-2-yl)-propionamide;
3-(4-Fluoro-phenyl)-2-(4-methanesulfonyl-phenyl)-N-(6-methyl-benzooxazol-2-yl)-propionamide;
3-(4-Fluoro-phenyl)-2-(4-methanesulfonyl-phenyl)-N-(5-methyl-benzooxazol-2-yl)-propionamide;
N-(6-Chloro-benzooxazol-2-yl)-3-(4-fluoro-phenyl)-2-(4-methanesulfonyl-phenyl)-propionamide;
N-(5-Chloro-benzooxazol-2-yl)-3-(4-fluoro-phenyl)-2-(4-methanesulfonyl-phenyl)-propionamide;
N-(6-Fluoro-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-trifluoromethyl-benzooxazol-2-yl)-propionamide;
3-(4-Fluoro-phenyl)-2-(4-methanesulfonyl-phenyl)-N-(7-methyl-benzooxazol-2-yl)-propionamide;
N-(5,7-Dimethyl-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-(5-methoxy-benzooxazol-2-yl)-propionamide;
2-[3-(4-Fluoro-phenyl)-2-(4-methanesulfonyl-phenyl)-propionylamino]-benzooxazole-6-carboxylic acid methyl ester;
N-(5-Fluoro-benzooxazol-2-yl)-3-(4-fluoro-phenyl)-2-(4-methanesulfonyl-phenyl)-propionamide;
3-(4-Fluoro-phenyl)-2-(4-methanesulfonyl-phenyl)-N-[5-(pyrrolidine-1-sulfonyl)-benzooxazol-2-yl]-propionamide;
3-(4-Fluoro-phenyl)-2-(4-methanesulfonyl-phenyl)-N-(5-phenyl-oxazol-2-yl)-propionamide;
Acetic acid 5-[3-(4-fluoro-phenyl)-2-(4-methanesulfonyl-phenyl)-propionylamino]-pyrazin-2-ylmethyl ester;
(R)-3-(4-Fluoro-phenyl)-N-(5-hydroxymethyl-pyrazin-2-yl)-2-(4-methanesulfonyl-phenyl)-propionamide;
(S)-3-(4-Fluoro-phenyl)-N-(5-hydroxymethyl-pyrazin-2-yl)-2-(4-methanesulfonyl-phenyl)-propionamide;
3-(4-Fluoro-phenyl)-2-(4-methanesulfonyl-phenyl)-N-oxazolo[4,5-c]pyridin-2-yl-propionamide;
3-(4-Fluoro-phenyl)-2-(4-methanesulfonyl-phenyl)-N-oxazolo[5,4-c]pyridin-2-yl-propionamide;
N-Benzooxazol-2-yl-2-(4-fluoro-phenyl)-3-(4-methanesulfonyl-phenyl)-propionamide;
N-Benzooxazol-2-yl-3-(4-fluoro-phenyl)-2-pyridin-4-yl-propionamide; and
N-Benzooxazol-2-yl-2-(4-methanesulfonyl-phenyl)-3-pyridin-4-yl-propionamide.

29. A pharmaceutical composition comprising:
(a) a pharmaceutically acceptable carrier; and
(b) a compound of claim 28.

30. A method for treating arthritis, said method comprising administering to a subject in need thereof a therapeutically effective amount of a compound of claim 28.

31. A method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof a therapeutically effective amount of a compound of claim 28.

32. A method for treating a respiratory disorder selected from chronic obstructive pulmonary disorder (COPD), asthma, and bronchospasm, said method comprising administering to a subject in need thereof a therapeutically effective amount of a compound of claim 28.

33. A method for treating diabetes, said method comprising administering to a subject in need thereof a therapeutically effective amount of a compound of claim 28.
